SEQUENCE LISTING (52 sequences)

SEQ ID NO: 1 - DNA, 1689 bp, Corynebacterium jeikeium
atgactaactcttctgcaaccagcaatccccaacccagcgacgtcgaaatcgcacaggcccacaccctggagcccatcaccacgatcgcggagcgcgccggcatcccggaggccgctttgattccctacggccgtacaaaggccaagatcgacgttcccgctctgcgggcggagcgtgagggcgtcaataaaaagggcaagctcgtgctcgtgaccgcaatgagcccgacccccgcgggtgaaggcaagtccacggtgctgatcggcttggcggacgcggtgcgcacggcagggcgccagacgatggtggcgatccgcgagccgtcccaggggccagtcatgggcatcaagggcggcgctgctggcggcggttatgcgcagatcgtgccgatggaagacatcaacctgcacttcacaggcgacatgcacgccatcaccgccgcgacgaacactctggcggcgatggtggacaaccacgtgcagcacggcaatgctctgggcatcgacccgcggcgggtgacgtggcgacgctgccttgacgtcaacgaccgctcgctgcgccatgtagtcacaggcctaggcggtcctggtcagggcacgccccgcgagggcggattcgatatcacggcggccagcgagatcatggctatcctctgtctggccaccgacctggaggatctaaagaagcgcatcgggcgcatcgtggtgggccagacctacgaccgtcgaccagtcaccgcgggcgacctgaagtgcgcgggcgctatcaccgccctgctgcgcgacgcgatcaacccgaacctggtgcagaccctgggcggcactcccgccctggtgcacggcgggcctttcgccaacatcgcgcacggctgtaactctctgatcgcaaccaccacggcgctggatctatcagaagttgtgctgaccgaggctggtttcggcagcgacctgggtgcggagaagttcttcgacatcaagtcccgcgctggcgacctcgacgtcgccgcaacggttgtggtggccaccatccgttcgctgaagcacaacggcgattccgtgttgaaggccggtctggccaacctggagcgccacgtcagtaacatccgcaagttcggcgtggaacccgtggtggcgctgaacctgtttagctccgataccgccgcggagcgctctatggtcgccgattggggcgagcagttcggtgtgcgcgtggtggagtgcagcgtgtgggctgagggcggtgccggcgcggcggatctggccactgcggtgctggaggtcgttgatggtgtttctgatgaggacgcctccagctccagccaccagatctatcagcccgtcgatggcgtggaagccaccctgcacacacttgccacggaaatctacggcgcggcggatgtgcagttcggcccgcaggctctgaaggacctggcgtttctgaaggacaacggctgggacaagctgccagtgtgcgtgtcgaagacacagtactccttcagcgatgaccccagcgcgctgggcgcgccgagcggccacaccctgcatgtccgcgagctggtgccacgcatcggcgccggattcgtggtggctctcaccggcgacgtgatgactctgccgggtctgccgaagaaacccgcagccgagcgtattgatgtgaacgcgcagggggttatctcggggctgttctaa

SEQ ID NO: 2 - protein, 562 aa, Corynebacterium jeikeium
MTNSSATSNPQPSDVEIAQAHTLEPITTIAERAGIPEAALIPYGRTKAKIDVPALRAEREGVNKKGKLVLVTAMSPTPAGEGKSTVLIGLADAVRTAGRQTMVAIREPSQGPVMGIKGGAAGGGYAQIVPMEDINLHFTGDMHAITAATNTLAAMVDNHVQHGNALGIDPRRVTWRRCLDVNDRSLRHVVTGLGGPGQGTPREGGFDITAASEIMAILCLATDLEDLKKRIGRIVVGQTYDRRPVTAGDLKCAGAITALLRDAINPNLVQTLGGTPALVHGGPFANIAHGCNSLIATTTALDLSEVVLTEAGFGSDLGAEKFFDIKSRAGDLDVAATVVVATIRSLKHNGDSVLKAGLANLERHVSNIRKFGVEPVVALNLFSSDTAAERSMVADWGEQFGVRVVECSVWAEGGAGAADLATAVLEVVDGVSDEDASSSSHQIYQPVDGVEATLHTLATEIYGAADVQFGPQALKDLAFLKDNGWDKLPVCVSKTQYSFSDDPSALGAPSGHTLHVRELVPRIGAGFVVALTGDVMTLPGLPKKPAAERIDVNAQGVISGLF

SEQ ID NO: 3 - DNA, 192 bp, artificial
  promotor P3119 = PSOD
gagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttaccc

SEQ ID NO: 4 - DNA, 184 bp, artificial
  promotor P497 = PgroES
ggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatc

SEQ ID NO: 5 - DNA, 199 bp, artificial
  promotor P1284 = PEFTU
ggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacataca

SEQ ID NO: 6 - DNA, 114 bp, artificial
  promotor ... = lpR
gtcgactcatacgttaaatctatcaccgcaagggataaatatctaacaccgtgcgtgttgactattttacctctggcggtgataatggttgcatgtactaaggaggattaatta

SEQ ID NO: 7 - DNA, 915 bp, Corynebacterium glutamicum
atgaccccgagttctcctgaagttcgtaatcgtccgagcgctgcgcctgaagagcgtcagtttgtgctcacttttggctgccctgactccactggaattgtggcgaagttgtcgtcgttcctagctgagcgtgggggttggattactgaggctggatatttcacggatcctgattcgaattggttctttactcgtcaggcgattcgcgctgagtcgattgataccacgattgagcagttgcgggaggagttcgctccgcttgcggaggagttcggcccgagggctaagtggagtttcactgacactgcgcaggtgaagaaggctgtgttgttggtgtctaaggagggccactgcttgcacgatttgttaggtcgtgtggctgagaatgattatccgatggaagttgttgcggttgtgggtaaccatgagaacttgcgttatattgcggagaaccataatgttccgtttttccatgtgccgtttcctaaggatgcggttggtaagcggaaggcgtttgaccaggtcgctgagattgtgaatggttatgatccggatgcgattgttttggctcgttttatgcagattttgccgccggatttgtgtgagatgtgggctggtcgtgtgttgaatattcatcacagtttcttgccgtcgtttatgggtgcgcgcccgtatcatcaggcgtatagccgtggtgtgaagttgattggtgcgacctgccattatgcgactggggatctggatgatggtccgatcattgagcaggatgttattcgtgtgacgcataaggatacgccgactgagatgcagcgtttgggccgcgatgcggagaagcaggtgctggctcgcggtttgcgtttccacttggaggaccgggtgctggtttacggtaaccgcacggttgtctttgattaa

SEQ ID NO: 8 - protein, 304 aa, Corynebacterium glutamicum
MTPSSPEVRNRPSAAPEERQFVLTFGCPDSTGIVAKLSSFLAERGGWITEAGYFTDPDSNWFFTRQAIRAESIDTTIEQLREEFAPLAEEFGPRAKWSFTDTAQVKKAVLLVSKEGHCLHDLLGRVAENDYPMEVVAVVGNHENLRYIAENHNVPFFHVPFPKDAVGKRKAFDQVAEIVNGYDPDAIVLARFMQILPPDLCEMWAGRVLNIHHSFLPSFMGARPYHQAYSRGVKLIGATCHYATGDLDDGPIIEQDVIRVTHKDTPTEMQRLGRDAEKQVLARGLRFHLEDRVLVYGNRTVVFD

SEQ ID NO: 9 - DNA, 2952 bp, Corynebacterium jeikeium
atgtcttctgcagctactcgccgtaattcagcccccttcgttcagcgccacatcggcccaaaccaggccgatacccaggagatcctcgattacctgggctatgaatcttccgccgcgctggccgacgatgccctcccgaagtcgatccgccaggcaggcccgatcggcctgccggaggcactggatgagacggacaccctggccgccctgcgtgcttacgctgacaagaacgtgcagaagcagcagctgatcggcaacggttacttcgacacgatcactccggccgtgattcgccgcaacgtggtggagaacccgggctggtacaccgcctacaccccctaccagccggaaatctcccaggggcgcctcgaggccctgctgaacttccagacgatggtgcaggacctgaccggcctgccagtggccggtgcttcgctgttagacgaagccaccgcagtggccgaggccgtgcagctgatggctcgcggcaatgcgaaggctgccaagaagggcggcgtggtgctgctggattcctccctgcaccagcagtccatcaccgtaaccctggcgcgcgctgaggctgcgggtatcccggtggaggtcgtggacctggacggcgaagatgctaccgccgcgtttgagggccgcgagaacctcgtcggcgtggtgctttccaaccccggctccaccggccgcgtccgcgacctgtccggtctgatctctgcggcgaaggagaccggcgctctggtgacggtcgcttgtgacctgctggctcaggttctggtgacctccccgggctcccaaggtgccgacattgctgtcggctccgcccagcgcttcggcgtgccgctattcttcggtggcccgcacgcgggcttcatctcctgcaccgaggctctgcagcgtaagctgccgggccgcatcgtgggcgtgtccgtggatgccgagggcaccccggcctaccgcttggctctgcagacccgtgagcagcacattcgccgcgacaaggccaccagtaacatctgtaccgctcaggctctgctggccgtcgttgccggtttctacgcggtctggcacggcccagccggcctgcgcgccatcgccgagggggtgcacgcccgcgcgaccgccctggccgttgccctgtccgaggctggcctgacgctggcgcacgataccttcttcgacaccgtcaccgttgacgtgtctggttcttctcttggtgacgcccccacggctctgcgcgccgcagccgaggcaggctacaacctgcgccaggttaacgattccttcgtcggcatctctgtcggcgagtccaccacggacgaggacattgccaagctgatcgaggtgctgggctcccgcaccggcgaggtcaactccgcgagcttcgacgtcaccgccggcccgctgggcgaggccggcgtgctgcgcgcggaggacgaggagattctgacccacccgatcttcaccgccatcacttccgagacccagatgatgcgctacatgcgcaagctggccgaccgcgacctggcgctggatcgtacgatgatcccgctgggctcctgcaccatgaagctgaacgcggccgtctccatggagccgatcacctggcctggcttcgcaggcatccacccgcacgtcccggccgagcaggcgcagggctggctggagctcatcgaggacctggaggagcgcctggcgaagatcaccggctacgccaaggtttccgtccagccgaacgcgggctcccagggcgagttcgccggcctgctggcgatccaccgctaccaccagtcccgcggcgacgatcagcgtgacatcgttctgatcccggcctccgcgcacggcaccaacgctgcctctgcagcgctggcgggcctgaaggtcgtggctgtgaagaatgccgaagacggctccatcgacgtgccggacctggaggccaagctggagaagtacggcgagcagaccgccgccatcatgctgacctacccctccacccacggcgtgttcgaggagcaggtgcgcgacgtctgccagaaggttcacgacgctggcggccaggtgtacgtcgacggcgctaacctgaacgccctggtcggcctggcccagccgggcgagttcggcggcgacgtatcccacctgaacctgcacaagaccttcaccatcccgcacggtggtggcggcccgggcgttggcccggtgtgcgtggcagagcacctgatcccgttcctgcccaccgacccgaacgccgacgttatcgagggcgatgctgccctgcagtccggccagccggtctccggcgcgcagtacggctccgctggcgtgctgccgatcacctggtcctacatcgcacagatgggcgacgaaggcctgactgaggcctcccgcatggccctggtgaacgccaactacgtttcccgcaagctggaggactactacccgacgctgtacaagggcgacaccggcctggtcgcccacgagtgcatcctggacctgcgtgaactcacgaaggcctccggcatcaccgccgaggacgtatccaagcgcctgatggacttcggcttccacgccccgaccttggccttcccggtcgccggcaccctgatgatggagcccaccgagtcggaggacaaggaagagctggaccgcttcatcgaagcgatgatcaccatccacggggagatccaggaagttatcgacggcaaggtcaccgccgaacagtccgttctgcgccacgcgccgtttaccgcctattctgttgtgcgggacgacttcgaggaagctgtatccggtggtcacttcagccgtgccaaggccgcatacccggtggctagcctgcgacacaccaagtacttcacccccgtgcgtcgtatcgacaacgcctatggcgaccgcaatctcgtgtgcacctgcccgcccctggaagacttcgcaattaacgaggactag

SEQ ID NO: 10 - protein, 983 aa, Corynebacterium jeikeium
MSSAATRRNSAPFVQRHIGPNQADTQEILDYLGYESSAALADDALPKSIRQAGPIGLPEALDETDTLAALRAYADKNVQKQQLIGNGYFDTITPAVIRRNVVENPGWYTAYTPYQPEISQGRLEALLNFQTMVQDLTGLPVAGASLLDEATAVAEAVQLMARGNAKAAKKGGVVLLDSSLHQQSITVTLARAEAAGIPVEVVDLDGEDATAAFEGRENLVGVVLSNPGSTGRVRDLSGLISAAKETGALVTVACDLLAQVLVTSPGSQGADIAVGSAQRFGVPLFFGGPHAGFISCTEALQRKLPGRIVGVSVDAEGTPAYRLALQTREQHIRRDKATSNICTAQALLAVVAGFYAVWHGPAGLRAIAEGVHARATALAVALSEAGLTLAHDTFFDTVTVDVSGSSLGDAPTALRAAAEAGYNLRQVNDSFVGISVGESTTDEDIAKLIEVLGSRTGEVNSASFDVTAGPLGEAGVLRAEDEEILTHPIFTAITSETQMMRYMRKLADRDLALDRTMIPLGSCTMKLNAAVSMEPITWPGFAGIHPHVPAEQAQGWLELIEDLEERLAKITGYAKVSVQPNAGSQGEFAGLLAIHRYHQSRGDDQRDIVLIPASAHGTNAASAALAGLKVVAVKNAEDGSIDVPDLEAKLEKYGEQTAAIMLTYPSTHGVFEEQVRDVCQKVHDAGGQVYVDGANLNALVGLAQPGEFGGDVSHLNLHKTFTIPHGGGGPGVGPVCVAEHLIPFLPTDPNADVIEGDAALQSGQPVSGAQYGSAGVLPITWSYIAQMGDEGLTEASRMALVNANYVSRKLEDYYPTLYKGDTGLVAHECILDLRELTKASGITAEDVSKRLMDFGFHAPTLAFPVAGTLMMEPTESEDKEELDRFIEAMITIHGEIQEVIDGKVTAEQSVLRHAPFTAYSVVRDDFEEAVSGGHFSRAKAAYPVASLRHTKYFTPVRRIDNAYGDRNLVCTCPPLEDFAINED

SEQ ID NO: 11 - DNA, 384 bp, Corynebacterium jeikeium
atgactgcactgccaactgacttcctgtactccgaagagcacgagtgggttaacacctccgctgttgttgagggcgagaccgtgcgcgtgggcattacccacatcgccgctgaggcgctgggtgacatcgtgttcgtcgagctgccggaggttggctccgaggttgaggccggcgaggctttcggcgaggttgagtccaccaagtccgtttccgacatctacgcaccggtttctggcgaggttgtggctgtcaacgaggcgctggaagacaacgctggcctgatcaacgaagatccatacggtgagggctggctgtacgaggtcaaggtgaccgaggccggcgagctgatggaggctgaggcttaccaggcggctaacgagtaa

SEQ ID NO: 12 - protein, 127 aa, Corynebacterium jeikeium
MTALPTDFLYSEEHEWVNTSAVVEGETVRVGITHIAAEALGDIVFVELPEVGSEVEAGEAFGEVESTKSVSDIYAPVSGEVVAVNEALEDNAGLINEDPYGEGWLYEVKVTEAGELMEAEAYQAANE

SEQ ID NO: 13 - DNA, 1170 bp, Corynebacterium jeikeium
atgaccgaacttaagaagaccgcgctgcacctggtgcacgagaagttgggcgcgcgatttaccgacttcggcggctgggacatgcctctgaagtacagcagtgagctggacgagcaccacgctgtacgcaatgccgtgggcgtattcgacctctcccacatgggtgaggttcgcgtgaccggcccgcaggcagcggagttcctggaccacgcgctgatttcgaagctgtcggcagtgaaggtcggcaaggcgaagtactcgatgatctgcaccgaatccggtggcatcatcgacgacctgatcacctaccgcctgggcgacaacgagttcctgatcgtgccgaacgcgggcaacgtggacaacgtggtctccgcactgcagggccgcaccgagggctttgacgtggaggttaacaacgagtccgatgcgacctccatgatcgccgtacaggggcccaaggccgcgcaggcgatgctggagatcgtggagaacgtcgtggatgcacccgaggcatccggcgcgggcgagaccgttgccgaggctatcgaggggctgggttactacgcggcattcagcggtgttgccgcaggtcagcccgtgctggtggcccgcacaggctataccggcgaggacggtttcgagctgatcgtggctaacgatggtgcggagaccgtgtggaccaaggctatggaccaggctgcgcagctgggtggcctgccgtgtggcctggcctgccgcgacaccctgcgcctggaggctggcatgccgctgtacggcaacgagctatcgctgaagctcaccccggtcgatgctgggctgggcattcttgcggcgacgaagtctaaggactctttcgttggtcgtgacgccatcgtttccgccaaggaaaagggtacccagcaggtacttatcgggctggcgggcgagggtcgccgcgctgcccgtgggggatacgaggtgtttgccggtgacggcgagaaggccatcggtgccgtgacctccggtgcactgtcgccgacgctgggccacccggtggcattggcatacgtcgcgaagtccgcagtgtcctccggcgcggccgctgagggtgcgaccgtggaggtagacatccgcggcaagcgctttgaatacaaggttgtggcgctgccgttctactcccgcgagaagtaa

SEQ ID NO: 14 - protein, 389 aa, Corynebacterium jeikeium
MTELKKTALHLVHEKLGARFTDFGGWDMPLKYSSELDEHHAVRNAVGVFDLSHMGEVRVTGPQAAEFLDHALISKLSAVKVGKAKYSMICTESGGIIDDLITYRLGDNEFLIVPNAGNVDNVVSALQGRTEGFDVEVNNESDATSMIAVQGPKAAQAMLEIVENVVDAPEASGAGETVAEAIEGLGYYAAFSGVAAGQPVLVARTGYTGEDGFELIVANDGAETVWTKAMDQAAQLGGLPCGLACRDTLRLEAGMPLYGNELSLKLTPVDAGLGILAATKSKDSFVGRDAIVSAKEKGTQQVLIGLAGEGRRAARGGYEVFAGDGEKAIGAVTSGALSPTLGHPVALAYVAKSAVSSGAAAEGATVEVDIRGKRFEYKVVALPFYSREK

SEQ ID NO: 15 - DNA, 1017 bp, Escherichia coli
atgtccacattacgcctgctcatctctgactcttacgacccgtggtttaacctggcggtggaagagtgtatttttcgccaaatgcccgccacgcagcgcgttctgtttctctggcgcaatgccgacacggtagtaattggtcgcgcgcagaacccgtggaaagagtgtaatacccggcggatggaagaagataacgtccgcctggcgcggcgcagtagcggtggcggcgcggtgttccacgatctcggcaatacctgctttacctttatggctggcaagccggagtacgataaaactatctccacgtcgattgtgctcaatgcgctgaacgcgctcggcgtcagcgccgaagcgtccggacgtaacgatctggtggtgaaaaccgtcgaaggcgaccgcaaagtctcaggctcggcctatcgcgaaaccaaagatcgcggcttccaccacggcaccttgctactcaatgccgacctcagccgcctggcaaactatctcaatccggataaaaagaaactggcggcgaaaggcattacgtcggtacgttcccgcgtgaccaacctcaccgagctgttgccggggatcacccatgagcaggtttgcgaggccataaccgaggcctttttcgcccattatggcgagcgcgtggaagcggaaatcatctccccgaacaaaacgccagacttgccaaacttcgccgaaacctttgcccgccagagtagctgggaatggaacttcggtcaggctccggcattctcgcatctgctggatgaacgctttacctggggcggcgtggaactgcatttcgacgttgaaaaaggccatatcacccgcgcccaggtgtttaccgacagcctcaaccccgcgccgctggaagccctcgccggacgactgcaaggctgcctgtaccgcgcagatatgctgcaacaggagtgcgaagcgctgttggttgacttcccggaacaggaaaaagagctacgggagttatcggcatggatggcgggggctgtaaggtag

SEQ ID NO: 16 - protein, 338 aa, Escherichia coli
MSTLRLLISDSYDPWFNLAVEECIFRQMPATQRVLFLWRNADTVVIGRAQNPWKECNTRRMEEDNVRLARRSSGGGAVFHDLGNTCFTFMAGKPEYDKTISTSIVLNALNALGVSAEASGRNDLVVKTVEGDRKVSGSAYRETKDRGFHHGTLLLNADLSRLANYLNPDKKKLAAKGITSVRSRVTNLTELLPGITHEQVCEAITEAFFAHYGERVEAEIISPNKTPDLPNFAETFARQSSWEWNFGQAPAFSHLLDERFTWGGVELHFDVEKGHITRAQVFTDSLNPAPLEALAGRLQGCLYRADMLQQECEALLVDFPEQEKELRELSAWMAGAVR

SEQ ID NO: 17 - DNA, 1050 bp, Corynebacterium jeikeium
atgagtgtgaccgcagatggacgccggatgctacgcatcgaggcgaagaatgccgaaacccccatcgagtcgaagcctcggtggatccgcacgaccgcgaaggtcggcccggagtatcgggatattaagaatcgcgtgaagggcgctggcctgcacaccgtgtgccaagaggctggctgcccgaatatcaacgagtgctgggaggaccgcgaggcgacgttcctcatcggcggtgatacgtgttcccgccgttgcgacttctgccagattaagtccggccgcccgtccccgctggatatggacgagccacgccgcgtggcggagaatgtccgcgagatgggtctgcgctacgccaccatcaccggcgtgacgcgcgatgacctggatgatgagggcgcgtggctgtatgccgaggttgtgaagaagattcacgagctgaacccgaacacgggtgtggagaatctgacgccagatttttccaaccgcccggagctgctgaaggtcgtcttcgattcccagccggaggtgtttgcccacaacctggagacagttccgcgcatctttaagcgcatccgcccggcctttaagtacgaccgttccctggaggtcattcaggcagctcacgattacggcctggtgacgaagtccaacctgatcctgggcatgggtgagaagaaggaagaggtccgcgcggctatcaaggacctggcagacgccggcaccgacattctgacgattacccagtacctgcgcccgtcttccatgcaccacccgattgagcgttgggtgaagccggaggagttcatggagcactccgacgcagcctacgagctgggcatcaaggctgttatgtccggcccgttggtgcgttcctcttaccgcgccggccgcctgtacgcgcaggccaagcaggcgcgcggcgaggctattccggagaacctgaagcacttggaggagactctcgattccaccacgtcgcaggaggcctctacactgctggagcgctacggcgcttcggaggacacgccggtcactgcgtcgcgccgctag

SEQ ID NO: 18 - protein, 349 aa, Corynebacterium jeikeium
MSVTADGRRMLRIEAKNAETPIESKPRWIRTTAKVGPEYRDIKNRVKGAGLHTVCQEAGCPNINECWEDREATFLIGGDTCSRRCDFCQIKSGRPSPLDMDEPRRVAENVREMGLRYATITGVTRDDLDDEGAWLYAEVVKKIHELNPNTGVENLTPDFSNRPELLKVVFDSQPEVFAHNLETVPRIFKRIRPAFKYDRSLEVIQAAHDYGLVTKSNLILGMGEKKEEVRAAIKDLADAGTDILTITQYLRPSSMHHPIERWVKPEEFMEHSDAAYELGIKAVMSGPLVRSSYRAGRLYAQAKQARGEAIPENLKHLEETLDSTTSQEASTLLERYGASEDTPVTASRR

SEQ ID NO: 19 - DNA, 759 bp, Corynebacterium jeikeium
atgggattccagcaaggcagcatccgcaaagcaaacatgggcacaaccggcactaacgacggggcaaccaccccaccagccaacaccagcaccccagccgtagacatcgacgtccgcgacctgggcaccgtggactacgaggacacttggcacctccaggcaaatctcgccgcccagcgcgccgaagaaaaaatcccagacaccatcctcctactccagcatccgccgacgtacaccgccggcaagcgcacccaggattccgaccgccctaccaacggcctgccagtagtcgacgtcgaccgcggaggccgcatcacctggcatggacccggccagttggtcgcatatcccatcatcaagctggccgacccggtggacgtggtcgattatgtccgccgcctggagcaggcgctaatccagacttgtgaagatcttggcctgcacggcaccggccgcgtagaggggcgttcgggcgtgtggctgcctgcgggcgtcattaatggcgagctcaagcccgcacgtaagatagccgcgatcggcatccgcgtgacgcgcggcgtgaccatgcacggagtggccctcaactgcgataacaccatggagtattacgaccacattgtgccttgtgggctggcggatgcgggtgtcacgacgctcaccgaggagctggggcgcgatgttagtgtttctgacgcctactcatccctcgcccacaacctcgttgatgctttgaacggcgacttgccggtgcattcctag

SEQ ID NO: 20 - protein, 252 aa, Corynebacterium jeikeium
MGFQQGSIRKANMGTTGTNDGATTPPANTSTPAVDIDVRDLGTVDYEDTWHLQANLAAQRAEEKIPDTILLLQHPPTYTAGKRTQDSDRPTNGLPVVDVDRGGRITWHGPGQLVAYPIIKLADPVDVVDYVRRLEQALIQTCEDLGLHGTGRVEGRSGVWLPAGVINGELKPARKIAAIGIRVTRGVTMHGVALNCDNTMEYYDHIVPCGLADAGVTTLTEELGRDVSVSDAYSSLAHNLVDALNGDLPVHS

SEQ ID NO: 21 - DNA, 7070 bp, artificial
  Plasmid pH273
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctcctctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 22 - DNA, 7070 bp, artificial; identical to SEQ ID NO: 21
  Plasmid pH373

SEQ ID NO: 23 - DNA, 8766 bp, artificial
  Plasmid pH304
tcgagaggcctgacgtcgggcccggtaccacgcgtcatatgactagttcggacctagggatatcgtcgacatcgatgctcttctgcgttaattaacaattgggatctctcaactaatgcagcgatgcgttctttccagaatgctttcatgacagggatgctgtcttgatcaggcaggcgtctgtgctggatgccgaagctggatttattgtcgcctttggaggtgaagttgacgctcactcgagaatcatcggccaaccatttggcattgaatgttctaggttcggaggcggaggttttctcaattagtgcgggatcgagccactgcgcccgcaggtcatcgtctccgaagagcttccacactttttcgaccggcaggttaagggttttggaggcattggccgcgaacccatcgctggtcatcccgggtttgcgcatgccacgttcgtattcataaccaatcgcgatgccttgagcccaccagccactgacatcaaagttgtccacgatgtgctttgcgatgtgggtgtgagtccaagaggtggcttttacgtcgtcaagcaattttagccactcttcccacggctttccggtgccgttgaggatagcttcaggggacatgcctggtgttgagccttgcggagtggagtcagtcatgcgaccgagactagtggcgctttgggtaccgggccccccctcgaggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatcatgtcgacttcagttacttcaccagcccacaacaacgcacattcctccgaatttttggatgcgttggcaaaccatgtgttgatcggcgacggcgccatgggcacccagctccaaggctttgacctggacgtggaaaaggatttccttgatctggaggggtgtaatgagattctcaacgacacccgccctgatgtgttgaggcagattcaccgcgcctactttgaggcgggagctgacttggttgagaccaatacttttggttgcaacctgccgaacttggcggattatgacatcgctgatcgttgccgtgagcttgcctacaagggcactgcagtggctagggaagtggctgatgagatggggccgggccgaaacggcatgcggcgtttcgtggttggttccctgggacctggaacgaagcttccatcgctgggccatgcaccgtatgcagatttgcgtgggcactacaaggaagcagcgcttggcatcatcgacggtggtggcgatgcctttttgattgagactgctcaggacttgcttcaggtcaaggctgcggttcacggcgttcaagatgccatggctgaacttgatacattcttgcccattatttgccacgtcaccgtagagaccaccggcaccatgctcatgggttctgagatcggtgccgcgttgacagcgctgcagccactgggtatcgacatgattggtctgaactgcgccaccggcccagatgagatgagcgagcacctgcgttacctgtccaagcacgccgatattcctgtgtcggtgatgcctaacgcaggtcttcctgtcctgggtaaaaacggtgcagaatacccacttgaggctgaggatttggcgcaggcgctggctggattcgtctccgaatatggcctgtccatggtgggtggttgttgtggcaccacacctgagcacatccgtgcggtccgcgatgcggtggttggtgttccagagcaggaaacctccacactgaccaagatccctgcaggccctgttgagcaggcctcccgcgaggtggagaaagaggactccgtcgcgtcgctgtacacctcggtgccattgtcccaggaaaccggcatttccatgatcggtgagcgcaccaactccaacggttccaaggcattccgtgaggcaatgctgtctggcgattgggaaaagtgtgtggatattgccaagcagcaaacccgcgatggtgcacacatgctggatctttgtgtggattacgtgggacgagacggcaccgccgatatggcgaccttggcagcacttcttgctaccagctccactttgccaatcatgattgactccaccgagccagaggttattcgcacaggccttgagcacttgggtggacgaagcatcgttaactccgtcaactttgaagacggcgatggccctgagtcccgctaccagcgcatcatgaaactggtaaagcagcacggtgcggccgtggttgcgctgaccattgatgaggaaggccaggcacgtaccgctgagcacaaggtgcgcattgctaaacgactgattgacgatatcaccggcagctacggcctggatatcaaagacatcgttgtggactgcctgaccttcccgatctctactggccaggaagaaaccaggcgagatggcattgaaaccatcgaagccatccgcgagctgaagaagctctacccagaaatccacaccaccctgggtctgtccaatatttccttcggcctgaaccctgctgcacgccaggttcttaactctgtgttcctcaatgagtgcattgaggctggtctggactctgcgattgcgcacagctccaagattttgccgatgaaccgcattgatgatcgccagcgcgaagtggcgttggatatggtctatgatcgccgcaccgaggattacgatccgctgcaggaattcatgcagctgtttgagggcgtttctgctgccgatgccaaggatgctcgcgctgaacagctggccgctatgcctttgtttgagcgtttggcacagcgcatcatcgacggcgataagaatggccttgaggatgatctggaagcaggcatgaaggagaagtctcctattgcgatcatcaacgaggaccttctcaacggcatgaagaccgtgggtgagctgtttggttccggacagatgcagctgccattcgtgctgcaatcggcagaaaccatgaaaactgcggtggcctatttggaaccgttcatggaagaggaagcagaagctaccggatctgcgcaggcagagggcaagggcaaaatcgtcgtggccaccgtcaagggtgacgtgcacgatatcggcaagaacttggtggacatcattttgtccaacaacggttacgacgtggtgaacttgggcatcaagcagccactgtccgccatgttggaagcagcggaagaacacaaagcagacgtcatcggcatgtcgggacttcttgtgaagtccaccgtggtgatgaaggaaaaccttgaggagatgaacaacgccggcgcatccaattacccagtcattttgggtggcgctgcgctgacgcgtacctacgtggaaaacgatctcaacgaggtgtacaccggtgaggtgtactacgcccgtgatgctttcgagggcctgcgcctgatggatgaggtgatggcagaaaagcgtggtgaaggacttgatcccaactcaccagaagctattgagcaggcgaagaagaaggcggaacgtaaggctcgtaatgagcgttcccgcaagattgccgcggagcgtaaagctaatgcggctcccgtgattgttccggagcgttctgatgtctccaccgatactccaaccgcggcaccaccgttctggggaacccgcattgtcaagggtctgcccttggcggagttcttgggcaaccttgatgagcgcgccttgttcatggggcagtggggtctgaaatccacccgcggcaacgagggtccaagctatgaggatttggtggaaactgaaggccgaccacgcctgcgctactggctggatcgcctgaagtctgagggcattttggaccacgtggccttggtgtatggctacttcccagcggtcgcggaaggcgatgacgtggtgatcttggaatccccggatccacacgcagccgaacgcatgcgctttagcttcccacgccagcagcgcggcaggttcttgtgcatcgcggatttcattcgcccacgcgagcaagctgtcaaggacggccaagtggacgtcatgccattccagctggtcaccatgggtaatcctattgctgatttcgccaacgagttgttcgcagccaatgaataccgcgagtacttggaagttcacggcatcggcgtgcagctcaccgaagcattggccgagtactggcactcccgagtgcgcagcgaactcaagctgaacgacggtggatctgtcgctgattttgatccagaagacaagaccaagttcttcgacctggattaccgcggcgcccgcttctcctttggttacggttcttgccctgatctggaagaccgcgcaaagctggtggaattgctcgagccaggccgtatcggcgtggagttgtccgaggaactccagctgcacccagagcagtccacagacgcgtttgtgctctaccacccagaggcaaagtactttaacgtctaatctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 24 - DNA, 7070 bp, artificial
  Plasmid pH399
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctccgctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 25 - DNA, 6625 bp, artificial
  Plasmid pH484
tcgagaggcctgacgtcgggcccggtaccgttgctcgctgatctttcggcttaacaactttgtattcaatcagtcgggcatagaaagaaaacgcaatgatataggaaccaactgccgccaaaaccagccacacagagttgattgtttcgccacgggagaaagcgattgctccccaacccaccgccgcgataaccccaaagacaaggagaccaacgcgggcggtcggtgacattttaggggacttcttcacgcctactggaaggtcagtagcgttgctgtacaccaaatcatcgtcattgatgttgtcagtctgttttatggtcacgatctttactgttttctcttcgggtcgtttcaaagccactatgcgtagaaacagcgggcagaaacagcgggcagaaactgtgtgcagaaatgcatgcagaaaaaggaaagttcggccagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagacatatgcggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacaatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 26 - DNA, 363 bp, artificial
  Sequence of the promotor P497_P1284 = PgrESPEFTu>P497_P1284
cggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacataca

SEQ ID NO: 27 - DNA, 6350 bp, artificial
  Plasmid pH491
tcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgagagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaacatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 28 - DNA, 5477 bp, artificial
  Plasmid pH429
tcgagctctccaatctccactgaggtacttaatccttccggggaattcgggcgcttaaatcgagaaattaggccatcaccttttaataacaatacaatgaataattggaataggtcgacacctttggagcggagccggttaaaattggcagcattcaccgaaagaaaaggagaaccacatgcttgccctaggttggattacatggatcattattggtggtctagctggttggattgcctccaagattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattaaaactatacgaaccggtttgtctatattggtgttagacagttcgtcgtatcttgaaacagaccaacccgaaaggacgtggccgaacgtggctgctagctaatccttgatggtggacttgctggatctcgattggtccacaacatcagtcctcttgagacggctcgcgatttggctcggcagttgttgtcggctccacctgcggactactcaatttagtttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttctttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacgcgtcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 29 - DNA, 5697 bp, artificial
  Plasmid pH449
tcgaggcgtcttccggtgtcatggttgaaccgaattccagcacaatattttccggtttaaagcaatcgatcacatagtcgattttgtccaaccactgaaaacctgcaaggaccacccaatcccctgcagcatgttcagcaaccattggcagcggcggatagcgaacttcccccttttctcccgttgccattttcgcgtcactgatcaggtgactgagctttttgtagccttccggatttttacacaagactgtcaacacgccttcttgcagactcagctccgcaccataaacggtatgcattccagcttccgcggcagcttccgcaaatctcactgcaccataaaaaccatccctatccatgactgatagagcaacaagtcctaactttttggcctgcacaaccacatcagacggatccgatgcgccagtgagaaagttataactgctggtggcatgcagctcggcaaaaggaaccgacgcttccccctgcatggcagatgaaggcgcctgcgcatccggctcatgcagcaccggacgcagagattcgacctttttacctgagaggattctttccaatttggaccacgataatggcctgccgttaaagcttcccccgccattccattccataatgataggatacatttttagaacaaattttccaataagttttccacgccagccggagaaggaaatagaccaagctgtacagatcgacgcgtcctggctgagtacaacgtcggctccggcgcagacctcaccccagttggctccagcgaaatcgtgccactggcactattctggaaggaccacgactccatcgacggcattgacggcgagtccgttgccatccctaacgatccttccaaccagggccgcgccatcaacgttctcgttcaggcaggtctggtcaccctgaagaccccaggtctggtcaccccagctccagtcgatatcgacgaggcagcttccaaggtttccgtcatcccagtcgacgcagctcaggcaccaaccgcttaccaggagggtcgcccagcgatcatcaacaactccttccttgaccgcgcaggcatcgatccaaacctcgcggtcttcgaagatgatcctgagtctgaagaagcagagccatacatcaacgtcttcgtcaccaaggctgaggacaaggacgatgccaacatcgcccgcctcgttgagctgtggcacgacccagaggttctggctgcagtagaccgcgactctgagggcacctccgtcccagttgatcgtccaggagctgaccttcaggaaatccttgatcgccttgaggctgatcaggaaaacgcataatctcttttgagttctttgcatacccatgtgcagatttctttgcacaatcacagcctgaaaatcagactgtgaacttcaaacgcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 30 - DNA, 7318 bp, artificial
  Plasmid pOM427
ggccgctcgatttaaatctcgagctctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccaatccgcaagctccaccgactcgttggcgtgcgactctagataaatatcaagcagctggccgccaataacctcagtacgcatgccacgccaagcatccctcgtgcgggccaatgcctctgcactcaaaccggaatcctgcagcatgtcttctgcccacaccaatgccatatcgccagccaaaatcgagactgaaacgccaaagtgctcgggatcgccttcgaaattattggcgcggtgatcagcttccacagcccggtgaactgtgggggctccgcgccgggtatcagaagaatcgataatatcgtcatgaatcaaggcacaagcctggatgaattcgagactcgctgcggcgtcaaggacggactcaagtttttcagaagaattcttatggccttgcgccgccaggaaaccagcccacgcataaagaggacggattcgctttcctccattgagcacgaaactgcgaagatgggccacagcatctgtgacaggagcgccgatatcagcaattgttagctcttgagcatcgaggaactgcgtcaaacgatctcgcacgacctccggaaatttgtcgaggtcaaggtcatgggcatcgaaactgctcaaggagacgtccttcaatcgaatagggggatgcgggctgaattttggtggaggtgaataaatgccagaggcagtcccaacaaaacactctcatcacactaagatacccgtcgactcatacgttaaatctatcaccgcaagggataaatatctaacaccgtgcgtgttgactattttacctctggcggtgataatggttgcatgtactaaggaggattaattaatgtccctaacgaacatcccagcctcatctcaatgggcaattagcgacgttttgaagcgtccttcacccggccgagtacctttttctgtcgagtttatgccaccccgcgacgatgcagctgaagagcgtctttaccgcgcagcagaggtcttccatgacctcggtgcatcgtttgtctccgtgacttatggtgctggcggatcaacccgtgagagaacctcacgtattgctcgacgattagcgaaacaaccgttgaccactctggtgcacctgaccctggttaaccacactcgcgaagagatgaaggcaattcttcgggaatacctagagctgggattaacaaacctgttggcgcttcgaggagatccgcctggagacccattaggcgattgggtgagcaccgatggaggactgaactatgcctctgagctcatcgatcttattaagtccactcctgagttccgggaattcgacctcggtatcgcctccttccccgaagggcatttccgggcgaaaactctagaagaagacaccaaatacactctggcgaagctgcgtggaggggcagagtactccatcacgcagatgttctttgatgtggaagactacctgcgacttcgtgatcgccggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggc

SEQ ID NO: 31 - DNA, 5715 bp, artificial
  Plasmid pCLIK5APsodTKT
cgcgtcggcaaattagtcgaatgaagttaattaaaagttcccgaatcaatctttttaatgttttcaaaccatttgaaggtgtgctgacccaggtggacgccaacctttaaaaagcttcagacttttatttccacttcataaaaactgcctgtgacgattccgttaaagattgtgccaaatcactgcgcaaaactcgcgcggaaccagaccttgccatgctatcgcctattcacactatttgagtaatcggaaatagatgggtgtagacgcttgattggcggacggttcacagcggacgatttcaggccctcgtagctcgagagtttgaaggggtccgattcgttccgttcgtgacgctttgtgaggttttttgacgttgcaccgtattgcttgccgaacatttttcttttcctttcggtttttcgagaattttcacctacaaaagcccacgtcacagctcccagacttaagattgatcacacctttgacacatttgaaccacagttggttataaaatgggttcaacatcactatggttagaggtgttgacgggtcagattaagcaaagactactttcggggtagatcacctttgccaaatttgaaccaattaacctaagtcgtagatctgatcatcggatctaacgaaaacgaaccaaaactttggtcccggtttaacccaggaaggatagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccttgaccaccttgacgctgtcacctgaacttcaggcgctcactgtacgcaattacccctctgattggtccgatgtggacaccaaggctgtagacactgttcgtgtcctcgctgcagacgctgtagaaaactgtggctccggccacccaggcaccgcaatgagcctggctccccttgcatacaccttgtaccagcgggttatgaacgtagatccacaggacaccaactgggcaggccgtgaccgcttcgttctttcttgtggccactcctctttgacccagtacatccagctttacttgggtggattcggccttgagatggatgacctgaaggctctgcgcacctgggattccttgaccccaggacaccctgagtaccgccacaccaagggcgttgagatcaccactggccctcttggccagggtcttgcatctgcagttggtatggccatggctgctcgtcgtgagcgtggcctattcgacccaaccgctgctgagggcgaatccccattcgaccaccacatctacgtcattgcttctgatgggtcgacatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtacca

SEQ ID NO: 32 - DNA, 5083 bp, artificial
  Plasmid pH626 int SacB delta sdaA
ctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtaccacgcgtgccgatcttctcaaaggacacgacggaaacggctaaattcgcggatctccgtttaaggcattgaagcatttggaggccccaagacatgacccagaccctgtaaagcgcttaaacggcgttttagagggtcatagttttgggacaagtgggacaagtgtgaatcctgaaagcttccagggcaaggatccaccacaaaccggccatcgccctttggaatcggtccgaaaattgcaggtacagagccttttaccgagaaaatccaccacagattgctgaaatttcgtgatctgtggtggattcgtgcaacttcagactcttacggaggcgatggaccaaaaacaactacaatcaagcagatcaccttgtacaccaccatagaaaaggcccaccctcagcccggtacggctttaacacggcttggattttgtcttgcttggcgaggtaggactggcactgggcgtcgataagctcagctgaccacccggtgacctgcgccattgcttcggccgtcgcacgcacggcagccggttgcacataacccagcgtgccgagcacgatgcgacgatccagcacgtccgccaggtcgacggccgcctcctcggcgacagcaaaaacggcctgcgccgcgatatcaaggttgtctgggtcaagtcggcgccccaggtcgggttgctttgcgacgagatccagcactttttcatgctcagttccatacagtctggccagatgcacgcggatttcatcatccacatccagctcggggtggctgcgaagcgctgactcaaaggaatcagccacggactcatacgcgccaaaagaagtactcaacggct

SEQ ID NO: 33 - DNA, 6995 bp, artificial
  Plasmid pOM253
aaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttgggtctagaggaggtgaaacaatgtcccagaatggccgtccagtagtcctcatcgccgataagcttgcgcagtccactgttgacgcgcttggagatgcagtagaagtccgttgggttgacggacctaaccgcccagaactgcttgatgcagttaaggaagcggacgcactgctcgtgcgttctgctaccactgtcgatgctgaagtcatcgccgctgcccctaacttgaagatcgtcggtcgtgccggcgtgggcttggacaacgttgacatccctgctgccactgaagctggcgtcatggttgctaacgcaccgacctctaatattcactccgcttgtgagcacgcaatttctttgctgctgtctactgctcgccagatccctgctgctgatgcgacgctgcgtgagggcgagtggaagcggtcttctttcaacggtgtggaaattttcggaaaaactgtcggtatcgtcggttttggccacattggtcagttgtttgctcagcgtcttgctgcgtttgagaccaccattgttgcttacgatccttacgctaaccctgctcgtgcggctcagctgaacgttgagttggttgagttggatgagctgatgagccgttctgactttgtcaccattcaccttcctaagaccaaggaaactgctggcatgtttgatgcgcagctccttgctaagtccaagaagggccagatcatcatcaacgctgctcgtggtggccttgttgatgagcaggctttggctgatgcgattgagtccggtcacatctgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaggagggattcatcatgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatagggggatcgatatccgtttaggctgggcggatccgccctcccgcacgctttgcgggagggcggtaccaggggtgcttctactgaagaggctcaggatcgtgcgggtactgacgttgctgattctgtgctcaaggcgctggctggcgagttcgtggcggatgctgtgaacgtttccggtggtcgcgtgggcgaagaggttgctgtgtggatggatctggctcgcaagcttggtcttcttgctggcaagcttgtcgacgccgccccagtctccattgaggttgaggctcgaggcgagctttcttccgagcaggtcgatgcacttggtttgtccgctgttcgtggtttgttctccggaattatcgaagagtccgttactttcgtcaacgctcctcgcattgctgaagagcgtggcctggacatctccgtgaagaccaactctgagtctgttactcaccgttccgtcctgcaggtcaaggtcattactggcagcggcgcgagcgcaactgttgttggtgccctgactggtcttgagcgcgttgagaagatcacccgcatcaatggccgtggcctggatctgcgcgcagagggtctgaacctcttcctgcagtacactgacgctcctggtgcactgggtaccgttggtaccaagctgggtgctgctggcatcaacatcgaggctgctgcgttgactcaggctgagaagggtgacggcgctgtcctgatcctgcgtgttgagtccgctgtctctgaagagctggaagctgaaatcaacgctgagttgggtgctacttccttccaggttgatcttgactaattagagatccatttgcttgaaccgccttcccatctttgaattcattcaaggtggtaaggcggttttcgctcttttaatacagttttaatggtagatttgggatccctc

SEQ ID NO: 34 - DNA, 11491 bp, artificial
  Plasmid pOM615
ttgatcagcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaaggaggtgatagtatgtcttctgcagctacccgccgtaattcagcccccttcgttcagcgccacatcggcccaaaccaggccgatacccaggagatcctcgattacctgggctatgaatcttccgccgcgctggccgacgatgccctcccgaagtcgatccgccaggcaggcccgatcggcctgccggaggcactggatgagacggacaccctggccgccctgcgtgcttacgctgacaagaacgtgcagaagcagcagctgatcggcaacggttacttcgacacgatcactccggccgtgattcgccgcaacgtggtggagaacccgggctggtacaccgcctacaccccctaccagccggaaatctcccaggggcgcctcgaggccctgctgaacttccagacgatggtgcaggacctgaccggcctgccagtggccggtgcttcgctgttagacgaagccaccgcagtggccgaggccgtgcagctgatggctcgcggcaatgcgaaggctgccaagaagggcggcgtggtgctgctggattcctccctgcaccagcagtccatcaccgtaaccctggcgcgcgctgaggctgcgggtatcccggtggaggtcgtggacctggacggcgaagatgctaccgccgcgtttgagggccgcgagaacctcgtcggcgtggtgctttccaaccccggctccaccggccgcgtccgcgacctgtccggtctgatctctgcggcgaaggagaccggcgctctggtgacggtcgcttgtgacctgctggctcaggttctggtgacctccccgggctcccaaggtgccgacattgctgtcggctccgcccagcgcttcggcgtgccgctattcttcggtggcccgcacgcgggcttcatctcctgcaccgaggctctgcagcgtaagctgccgggccgcatcgtgggcgtgtccgtggatgccgagggcaccccggcctaccgcttggctctgcagacccgtgagcagcacattcgccgcgacaaggccaccagtaacatctgtaccgctcaggctctgctggccgtcgttgccggtttctacgcggtctggcacggcccagccggcctgcgcgccatcgccgagggggtgcacgcccgcgcgaccgccctggccgttgccctgtccgaggctggcctgacgctggcgcacgataccttcttcgacaccgtcaccgttgacgtgtctggttcttctcttggtgacgcccccacggctctgcgcgccgcagccgaggcaggctacaacctgcgccaggttaacgattccttcgtcggcatctctgtcggcgagtccaccacggacgaggacattgccaagctgatcgaggtgctgggctcccgcaccggcgaggtcaactccgcgagcttcgacgtcaccgccggcccgctgggcgaggccggcgtgctgcgcgcggaggacgaggagattctgacccacccgatcttcaccgccatcacttccgagacccagatgatgcgctacatgcgcaagctggccgaccgcgacctggcgctggatcgtacgatgatcccgctgggctcctgcaccatgaagctgaacgcggccgtctccatggagccgatcacctggcctggcttcgcaggcatccacccgcacgtcccggccgagcaggcgcagggctggctggagctcatcgaggacctggaggagcgcctggcgaagatcaccggctacgccaaggtttccgtccagccgaacgcgggctcccagggcgagttcgccggcctgctggcgatccaccgctaccaccagtcccgcggcgacgatcagcgtgacatcgttctgatcccggcctccgcgcacggcaccaacgctgcctctgcagcgctggcgggcctgaaggtcgtggctgtgaagaatgccgaagacggctccatcgacgtgccggacctggaggccaagctggagaagtacggcgagcagaccgccgccatcatgctgacctacccctccacccacggcgtgttcgaggagcaggtgcgcgacgtctgccagaaggttcacgacgctggcggccaggtgtacgtcgacggcgctaacctgaacgccctggtcggcctggcccagccgggcgagttcggcggcgacgtatcccacctgaacctgcacaagaccttcaccatcccgcacggtggtggcggcccgggcgttggcccggtgtgcgtggcagagcacctgatcccgttcctgcccaccgacccgaacgccgacgttatcgagggcgatgctgccctgcagtccggccagccggtctccggcgcgcagtacggctccgctggcgtgctgccgatcacctggtcctacatcgcacagatgggcgacgaaggcctgactgaggcctcccgcatggccctggtgaacgccaactacgtttcccgcaagctggaggactactacccgacgctgtacaagggcgacaccggcctggtcgcccacgagtgcatcctggacctgcgtgaactcacgaaggcctccggcatcaccgccgaggacgtatccaagcgcctgatggacttcggcttccacgccccgaccttggccttcccggtcgccggcaccctgatgatggagcccaccgagtcggaggacaaggaagagctggaccgcttcatcgaagcgatgatcaccatccacggggagatccaggaagttatcgacggcaaggtcaccgccgaacagtccgttctgcgccacgcgccgtttaccgcctattctgttgtgcgggacgacttcgaggaagctgtatccggtggtcacttcagccgtgccaaggccgcatacccggtggctagcctgcgacacaccaagtacttcacccccgtgcgtcgtatcgacaacgcctatggcgaccgcaatctcgtgtgcacctgcccgcccctggaagacttcgcaattaacgaggactagggtccgctggagctaagggcgtcacaaagagaagaaggagaacaagacatgaccgaacttaagaagaccgcgctgcacctggtgcacgagaagttgggcgcgcgatttaccgacttcggcggctgggacatgcctctgaagtacagcagtgagctggacgagcaccacgctgtacgcaatgccgtgggcgtattcgacctctcccacatgggtgaggttcgcgtgaccggcccgcaggcagcggagttcctggaccacgcgctgatttcgaagctgtcggcagtgaaggtcggcaaggcgaagtactcgatgatctgcaccgaatccggtggcatcatcgacgacctgatcacctaccgcctgggcgacaacgagttcctgatcgtgccgaacgcgggcaacgtggacaacgtggtctccgcactgcagggccgcaccgagggctttgacgtggaggttaacaacgagtccgatgcgacctccatgatcgccgtacaggggcccaaggccgcgcaggcgatgctggagatcgtggagaacgtcgtggatgcacccgaggcatccggcgcgggcgagaccgttgccgaggctatcgaggggctgggttactacgcggcattcagcggtgttgccgcaggtcagcccgtgctggtggcccgcacaggctataccggcgaggacggtttcgagctgatcgtggctaacgatggtgcggagaccgtgtggaccaaggctatggaccaggctgcgcagctgggtggcctgccgtgtggcctggcctgccgcgacaccctgcgcctggaggctggcatgccgctgtacggcaacgagctatcgctgaagctcaccccggtcgatgctgggctgggcattcttgcggcgacgaagtctaaggactctttcgttggtcgtgacgccatcgtttccgccaaggaaaagggtacccagcaggtacttatcgggctggcgggcgagggtcgccgcgctgcccgtgggggatacgaggtgtttgccggtgacggcgagaaggccatcggtgccgtgacctccggtgcactgtcgccgacgctgggccacccggtggcattggcatacgtcgcgaagtccgcagtgtcctccggcgcggccgctgagggtgcgaccgtggaggtagacatccgcggcaagcgctttgaatacaaggttgtggcgctgccgttctactcccgcgagaagtaacgcaagaagtaacgaaaagggttgtctgcccgctggtgtaaggtttgggcagacaaccacagaagtaaccacacaaggtttcacaagagaggcttaaaacaatgactgcactgccaactgacttcctgtactccgaagagcacgagtgggttaacacctccgctgttgttgagggcgagaccgtgcgcgtgggcattacccacatcgccgctgaggcgctgggtgacatcgtgttcgtcgagctgccggaggttggctccgaggttgaggccggcgaggctttcggcgaggttgagtccaccaagtccgtttccgacatctacgcaccggtttctggcgaggttgtggctgtcaacgaggcgctggaagacaacgctggcctgatcaacgaagatccatacggtgagggctggctgtacgaggtcaaggtgaccgaggccggcgagctgatggaggctgaggcttaccaggcggctaacgagtaaaacaagaccctatggtcggttccatgttcgcattaatcctcggcttggccctgttgggccttgccgtgtggttactcctgcgagcaaaagatagcaccggatccgccctcccgcacgctttgcgggagggcttttcttttcccggtatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaaccgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgac

SEQ ID NO: 35 - DNA, 11639 bp, artificial
  Plasmid pOM616
ttgatcagcggccgcttcgcgaagcttgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaaggaggtgatagtatgtcttctgcagctacccgccgtaattcagcccccttcgttcagcgccacatcggcccaaaccaggccgatacccaggagatcctcgattacctgggctatgaatcttccgccgcgctggccgacgatgccctcccgaagtcgatccgccaggcaggcccgatcggcctgccggaggcactggatgagacggacaccctggccgccctgcgtgcttacgctgacaagaacgtgcagaagcagcagctgatcggcaacggttacttcgacacgatcactccggccgtgattcgccgcaacgtggtggagaacccgggctggtacaccgcctacaccccctaccagccggaaatctcccaggggcgcctcgaggccctgctgaacttccagacgatggtgcaggacctgaccggcctgccagtggccggtgcttcgctgttagacgaagccaccgcagtggccgaggccgtgcagctgatggctcgcggcaatgcgaaggctgccaagaagggcggcgtggtgctgctggattcctccctgcaccagcagtccatcaccgtaaccctggcgcgcgctgaggctgcgggtatcccggtggaggtcgtggacctggacggcgaagatgctaccgccgcgtttgagggccgcgagaacctcgtcggcgtggtgctttccaaccccggctccaccggccgcgtccgcgacctgtccggtctgatctctgcggcgaaggagaccggcgctctggtgacggtcgcttgtgacctgctggctcaggttctggtgacctccccgggctcccaaggtgccgacattgctgtcggctccgcccagcgcttcggcgtgccgctattcttcggtggcccgcacgcgggcttcatctcctgcaccgaggctctgcagcgtaagctgccgggccgcatcgtgggcgtgtccgtggatgccgagggcaccccggcctaccgcttggctctgcagacccgtgagcagcacattcgccgcgacaaggccaccagtaacatctgtaccgctcaggctctgctggccgtcgttgccggtttctacgcggtctggcacggcccagccggcctgcgcgccatcgccgagggggtgcacgcccgcgcgaccgccctggccgttgccctgtccgaggctggcctgacgctggcgcacgataccttcttcgacaccgtcaccgttgacgtgtctggttcttctcttggtgacgcccccacggctctgcgcgccgcagccgaggcaggctacaacctgcgccaggttaacgattccttcgtcggcatctctgtcggcgagtccaccacggacgaggacattgccaagctgatcgaggtgctgggctcccgcaccggcgaggtcaactccgcgagcttcgacgtcaccgccggcccgctgggcgaggccggcgtgctgcgcgcggaggacgaggagattctgacccacccgatcttcaccgccatcacttccgagacccagatgatgcgctacatgcgcaagctggccgaccgcgacctggcgctggatcgtacgatgatcccgctgggctcctgcaccatgaagctgaacgcggccgtctccatggagccgatcacctggcctggcttcgcaggcatccacccgcacgtcccggccgagcaggcgcagggctggctggagctcatcgaggacctggaggagcgcctggcgaagatcaccggctacgccaaggtttccgtccagccgaacgcgggctcccagggcgagttcgccggcctgctggcgatccaccgctaccaccagtcccgcggcgacgatcagcgtgacatcgttctgatcccggcctccgcgcacggcaccaacgctgcctctgcagcgctggcgggcctgaaggtcgtggctgtgaagaatgccgaagacggctccatcgacgtgccggacctggaggccaagctggagaagtacggcgagcagaccgccgccatcatgctgacctacccctccacccacggcgtgttcgaggagcaggtgcgcgacgtctgccagaaggttcacgacgctggcggccaggtgtacgtcgacggcgctaacctgaacgccctggtcggcctggcccagccgggcgagttcggcggcgacgtatcccacctgaacctgcacaagaccttcaccatcccgcacggtggtggcggcccgggcgttggcccggtgtgcgtggcagagcacctgatcccgttcctgcccaccgacccgaacgccgacgttatcgagggcgatgctgccctgcagtccggccagccggtctccggcgcgcagtacggctccgctggcgtgctgccgatcacctggtcctacatcgcacagatgggcgacgaaggcctgactgaggcctcccgcatggccctggtgaacgccaactacgtttcccgcaagctggaggactactacccgacgctgtacaagggcgacaccggcctggtcgcccacgagtgcatcctggacctgcgtgaactcacgaaggcctccggcatcaccgccgaggacgtatccaagcgcctgatggacttcggcttccacgccccgaccttggccttcccggtcgccggcaccctgatgatggagcccaccgagtcggaggacaaggaagagctggaccgcttcatcgaagcgatgatcaccatccacggggagatccaggaagttatcgacggcaaggtcaccgccgaacagtccgttctgcgccacgcgccgtttaccgcctattctgttgtgcgggacgacttcgaggaagctgtatccggtggtcacttcagccgtgccaaggccgcatacccggtggctagcctgcgacacaccaagtacttcacccccgtgcgtcgtatcgacaacgcctatggcgaccgcaatctcgtgtgcacctgcccgcccctggaagacttcgcaattaacgaggactagggtccgctggagctaagggcgtcacaaagagaagaaggagaacaagacatgaccgaacttaagaagaccgcgctgcacctggtgcacgagaagttgggcgcgcgatttaccgacttcggcggctgggacatgcctctgaagtacagcagtgagctggacgagcaccacgctgtacgcaatgccgtgggcgtattcgacctctcccacatgggtgaggttcgcgtgaccggcccgcaggcagcggagttcctggaccacgcgctgatttcgaagctgtcggcagtgaaggtcggcaaggcgaagtactcgatgatctgcaccgaatccggtggcatcatcgacgacctgatcacctaccgcctgggcgacaacgagttcctgatcgtgccgaacgcgggcaacgtggacaacgtggtctccgcactgcagggccgcaccgagggctttgacgtggaggttaacaacgagtccgatgcgacctccatgatcgccgtacaggggcccaaggccgcgcaggcgatgctggagatcgtggagaacgtcgtggatgcacccgaggcatccggcgcgggcgagaccgttgccgaggctatcgaggggctgggttactacgcggcattcagcggtgttgccgcaggtcagcccgtgctggtggcccgcacaggctataccggcgaggacggtttcgagctgatcgtggctaacgatggtgcggagaccgtgtggaccaaggctatggaccaggctgcgcagctgggtggcctgccgtgtggcctggcctgccgcgacaccctgcgcctggaggctggcatgccgctgtacggcaacgagctatcgctgaagctcaccccggtcgatgctgggctgggcattcttgcggcgacgaagtctaaggactctttcgttggtcgtgacgccatcgtttccgccaaggaaaagggtacccagcaggtacttatcgggctggcgggcgagggtcgccgcgctgcccgtgggggatacgaggtgtttgccggtgacggcgagaaggccatcggtgccgtgacctccggtgcactgtcgccgacgctgggccacccggtggcattggcatacgtcgcgaagtccgcagtgtcctccggcgcggccgctgagggtgcgaccgtggaggtagacatccgcggcaagcgctttgaatacaaggttgtggcgctgccgttctactcccgcgagaagtaacgcaagaagtaacgaaaagggttgtctgcccgctggtgtaaggtttgggcagacaaccacagaagtaaccacacaaggtttcacaagagaggcttaaaacaatgactgcactgccaactgacttcctgtactccgaagagcacgagtgggttaacacctccgctgttgttgagggcgagaccgtgcgcgtgggcattacccacatcgccgctgaggcgctgggtgacatcgtgttcgtcgagctgccggaggttggctccgaggttgaggccggcgaggctttcggcgaggttgagtccaccaagtccgtttccgacatctacgcaccggtttctggcgaggttgtggctgtcaacgaggcgctggaagacaacgctggcctgatcaacgaagatccatacggtgagggctggctgtacgaggtcaaggtgaccgaggccggcgagctgatggaggctgaggcttaccaggcggctaacgagtaaaacaagaccctatggtcggttccatgttcgcattaatcctcggcttggccctgttgggccttgccgtgtggttactcctgcgagcaaaagatagcaccggatccgccctcccgcacgctttgcgggagggcttttcttttcccggtatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaaccgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgac

SEQ ID NO: 36 - DNA, 13915 bp, artificial
  Plasmid pOM620AF
ttgatcagcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaaggaggtgatagtatgtcttctgcagctacccgccgtaattcagcccccttcgttcagcgccacatcggcccaaaccaggccgatacccaggagatcctcgattacctgggctatgaatcttccgccgcgctggccgacgatgccctcccgaagtcgatccgccaggcaggcccgatcggcctgccggaggcactggatgagacggacaccctggccgccctgcgtgcttacgctgacaagaacgtgcagaagcagcagctgatcggcaacggttacttcgacacgatcactccggccgtgattcgccgcaacgtggtggagaacccgggctggtacaccgcctacaccccctaccagccggaaatctcccaggggcgcctcgaggccctgctgaacttccagacgatggtgcaggacctgaccggcctgccagtggccggtgcttcgctgttagacgaagccaccgcagtggccgaggccgtgcagctgatggctcgcggcaatgcgaaggctgccaagaagggcggcgtggtgctgctggattcctccctgcaccagcagtccatcaccgtaaccctggcgcgcgctgaggctgcgggtatcccggtggaggtcgtggacctggacggcgaagatgctaccgccgcgtttgagggccgcgagaacctcgtcggcgtggtgctttccaaccccggctccaccggccgcgtccgcgacctgtccggtctgatctctgcggcgaaggagaccggcgctctggtgacggtcgcttgtgacctgctggctcaggttctggtgacctccccgggctcccaaggtgccgacattgctgtcggctccgcccagcgcttcggcgtgccgctattcttcggtggcccgcacgcgggcttcatctcctgcaccgaggctctgcagcgtaagctgccgggccgcatcgtgggcgtgtccgtggatgccgagggcaccccggcctaccgcttggctctgcagacccgtgagcagcacattcgccgcgacaaggccaccagtaacatctgtaccgctcaggctctgctggccgtcgttgccggtttctacgcggtctggcacggcccagccggcctgcgcgccatcgccgagggggtgcacgcccgcgcgaccgccctggccgttgccctgtccgaggctggcctgacgctggcgcacgataccttcttcgacaccgtcaccgttgacgtgtctggttcttctcttggtgacgcccccacggctctgcgcgccgcagccgaggcaggctacaacctgcgccaggttaacgattccttcgtcggcatctctgtcggcgagtccaccacggacgaggacattgccaagctgatcgaggtgctgggctcccgcaccggcgaggtcaactccgcgagcttcgacgtcaccgccggcccgctgggcgaggccggcgtgctgcgcgcggaggacgaggagattctgacccacccgatcttcaccgccatcacttccgagacccagatgatgcgctacatgcgcaagctggccgaccgcgacctggcgctggatcgtacgatgatcccgctgggctcctgcaccatgaagctgaacgcggccgtctccatggagccgatcacctggcctggcttcgcaggcatccacccgcacgtcccggccgagcaggcgcagggctggctggagctcatcgaggacctggaggagcgcctggcgaagatcaccggctacgccaaggtttccgtccagccgaacgcgggctcccagggcgagttcgccggcctgctggcgatccaccgctaccaccagtcccgcggcgacgatcagcgtgacatcgttctgatcccggcctccgcgcacggcaccaacgctgcctctgcagcgctggcgggcctgaaggtcgtggctgtgaagaatgccgaagacggctccatcgacgtgccggacctggaggccaagctggagaagtacggcgagcagaccgccgccatcatgctgacctacccctccacccacggcgtgttcgaggagcaggtgcgcgacgtctgccagaaggttcacgacgctggcggccaggtgtacgtcgacggcgctaacctgaacgccctggtcggcctggcccagccgggcgagttcggcggcgacgtatcccacctgaacctgcacaagaccttcaccatcccgcacggtggtggcggcccgggcgttggcccggtgtgcgtggcagagcacctgatcccgttcctgcccaccgacccgaacgccgacgttatcgagggcgatgctgccctgcagtccggccagccggtctccggcgcgcagtacggctccgctggcgtgctgccgatcacctggtcctacatcgcacagatgggcgacgaaggcctgactgaggcctcccgcatggccctggtgaacgccaactacgtttcccgcaagctggaggactactacccgacgctgtacaagggcgacaccggcctggtcgcccacgagtgcatcctggacctgcgtgaactcacgaaggcctccggcatcaccgccgaggacgtatccaagcgcctgatggacttcggcttccacgccccgaccttggccttcccggtcgccggcaccctgatgatggagcccaccgagtcggaggacaaggaagagctggaccgcttcatcgaagcgatgatcaccatccacggggagatccaggaagttatcgacggcaaggtcaccgccgaacagtccgttctgcgccacgcgccgtttaccgcctattctgttgtgcgggacgacttcgaggaagctgtatccggtggtcacttcagccgtgccaaggccgcatacccggtggctagcctgcgacacaccaagtacttcacccccgtgcgtcgtatcgacaacgcctatggcgaccgcaatctcgtgtgcacctgcccgcccctggaagacttcgcaattaacgaggactagggtccgctggagctaagggcgtcacaaagagaagaaggagaacaagacatgaccgaacttaagaagaccgcgctgcacctggtgcacgagaagttgggcgcgcgatttaccgacttcggcggctgggacatgcctctgaagtacagcagtgagctggacgagcaccacgctgtacgcaatgccgtgggcgtattcgacctctcccacatgggtgaggttcgcgtgaccggcccgcaggcagcggagttcctggaccacgcgctgatttcgaagctgtcggcagtgaaggtcggcaaggcgaagtactcgatgatctgcaccgaatccggtggcatcatcgacgacctgatcacctaccgcctgggcgacaacgagttcctgatcgtgccgaacgcgggcaacgtggacaacgtggtctccgcactgcagggccgcaccgagggctttgacgtggaggttaacaacgagtccgatgcgacctccatgatcgccgtacaggggcccaaggccgcgcaggcgatgctggagatcgtggagaacgtcgtggatgcacccgaggcatccggcgcgggcgagaccgttgccgaggctatcgaggggctgggttactacgcggcattcagcggtgttgccgcaggtcagcccgtgctggtggcccgcacaggctataccggcgaggacggtttcgagctgatcgtggctaacgatggtgcggagaccgtgtggaccaaggctatggaccaggctgcgcagctgggtggcctgccgtgtggcctggcctgccgcgacaccctgcgcctggaggctggcatgccgctgtacggcaacgagctatcgctgaagctcaccccggtcgatgctgggctgggcattcttgcggcgacgaagtctaaggactctttcgttggtcgtgacgccatcgtttccgccaaggaaaagggtacccagcaggtacttatcgggctggcgggcgagggtcgccgcgctgcccgtgggggatacgaggtgtttgccggtgacggcgagaaggccatcggtgccgtgacctccggtgcactgtcgccgacgctgggccacccggtggcattggcatacgtcgcgaagtccgcagtgtcctccggcgcggccgctgagggtgcgaccgtggaggtagacatccgcggcaagcgctttgaatacaaggttgtggcgctgccgttctactcccgcgagaagtaacgcaagaagtaacgaaaagggttgtctgcccgctggtgtaaggtttgggcagacaaccacagaagtaaccacacaaggtttcacaagagaggcttaaaacaatgactgcactgccaactgacttcctgtactccgaagagcacgagtgggttaacacctccgctgttgttgagggcgagaccgtgcgcgtgggcattacccacatcgccgctgaggcgctgggtgacatcgtgttcgtcgagctgccggaggttggctccgaggttgaggccggcgaggctttcggcgaggttgagtccaccaagtccgtttccgacatctacgcaccggtttctggcgaggttgtggctgtcaacgaggcgctggaagacaacgctggcctgatcaacgaagatccatacggtgagggctggctgtacgaggtcaaggtgaccgaggccggcgagctgatggaggctgaggcttaccaggcggctaacgagtaaaacaagaccctatggtcggttccatgttcgcattaatcctcggcttggccctgttgggccttgccgtgtggttactcctgcgagcaaaagatagcaccggatccgccctcccgcacgctttgcgggagggcttttcttttcccggtatttcagtagctcgggggcgtctcattcagcaaacctagctccatatccgcctctgggctctcccaccccgcaccaaaatctctcgcgagaggccttcagggcccttaaatgtcggaaaagttgcaatttgttaatgctactggttttcaaatatgcaaatgcgcaggtcaaggggcttttgggaaagtggaaaccaatcccgatttgaagaattgcaacttttccgacattaagcggccttcaggaagtgccaaacaccggaggcaccagcatcaacgtcaccacctggcatatctagcaccatcagaagcttcagcctcactacaggcggaacctgacgccagcgggacctcacccagcgggacagaacccagcggaacttggcgccagcggaaccgcagccaccagctcccaccagcacccagcagcgccaaggcaccactggagtatcatcgccaccatgggattccagcaaggcagcatccgcaaagcaaacatgggcacaaccggcactaacgacggggcaaccaccccaccagccaacaccagcaccccagccgtagacatcgacgtccgcgacctgggcaccgtggactacgaggacacttggcacctccaggcaaatctcgccgcccagcgcgccgaagaaaaaatcccagacaccatcctcctactccagcatccgccgacgtacaccgccggcaagcgcacccaggattccgaccgccctaccaacggcctgccagtagtcgacgtcgaccgcggaggccgcatcacctggcatggacccggccagttggtcgcatatcccatcatcaagctggccgacccggtggacgtggtcgattatgtccgccgcctggagcaggcgctaatccagacttgtgaagatcttggcctgcacggcaccggccgcgtagaggggcgttcgggcgtgtggctgcctgcgggcgtcattaatggcgagctcaagcccgcacgtaagatagccgcgatcggcatccgcgtgacgcgcggcgtgaccatgcacggagtggccctcaactgcgataacaccatggagtattacgaccacattgtgccttgtgggctggcggatgcgggtgtcacgacgctcaccgaggagctggggcgcgatgttagtgtttctgacgcctactcatccctcgcccacaacctcgttgatgctttgaacggcgacttgccggtgcattcctaggggaaaacccgaggtggattcctatacggcacccagcgtaggctagtcggcatgagtgtgaccgcagatggacgccggatgctacgcatcgaggcgaagaatgccgaaacccccatcgagtcgaagcctcggtggatccgcacgaccgcgaaggtcggcccggagtatcgggatattaagaatcgcgtgaagggcgctggcctgcacaccgtgtgccaagaggctggctgcccgaatatcaacgagtgctgggaggaccgcgaggcgacgttcctcatcggcggtgatacgtgttcccgccgttgcgacttctgccagattaagtccggccgcccgtccccgctggatatggacgagccacgccgcgtggcggagaatgtccgcgagatgggtctgcgctacgccaccatcaccggcgtgacgcgcgatgacctggatgatgagggcgcgtggctgtatgccgaggttgtgaagaagattcacgagctgaacccgaacacgggtgtggagaatctgacgccagatttttccaaccgcccggagctgctgaaggtcgtcttcgattcccagccggaggtgtttgcccacaacctggagacagttccgcgcatctttaagcgcatccgcccggcctttaagtacgaccgttccctggaggtcattcaggcagctcacgattacggcctggtgacgaagtccaacctgatcctgggcatgggtgagaagaaggaagaggtccgcgcggctatcaaggacctggcagacgccggcaccgacattctgacgattacccagtacctgcgcccgtcttccatgcaccacccgattgagcgttgggtgaagccggaggagttcatggagcactccgacgcagcctacgagctgggcatcaaggctgttatgtccggcccgttggtgcgttcctcttaccgcgccggccgcctgtacgcgcaggccaagcaggcgcgcggcgaggctattccggagaacctgaagcacttggaggagactctcgattccaccacgtcgcaggaggcctctacactgctggagcgctacggcgcttcggaggacacgccggtcactgcgtcgcgccgctagctcggcggggcactttcgccccgcctttattcatgccttattgttgaatccatggcgaaggataaaaaggacgtaagggtcaaggaagagaagaaggcagctaaggcaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaaccgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgac

SEQ ID NO: 37 - DNA, 14063 bp, artificial
  Plasmid pOM621AR
ttgatcagcggccgcttcgcgaagcttgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaaggaggtgatagtatgtcttctgcagctacccgccgtaattcagcccccttcgttcagcgccacatcggcccaaaccaggccgatacccaggagatcctcgattacctgggctatgaatcttccgccgcgctggccgacgatgccctcccgaagtcgatccgccaggcaggcccgatcggcctgccggaggcactggatgagacggacaccctggccgccctgcgtgcttacgctgacaagaacgtgcagaagcagcagctgatcggcaacggttacttcgacacgatcactccggccgtgattcgccgcaacgtggtggagaacccgggctggtacaccgcctacaccccctaccagccggaaatctcccaggggcgcctcgaggccctgctgaacttccagacgatggtgcaggacctgaccggcctgccagtggccggtgcttcgctgttagacgaagccaccgcagtggccgaggccgtgcagctgatggctcgcggcaatgcgaaggctgccaagaagggcggcgtggtgctgctggattcctccctgcaccagcagtccatcaccgtaaccctggcgcgcgctgaggctgcgggtatcccggtggaggtcgtggacctggacggcgaagatgctaccgccgcgtttgagggccgcgagaacctcgtcggcgtggtgctttccaaccccggctccaccggccgcgtccgcgacctgtccggtctgatctctgcggcgaaggagaccggcgctctggtgacggtcgcttgtgacctgctggctcaggttctggtgacctccccgggctcccaaggtgccgacattgctgtcggctccgcccagcgcttcggcgtgccgctattcttcggtggcccgcacgcgggcttcatctcctgcaccgaggctctgcagcgtaagctgccgggccgcatcgtgggcgtgtccgtggatgccgagggcaccccggcctaccgcttggctctgcagacccgtgagcagcacattcgccgcgacaaggccaccagtaacatctgtaccgctcaggctctgctggccgtcgttgccggtttctacgcggtctggcacggcccagccggcctgcgcgccatcgccgagggggtgcacgcccgcgcgaccgccctggccgttgccctgtccgaggctggcctgacgctggcgcacgataccttcttcgacaccgtcaccgttgacgtgtctggttcttctcttggtgacgcccccacggctctgcgcgccgcagccgaggcaggctacaacctgcgccaggttaacgattccttcgtcggcatctctgtcggcgagtccaccacggacgaggacattgccaagctgatcgaggtgctgggctcccgcaccggcgaggtcaactccgcgagcttcgacgtcaccgccggcccgctgggcgaggccggcgtgctgcgcgcggaggacgaggagattctgacccacccgatcttcaccgccatcacttccgagacccagatgatgcgctacatgcgcaagctggccgaccgcgacctggcgctggatcgtacgatgatcccgctgggctcctgcaccatgaagctgaacgcggccgtctccatggagccgatcacctggcctggcttcgcaggcatccacccgcacgtcccggccgagcaggcgcagggctggctggagctcatcgaggacctggaggagcgcctggcgaagatcaccggctacgccaaggtttccgtccagccgaacgcgggctcccagggcgagttcgccggcctgctggcgatccaccgctaccaccagtcccgcggcgacgatcagcgtgacatcgttctgatcccggcctccgcgcacggcaccaacgctgcctctgcagcgctggcgggcctgaaggtcgtggctgtgaagaatgccgaagacggctccatcgacgtgccggacctggaggccaagctggagaagtacggcgagcagaccgccgccatcatgctgacctacccctccacccacggcgtgttcgaggagcaggtgcgcgacgtctgccagaaggttcacgacgctggcggccaggtgtacgtcgacggcgctaacctgaacgccctggtcggcctggcccagccgggcgagttcggcggcgacgtatcccacctgaacctgcacaagaccttcaccatcccgcacggtggtggcggcccgggcgttggcccggtgtgcgtggcagagcacctgatcccgttcctgcccaccgacccgaacgccgacgttatcgagggcgatgctgccctgcagtccggccagccggtctccggcgcgcagtacggctccgctggcgtgctgccgatcacctggtcctacatcgcacagatgggcgacgaaggcctgactgaggcctcccgcatggccctggtgaacgccaactacgtttcccgcaagctggaggactactacccgacgctgtacaagggcgacaccggcctggtcgcccacgagtgcatcctggacctgcgtgaactcacgaaggcctccggcatcaccgccgaggacgtatccaagcgcctgatggacttcggcttccacgccccgaccttggccttcccggtcgccggcaccctgatgatggagcccaccgagtcggaggacaaggaagagctggaccgcttcatcgaagcgatgatcaccatccacggggagatccaggaagttatcgacggcaaggtcaccgccgaacagtccgttctgcgccacgcgccgtttaccgcctattctgttgtgcgggacgacttcgaggaagctgtatccggtggtcacttcagccgtgccaaggccgcatacccggtggctagcctgcgacacaccaagtacttcacccccgtgcgtcgtatcgacaacgcctatggcgaccgcaatctcgtgtgcacctgcccgcccctggaagacttcgcaattaacgaggactagggtccgctggagctaagggcgtcacaaagagaagaaggagaacaagacatgaccgaacttaagaagaccgcgctgcacctggtgcacgagaagttgggcgcgcgatttaccgacttcggcggctgggacatgcctctgaagtacagcagtgagctggacgagcaccacgctgtacgcaatgccgtgggcgtattcgacctctcccacatgggtgaggttcgcgtgaccggcccgcaggcagcggagttcctggaccacgcgctgatttcgaagctgtcggcagtgaaggtcggcaaggcgaagtactcgatgatctgcaccgaatccggtggcatcatcgacgacctgatcacctaccgcctgggcgacaacgagttcctgatcgtgccgaacgcgggcaacgtggacaacgtggtctccgcactgcagggccgcaccgagggctttgacgtggaggttaacaacgagtccgatgcgacctccatgatcgccgtacaggggcccaaggccgcgcaggcgatgctggagatcgtggagaacgtcgtggatgcacccgaggcatccggcgcgggcgagaccgttgccgaggctatcgaggggctgggttactacgcggcattcagcggtgttgccgcaggtcagcccgtgctggtggcccgcacaggctataccggcgaggacggtttcgagctgatcgtggctaacgatggtgcggagaccgtgtggaccaaggctatggaccaggctgcgcagctgggtggcctgccgtgtggcctggcctgccgcgacaccctgcgcctggaggctggcatgccgctgtacggcaacgagctatcgctgaagctcaccccggtcgatgctgggctgggcattcttgcggcgacgaagtctaaggactctttcgttggtcgtgacgccatcgtttccgccaaggaaaagggtacccagcaggtacttatcgggctggcgggcgagggtcgccgcgctgcccgtgggggatacgaggtgtttgccggtgacggcgagaaggccatcggtgccgtgacctccggtgcactgtcgccgacgctgggccacccggtggcattggcatacgtcgcgaagtccgcagtgtcctccggcgcggccgctgagggtgcgaccgtggaggtagacatccgcggcaagcgctttgaatacaaggttgtggcgctgccgttctactcccgcgagaagtaacgcaagaagtaacgaaaagggttgtctgcccgctggtgtaaggtttgggcagacaaccacagaagtaaccacacaaggtttcacaagagaggcttaaaacaatgactgcactgccaactgacttcctgtactccgaagagcacgagtgggttaacacctccgctgttgttgagggcgagaccgtgcgcgtgggcattacccacatcgccgctgaggcgctgggtgacatcgtgttcgtcgagctgccggaggttggctccgaggttgaggccggcgaggctttcggcgaggttgagtccaccaagtccgtttccgacatctacgcaccggtttctggcgaggttgtggctgtcaacgaggcgctggaagacaacgctggcctgatcaacgaagatccatacggtgagggctggctgtacgaggtcaaggtgaccgaggccggcgagctgatggaggctgaggcttaccaggcggctaacgagtaaaacaagaccctatggtcggttccatgttcgcattaatcctcggcttggccctgttgggccttgccgtgtggttactcctgcgagcaaaagatagcaccggatccgccctcccgcacgctttgcgggagggcttttcttttcccggtatttcagtagctcgggggcgtctcattcagcaaacctagctccatatccgcctctgggctctcccaccccgcaccaaaatctctcgcgagaggccttcagggcccttaaatgtcggaaaagttgcaatttgttaatgctactggttttcaaatatgcaaatgcgcaggtcaaggggcttttgggaaagtggaaaccaatcccgatttgaagaattgcaacttttccgacattaagcggccttcaggaagtgccaaacaccggaggcaccagcatcaacgtcaccacctggcatatctagcaccatcagaagcttcagcctcactacaggcggaacctgacgccagcgggacctcacccagcgggacagaacccagcggaacttggcgccagcggaaccgcagccaccagctcccaccagcacccagcagcgccaaggcaccactggagtatcatcgccaccatgggattccagcaaggcagcatccgcaaagcaaacatgggcacaaccggcactaacgacggggcaaccaccccaccagccaacaccagcaccccagccgtagacatcgacgtccgcgacctgggcaccgtggactacgaggacacttggcacctccaggcaaatctcgccgcccagcgcgccgaagaaaaaatcccagacaccatcctcctactccagcatccgccgacgtacaccgccggcaagcgcacccaggattccgaccgccctaccaacggcctgccagtagtcgacgtcgaccgcggaggccgcatcacctggcatggacccggccagttggtcgcatatcccatcatcaagctggccgacccggtggacgtggtcgattatgtccgccgcctggagcaggcgctaatccagacttgtgaagatcttggcctgcacggcaccggccgcgtagaggggcgttcgggcgtgtggctgcctgcgggcgtcattaatggcgagctcaagcccgcacgtaagatagccgcgatcggcatccgcgtgacgcgcggcgtgaccatgcacggagtggccctcaactgcgataacaccatggagtattacgaccacattgtgccttgtgggctggcggatgcgggtgtcacgacgctcaccgaggagctggggcgcgatgttagtgtttctgacgcctactcatccctcgcccacaacctcgttgatgctttgaacggcgacttgccggtgcattcctaggggaaaacccgaggtggattcctatacggcacccagcgtaggctagtcggcatgagtgtgaccgcagatggacgccggatgctacgcatcgaggcgaagaatgccgaaacccccatcgagtcgaagcctcggtggatccgcacgaccgcgaaggtcggcccggagtatcgggatattaagaatcgcgtgaagggcgctggcctgcacaccgtgtgccaagaggctggctgcccgaatatcaacgagtgctgggaggaccgcgaggcgacgttcctcatcggcggtgatacgtgttcccgccgttgcgacttctgccagattaagtccggccgcccgtccccgctggatatggacgagccacgccgcgtggcggagaatgtccgcgagatgggtctgcgctacgccaccatcaccggcgtgacgcgcgatgacctggatgatgagggcgcgtggctgtatgccgaggttgtgaagaagattcacgagctgaacccgaacacgggtgtggagaatctgacgccagatttttccaaccgcccggagctgctgaaggtcgtcttcgattcccagccggaggtgtttgcccacaacctggagacagttccgcgcatctttaagcgcatccgcccggcctttaagtacgaccgttccctggaggtcattcaggcagctcacgattacggcctggtgacgaagtccaacctgatcctgggcatgggtgagaagaaggaagaggtccgcgcggctatcaaggacctggcagacgccggcaccgacattctgacgattacccagtacctgcgcccgtcttccatgcaccacccgattgagcgttgggtgaagccggaggagttcatggagcactccgacgcagcctacgagctgggcatcaaggctgttatgtccggcccgttggtgcgttcctcttaccgcgccggccgcctgtacgcgcaggccaagcaggcgcgcggcgaggctattccggagaacctgaagcacttggaggagactctcgattccaccacgtcgcaggaggcctctacactgctggagcgctacggcgcttcggaggacacgccggtcactgcgtcgcgccgctagctcggcggggcactttcgccccgcctttattcatgccttattgttgaatccatggcgaaggataaaaaggacgtaagggtcaaggaagagaagaaggcagctaaggcaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaaccgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgac

SEQ ID NO: 38 - DNA, 11872 bp, artificial
  Plasmid pOM627
ggatcggcggccagggccctcatggatgaccatccccggcaccatccttgacaccgcccgcacccaagttctggaacagggaattggccttaatcagcagcagttgatggaggttctcaccttgcctgaagagcaaatcccagacttgatggaattagcccaccaggttcggttgaagtggtgtggggaagaaatcgaggtcgagggcattatttccctcaaaactggcggttgccctgaagattgtcatttctgctcacagtctgggttgtttgaatcgccggtgcgttcggtgtggctggatattccgaatctggttgaagccgctaaacagaccgcaaaaactggcgctaccgaattctgtatcgtcgccgcagtcaaggggcctgatgagaggctcatgacccagctggaggaagcagtcctcgcgattcactctgaagttgaaattgaagtcgcagcatcgatcggaacgttaaataaggaacaggtggatcgcctcgctgctgccggcgtgcaccgctagcggccgcttcgcgaagcttgtcgaccgaaacagcagttataaggcatgaagctgtccggtttttgcaaaagtggctgtgactgtaaaaagaaatcgaaaaagaccgttttgtgtgaaaacggtctttttgtttccttttaaccaactgccataactcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaaggaggtgatagtatgtcttctgcagctacccgccgtaattcagcccccttcgttcagcgccacatcggcccaaaccaggccgatacccaggagatcctcgattacctgggctatgaatcttccgccgcgctggccgacgatgccctcccgaagtcgatccgccaggcaggcccgatcggcctgccggaggcactggatgagacggacaccctggccgccctgcgtgcttacgctgacaagaacgtgcagaagcagcagctgatcggcaacggttacttcgacacgatcactccggccgtgattcgccgcaacgtggtggagaacccgggctggtacaccgcctacaccccctaccagccggaaatctcccaggggcgcctcgaggccctgctgaacttccagacgatggtgcaggacctgaccggcctgccagtggccggtgcttcgctgttagacgaagccaccgcagtggccgaggccgtgcagctgatggctcgcggcaatgcgaaggctgccaagaagggcggcgtggtgctgctggattcctccctgcaccagcagtccatcaccgtaaccctggcgcgcgctgaggctgcgggtatcccggtggaggtcgtggacctggacggcgaagatgctaccgccgcgtttgagggccgcgagaacctcgtcggcgtggtgctttccaaccccggctccaccggccgcgtccgcgacctgtccggtctgatctctgcggcgaaggagaccggcgctctggtgacggtcgcttgtgacctgctggctcaggttctggtgacctccccgggctcccaaggtgccgacattgctgtcggctccgcccagcgcttcggcgtgccgctattcttcggtggcccgcacgcgggcttcatctcctgcaccgaggctctgcagcgtaagctgccgggccgcatcgtgggcgtgtccgtggatgccgagggcaccccggcctaccgcttggctctgcagacccgtgagcagcacattcgccgcgacaaggccaccagtaacatctgtaccgctcaggctctgctggccgtcgttgccggtttctacgcggtctggcacggcccagccggcctgcgcgccatcgccgagggggtgcacgcccgcgcgaccgccctggccgttgccctgtccgaggctggcctgacgctggcgcacgataccttcttcgacaccgtcaccgttgacgtgtctggttcttctcttggtgacgcccccacggctctgcgcgccgcagccgaggcaggctacaacctgcgccaggttaacgattccttcgtcggcatctctgtcggcgagtccaccacggacgaggacattgccaagctgatcgaggtgctgggctcccgcaccggcgaggtcaactccgcgagcttcgacgtcaccgccggcccgctgggcgaggccggcgtgctgcgcgcggaggacgaggagattctgacccacccgatcttcaccgccatcacttccgagacccagatgatgcgctacatgcgcaagctggccgaccgcgacctggcgctggatcgtacgatgatcccgctgggctcctgcaccatgaagctgaacgcggccgtctccatggagccgatcacctggcctggcttcgcaggcatccacccgcacgtcccggccgagcaggcgcagggctggctggagctcatcgaggacctggaggagcgcctggcgaagatcaccggctacgccaaggtttccgtccagccgaacgcgggctcccagggcgagttcgccggcctgctggcgatccaccgctaccaccagtcccgcggcgacgatcagcgtgacatcgttctgatcccggcctccgcgcacggcaccaacgctgcctctgcagcgctggcgggcctgaaggtcgtggctgtgaagaatgccgaagacggctccatcgacgtgccggacctggaggccaagctggagaagtacggcgagcagaccgccgccatcatgctgacctacccctccacccacggcgtgttcgaggagcaggtgcgcgacgtctgccagaaggttcacgacgctggcggccaggtgtacgtcgacggcgctaacctgaacgccctggtcggcctggcccagccgggcgagttcggcggcgacgtatcccacctgaacctgcacaagaccttcaccatcccgcacggtggtggcggcccgggcgttggcccggtgtgcgtggcagagcacctgatcccgttcctgcccaccgacccgaacgccgacgttatcgagggcgatgctgccctgcagtccggccagccggtctccggcgcgcagtacggctccgctggcgtgctgccgatcacctggtcctacatcgcacagatgggcgacgaaggcctgactgaggcctcccgcatggccctggtgaacgccaactacgtttcccgcaagctggaggactactacccgacgctgtacaagggcgacaccggcctggtcgcccacgagtgcatcctggacctgcgtgaactcacgaaggcctccggcatcaccgccgaggacgtatccaagcgcctgatggacttcggcttccacgccccgaccttggccttcccggtcgccggcaccctgatgatggagcccaccgagtcggaggacaaggaagagctggaccgcttcatcgaagcgatgatcaccatccacggggagatccaggaagttatcgacggcaaggtcaccgccgaacagtccgttctgcgccacgcgccgtttaccgcctattctgttgtgcgggacgacttcgaggaagctgtatccggtggtcacttcagccgtgccaaggccgcatacccggtggctagcctgcgacacaccaagtacttcacccccgtgcgtcgtatcgacaacgcctatggcgaccgcaatctcgtgtgcacctgcccgcccctggaagacttcgcaattaacgaggactagggtccgctggagctaagggcgtcacaaagagaagaaggagaacaagacatgaccgaacttaagaagaccgcgctgcacctggtgcacgagaagttgggcgcgcgatttaccgacttcggcggctgggacatgcctctgaagtacagcagtgagctggacgagcaccacgctgtacgcaatgccgtgggcgtattcgacctctcccacatgggtgaggttcgcgtgaccggcccgcaggcagcggagttcctggaccacgcgctgatttcgaagctgtcggcagtgaaggtcggcaaggcgaagtactcgatgatctgcaccgaatccggtggcatcatcgacgacctgatcacctaccgcctgggcgacaacgagttcctgatcgtgccgaacgcgggcaacgtggacaacgtggtctccgcactgcagggccgcaccgagggctttgacgtggaggttaacaacgagtccgatgcgacctccatgatcgccgtacaggggcccaaggccgcgcaggcgatgctggagatcgtggagaacgtcgtggatgcacccgaggcatccggcgcgggcgagaccgttgccgaggctatcgaggggctgggttactacgcggcattcagcggtgttgccgcaggtcagcccgtgctggtggcccgcacaggctataccggcgaggacggtttcgagctgatcgtggctaacgatggtgcggagaccgtgtggaccaaggctatggaccaggctgcgcagctgggtggcctgccgtgtggcctggcctgccgcgacaccctgcgcctggaggctggcatgccgctgtacggcaacgagctatcgctgaagctcaccccggtcgatgctgggctgggcattcttgcggcgacgaagtctaaggactctttcgttggtcgtgacgccatcgtttccgccaaggaaaagggtacccagcaggtacttatcgggctggcgggcgagggtcgccgcgctgcccgtgggggatacgaggtgtttgccggtgacggcgagaaggccatcggtgccgtgacctccggtgcactgtcgccgacgctgggccacccggtggcattggcatacgtcgcgaagtccgcagtgtcctccggcgcggccgctgagggtgcgaccgtggaggtagacatccgcggcaagcgctttgaatacaaggttgtggcgctgccgttctactcccgcgagaagtaacgcaagaagtaacgaaaagggttgtctgcccgctggtgtaaggtttgggcagacaaccacagaagtaaccacacaaggtttcacaagagaggcttaaaacaatgactgcactgccaactgacttcctgtactccgaagagcacgagtgggttaacacctccgctgttgttgagggcgagaccgtgcgcgtgggcattacccacatcgccgctgaggcgctgggtgacatcgtgttcgtcgagctgccggaggttggctccgaggttgaggccggcgaggctttcggcgaggttgagtccaccaagtccgtttccgacatctacgcaccggtttctggcgaggttgtggctgtcaacgaggcgctggaagacaacgctggcctgatcaacgaagatccatacggtgagggctggctgtacgaggtcaaggtgaccgaggccggcgagctgatggaggctgaggcttaccaggcggctaacgagtaaaacaagaccctatggtcggttccatgttcgcattaatcctcggcttggccctgttgggccttgccgtgtggttactcctgcgagcaaaagatagcaccggatccgccctcccgcaaagcgtgcgggagggcggtacctgcgcgttcctatttccctgaagttgtcaccactcatacatgggaagagcgccgcgaaactttgcgcctggtggcagaagctggaatggaagtctgttccggcggaatcttaggaatgggcgaaactttagagcagcgcgccgagtttgccgtgcagctggcggagcttgatccgcacgaagtccccatgaacttccttgatcctcgcccgggcaccccatttgccgatagggaattgatggacagccgtgacgctctgcgctctattggtgcgttccgccttgcgatgcctcacaccatgcttcgttttgctggcggtcgcgagctgactttgggcgacaagggttccgagcaagccctcctgggaggcatcaatgcgatgatcgtcggaaactacctgactacgctcggccgcccaatggaagatgacctcgacatgatggatcgtctccagctgcccatcaaagtccttaataaggacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 39 - DNA, 9206 bp, artificial
  Plasmid pOM180
ggatcggcggccagggccctcatgggccgggcgtgcgcaatagactcgtcaccaaaaccgatggagtgtttttgacgctggaagatggcagcaccgtgattgacgcgatgagctcctggtggtcggcaattcatggacacggacacccccgactgaaagctgccgcccaaaaacaaatcgacaccatgagtcacgtcatgtttggcggactaacccacgagcccgccattaagctcacccacaaactcctcaatctcactggaaattcctttgaccacgtcttttattccgattcgggctcggtctcagtggaggtcgccatcaaaatggcactgcaggcctccaaaggacaaggccacccggaacgaacaaaactcctcacctggcggtccggctaccacggagacacattcaccgcgatgagcgtgtgcgacccagaaaatggcatgcatagcctctggaaaggcacactccccgagcagattttcgcccccgccccaccagttcgggggtcatcgccgcaggcgatttccgagtacctgcgcagcatggaattgcttatcgacgagaccgtctccgcaatcatcatcgaaccgatcgtccaaggcgctggaggcatgcgcgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaaggaggtgatagtatgggtttccagcaaggcagcatccgcaaagcaaacatgggcacaaccggcactaacgacggggcaaccaccccaccagccaacaccagcaccccagccgtagacatcgacgtccgcgacctgggcaccgtggactacgaggacacttggcacctccaggcaaatctcgccgcccagcgcgccgaagaaaaaatcccagacaccatcctcctactccagcatccgccgacgtacaccgccggcaagcgcacccaggattccgaccgccctaccaacggcctgccagtagtcgacgtcgaccgcggaggccgcatcacctggcatggacccggccagttggtcgcatatcccatcatcaagctggccgacccggtggacgtggtcgattatgtccgccgcctggagcaggcgctaatccagacttgtgaagatcttggcctgcacggcaccggccgcgtagaggggcgttcgggcgtgtggctgcctgcgggcgtcattaatggcgagctcaagcccgcacgtaagatagccgcgatcggcatccgcgtgacgcgcggcgtgaccatgcacggagtggccctcaactgcgataacaccatggagtattacgaccacattgtgccttgtgggctggcggatgcgggtgtcacgacgctcaccgaggagctggggcgcgatgttagtgtttctgacgcctactcatccctcgcccacaacctcgttgatgctttgaacggcgacttgccggtgcattcctaggggaaaacccgaggtggattcctatacggcacccagcgtaggctagtcggcatgagtgtgaccgcagatggacgccggatgctacgcatcgaggcgaagaatgccgaaacccccatcgagtcgaagcctcggtggatccgcacgaccgcgaaggtcggcccggagtatcgggatattaagaatcgcgtgaagggcgctggcctgcacaccgtgtgccaagaggctggctgcccgaatatcaacgagtgctgggaggaccgcgaggcgacgttcctcatcggcggtgatacgtgttcccgccgttgcgacttctgccagattaagtccggccgcccgtccccgctggatatggacgagccacgccgcgtggcggagaatgtccgcgagatgggtctgcgctacgccaccatcaccggcgtgacgcgcgatgacctggatgatgagggcgcgtggctgtatgccgaggttgtgaagaagattcacgagctgaacccgaacacgggtgtggagaatctgacgccagatttttccaaccgcccggagctgctgaaggtcgtcttcgattcccagccggaggtgtttgcccacaacctggagacagttccgcgcatctttaagcgcatccgcccggcctttaagtacgaccgttccctggaggtcattcaggcagctcacgattacggcctggtgacgaagtccaacctgatcctgggcatgggtgagaagaaggaagaggtccgcgcggctatcaaggacctggcagacgccggcaccgacattctgacgattacccagtacctgcgcccgtcttccatgcaccacccgattgagcgttgggtgaagccggaggagttcatggagcactccgacgcagcctacgagctgggcatcaaggctgttatgtccggcccgttggtgcgttcctcttaccgcgccggccgcctgtacgcgcaggccaagcaggcgcgcggcgaggctattccggagaacctgaagcacttggaggagactctcgattccaccacgtcgcaggaggcctctacactgctggagcgctacggcgcttcggaggacacgccggtcactgcgtcgcgccgctagctcggcggggcactttcgccccgcctttattcatgccttattgttgaatccatggcgaaggataaaaaggacgtaagggtcaaggaagagaagtgatccgccctcccgcacgctttgcgggagggcttttcttttaccggtaccggaactggggttgggaaaaccttctccacagccgttttggttcgatacttagccgatcaaggacacgatgttctgcccgtaaagctagtccaaaccggtgaacttccaggcgagggagacatctttaacattgaacgcttgactggaattgctggagaggaatttgctcgtttcaaagaccctcttgcgccaaatctggcagcccgacgagagggggtcgagccaatacagtttgatcagattatctcgtggcttcgtggttttgacgacccagatcgcatcattgtggtggagggcgctggtggcctgctggtcagattaggggaagatttcaccctggcagatgttgcctccgctttgaatgcacccttagtgattgtgacaagcaccggattgggaagcctcaacgctgctgaattaagcgttgaggcagcaaaccgccgaggactcacagtgttgggagtcctcggcggttcgatccctcaaaatcctgatctagctacgatgcttaatctcgaagaatttgagagagtcaccggcgtgcccttttggggagctttgccggaagggttgtcacgggtggaggggttcgtcgaaaagcaatcttttccggcccttgatgcctttaagaaaccgccggcaaggctcccaacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 40 - DNA, 10003 bp, artificial
  Plasmid pOM331
ggatcggcggccagggccctcatgagatgagtctttgatcaagggtggcgcttttgactcccttggacacgcacgaaaaggcctcatgctggtcttcgaagatgccgttgattccgtcatcgctaccaaaaaagctgctgacaagggacaatttgatctctttgcagctttcgactcggataacaacgacgatgtggcaagtttcttccagatcaccgttcctgatgacgaatgggaccgtaagcatgagctcgcactcgagcgagaaatgctgggtctgtatgtttctggacacccactcgatggctatgaagatgccattgctgcccaggttgatacagcactgaccaccattgttgccggtgaactcaagcacggcgcagaagtgaccgtgggtggcattatctctggtgtggatcgacggttctccaagaaggacggttccccttgggcgattgtcaccattgaagatcacaacggcgcgtccgttgaattgttggtcttcaacaaggtgtattccatcgttggatccatgattgtggaagacaacatcatcttggccaaggcacacatctccattcgagatgatcgtatgagccttttctgtgatgatctccgcgttccagagcttgggccaggaaacgggcaaggacttccgcttcgtttgtccatgcgtactgatcagtgcaccatgtccaacattgccaagctcaagcaggtgctggtggacaacaagggtgaatctgatgtgtacctcaatttgatcgatggggataactccacggtcatgattttgggtgatcacttaagagtcaaccgatccgcaagtttgatgggcgacctcaaggcaacgatggggccaggcatcctcggttaatcacatcacactgggattaccccgtgtaggggtgaaaacccgaatgatgaataaaattccgggtgcagtgaccgtaggtgaggtaaacgcggttagagtcgaatgagagtttgatactttctttcgacttttagattggattttcaatgagccagaaccgcatcaggaccactcacgttggttccttgccccgtaccccagagctacttgatgcaaacatcaagcgttctaacggtgagattggggaggaggaattcttccagattctgcagtcttctgtagatgacctgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaaggaggagaaaacatgtccactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaatttttcgtttgccggaacatccggcaattaaaaaagcggctaaccacgccgctttttttacgtctgcaatttacctttccagtcttcttgctccacggatccgccctcccgcacgctttgcgggagggcggtaccaggtcactgacatcccattcggtgacatcattggtgagatcctgcgcgcagaggtcggtggcttctccttcgaaggcgcatctcctcgtcacgcacacgagtggcgtgtatgggaagaaaacaagcttcctgaaggctctgttatctaccctggtgttgtgtctcactccatcaacgctgtggagcacccacgcctggttgctgatcgtatcgttcagttcgccaagcttgttggccctgagaacgtcattgcgtccactgactgtggtctgggcggacgtctgcattcccagatcgcatgggcaaagctggagtccctagtagagggcgctcgcattgcatcaaaggaactgttctaagctagacaacgagggttgctagtctaagcagcaaaatgagcggctgttgttccttcaggaaaattatctgaaggaacaatagccgctcattttatgtcagtgtgcttttaagcgtcgacgttgatgccaaactgggtgagcatgtcacgcagagtctgcttgaacgatgggtcgacggtcaccatgaaggatgctgcggtggaagcaagagtggtgattgcgagcaatccctgcagtaccgcaaaaggaatggcaagccattctggtggggagacaatggaaccaatcactggcatttcggacaaccgatcgatcagtgcttgctgctcttcgctgatttcatcgtcatcatcgctggtgtctttgccggaggagctcaagcttggggccgcaggaatgtcgccattgctgagcattgagctgccttcagagctgcctggccaggtttcgtttccatcgactggatttccatcatcatcaaggatctgtgatgaggtgatgttgtctgagagctgtgtcagtgcgtcagaggactgagcctgggcaactggagtgaacacggacaatgccacagcgcttgctgtaacaagggtcaaagtacttcgacgcaaagacaaaacttttctcctggcaataaatatgcggatttactatggaaacaagatagaagattggatagcgaaagctatcctcaactcgtggaaagtgtagtgccacaaccacagtattggctagaaaacaatctatagcattgttctacaaagagcttgttggaaataaaacctatgccaaagtaggtgcaattctaggagaagattacactagtcaaccatgagtgaaacatacgtgtctgagaaaagtccaggagtgatggctagcggagcggagctgattcgtgccgccgacattcaaacggcgcaggcacgaatttcctccgtcattgcaccaactccattgcagtattgccctcgtctttctgagatcgagctcacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 41 - DNA, 11325 bp, artificial
  Plasmid pOM344
ttgatcagcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaggaggtgaaagtatggcacagcagacccctttgtacgaacaacacacgctttgcggcgctcgcatggtggatttccacggctggatgatgccgctgcattacggttcgcaaatcgacgaacatcatgcggtacgtaccgatgccggaatgtttgatgtgtcacatatgaccatcgtcgatcttcgcggcagccgcacccgggagtttctgcgttatctgctggcgaacgatgtggcgaagctcaccaaaagcggcaaagccctttactcggggatgttgaatgcctctggcggtgtgatagatgacctcatcgtctactactttactgaagatttcttccgcctcgttgttaactccgccacccgcgaaaaagacctctcctggattacccaacacgctgaacctttcggcatcgaaattaccgttcgtgatgacctttccatgattgccgtgcaagggccgaatgcgcaggcaaaagctgccacactgtttaatgacgcccagcgtcaggcggtggaagggatgaaaccgttctttggcgtgcaggcgggcgatctgtttattgccaccactggttataccggtgaagcgggctatgaaattgcgctgcccaatgaaaaagcggccgatttctggcgtgcgctggtggaagcgggtgttaagccatgtggcttgggcgcgcgtgacacgctgcgtctggaagcgggcatgaatctttatggtcaggagatggacgaaaccatctctcctttagccgccaacatgggctggaccatcgcctgggaaccggcagatcgtgactttatcggtcgtgaagccctggaagtgcagcgtgagcatggtacagaaaaactggttggtctggtgatgaccgaaaaaggcgtgctgcgtaatgaactgccggtacgctttaccgatgcgcagggcaaccagcatgaaggcattatcaccagcggtactttctccccgacgctgggttacagcattgcgctggcgcgcgtgccggaaggtattggcgaaacggcgattgtgcaaattcgcaaccgtgaaatgccggttaaagtgacaaaacctgtttttgtgcgtaacggcaaagccgtcgcgtgatttacttttttggagattgattgatgagcaacgtaccagcagaactgaaatacagcaaagaacacgaatggctgcgtaaagaagccgacggcacttacaccgttggtattaccgaacatgctcaggagctgttaggcgatatggtgtttgttgacctgccggaagtgggcgcaacggttagcgcgggcgatgactgcgcggttgccgaatcggtaaaagcggcgtcagacatttatgcgccagtaagcggtgaaatcgtggcggtaaacgacgcactgagcgattccccggaactggtgaacagcgaaccgtatgcaggcggctggatctttaaaatcaaagccagcgatgaaagcgaactggaatcactgctggatgcgaccgcatacgaagcattgttagaagacgagtaacggctttattcctcttctgcgggagaggatcagggtgaggaaaatttatgcctcaccctcactctcttcgtaaggagagaggttcacaattcactgcacgtttcaggaaccatcgctcatgacacagacgttaagccagcttgaaaacagcggcgcttttattgaacgccatatcggaccggacgccgcgcaacagcaagaaatgctgaatgccgttggtgcacaatcgttaaacgcgctgaccggccagattgtgccgaaagatattcaacttgcgacaccaccgcaggttggcgcaccggcgaccgaatacgccgcactggcagaactcaaggctattgccagtcgcaataaacgcttcacgtcttacatcggcatgggttacaccgccgtgcagctaccgccggttatcctgcgtaacatgctggaaaatccgggctggtataccgcgtacactccgtatcaacctgaagtctcccagggccgccttgaagcactgctcaacttccagcaggtaacgctggatttgactggactggatatggcctctgcttctcttctggacgaggccaccgctgccgccgaagcaatggcgatggcgaaacgcgtcagcaaactgaaaaatgccaaccgcttcttcgtggcttccgatgtgcatccgcaaacgctggatgtggtccgtactcgtgccgaaacctttggttttgaagtgattgtcgatgacgcgcaaaaagtgctcgaccatcaggacgtcttcggcgtgctgttacagcaggtaggcactaccggtgaaattcacgactacactgcgcttattagcgaactgaaatcacgcaaaattgtggtcagcgttgccgccgatattatggcgctggtgctgttaactgcgccgggtaaacagggcgcggatattgtttttggttcggcgcaacgcttcggcgtgccgatgggctacggtggcccacacgcggcattctttgcggcgaaagatgaatacaaacgctcaatgccgggccgtattatcggtgtatcgaaagatgcagctggcaataccgcgctgcgcatggcgatgcagactcgcgagcaacatatccgccgtgagaaagcgaactccaacatttgtacttcccaggtactgctggcaaacatcgccagcctgtatgccgtttatcacggcccggttggcctgaaacgtatcgctaaccgcattcaccgtctgaccgatatcctggcggcgggcctgcaacaaaaaggtctgaaactgcgccatgcgcactatttcgacaccttgtgtgtggaagtggccgacaaagcgggcgtactgacgcgtgccgaagcggctgaaatcaacctgcgtagcgatattctgaacgcggttgggatcacccttgatgaaacaaccacgcgtgaaaacgtaatgcagcttttcaacgtgctgctgggcgataaccacggcctggacatcgacacgctggacaaagacgtggctcacgacagccgctctatccagcctgcgatgctgcgcgacgacgaaatcctcacccatccggtgtttaatcgctaccacagcgaaaccgaaatgatgcgctatatgcactcgctggagcgtaaagatctggcgctgaatcaggcgatgatcccgctgggttcctgcaccatgaaactgaacgccgccgccgagatgatcccaatcacctggccggaatttgccgaactgcacccgttctgcccgccggagcaggccgaaggttatcagcagatgattgcgcagctggctgactggctggtgaaactgaccggttacgacgccgtttgtatgcagccgaactctggcgcacagggcgaatacgcgggcctgctggcgattcgtcattatcatgaaagccgcaacgaagggcatcgcgatatctgcctgatcccggcttctgcgcacggaactaaccccgcttctgcacatatggcaggaatgcaggtggtggttgtggcgtgtgataaaaacggcaacatcgatctgactgatctgcgcgcgaaagcggaacaggcgggcgataacctctcctgtatcatggtgacttatccttctacccacggcgtgtatgaagaaacgatccgtgaagtgtgtgaagtcgtgcatcagttcggcggtcaggtttaccttgatggcgcgaacatgaacgcccaggttggcatcacctcgccgggctttattggtgcggacgtttcacaccttaacctacataaaactttctgcattccgcacggcggtggtggtccgggtatgggaccgatcggcgtgaaagcgcatttggcaccgtttgtaccgggtcatagcgtggtgcaaatcgaaggcatgttaacccgtcagggcgcggtttctgcggcaccgttcggtagcgcctctatcctgccaatcagctggatgtacatccgcatgatgggcgcagaagggctgaaaaaagcaagccaggtggcaatcctcaacgccaactatattgccagccgcctgcaggatgccttcccggtgctgtataccggtcgcgacggtcgcgtggcgcacgaatgtattctcgatattcgcccgctgaaagaagaaaccggcatcagcgagctggatattgccaagcgcctgatcgactacggtttccacgcgccgacgatgtcgttcccggtggcgggtacgctgatggttgaaccgactgaatctgaaagcaaagtggaactggatcgctttatcgacgcgatgctggctatccgcgcagaaattgaccaggtgaaagccggtgtctggccgctggaagataacccgctggtgaacgcgccgcacattcagagcgaactggtcgccgagtgggcgcatccgtacagccgtgaagttgcggtattcccggcaggtgtggcagacaaatactggccgacagtgaaacgtctggatgatgtttacggcgaccgtaacctgttctgctcctgcgtaccgattagcgaataccagtaattcactgattcgactattttctaaaggcgcttcggcgcctttttagtcagatgacaaagtacaaaagtgctcagacagtcccctcgccggatccgccctcccgcacgctttgcgggagggcttttcttttcccggtatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaaccgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgac

SEQ ID NO: 42 - DNA, 200 bp, artificial
  P15 Promoter
tcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgtt

SEQ ID NO: 43 - DNA, 5666 bp, artificial
  Plasmid pHF96
tcgagaggcctgacgtcgggcccggtaccacgcgtctttcgaccaaaccacactgcccacgaatcctagaaatgggatccgtgggcattcgtgtctggtttaacggccttgaaaccggacaggattgcccaaagtagtcgaatttccagttcgtgggcatgtgtgtttggtcaggtttagtcgggtcgaaaaatgaactcggaatcggtaaaaagtgacctcacagaatcgcttctaagggccgttcaaagtgttgcctatacaaacacatgttctgactgcccgacctcttaaaatggcgttaaatggcgcgaaatggaaaacgcctcgtggcctggatggagcgttagtacgagaccagtacgagaccagacacacgtgacaaaaaatcctgaaaagtggaatcattgtcacgcctgtctggtttagctctggttcgggacgggcgtggaatggaggcagtgcaccgagaccttgacccgcggcccgacaagccaaaagtccccaaaacaaacccaccccgccggagacgtgaataaaattcgcagctcattccatcagcgtaaacgcagctttttgcatggtgagacacctttgggggtaaatctcacagcatgaatctctgggttagatgactttctgggtgggggagggtttagaatgtttctagtcgcacgccaaaacccggcgtggacacgtctgcagccgacgcggtcgtgcctgttgtagacggacattcctagtttttccaggagtaacttgatatcttagagatccatttgcttgaaccgccttcccatctttgaattcattcaaggtggtaaggcggttttcgctcttttaatacagttttaaaggtagatttgggagagaagatttcccttaagaaaggttcttaacaaccatgccgcctgcgacgctgttcaatgttttgacttcagctggacttgaccctcaccagtcaggtgatgccattgttgtcgagtctgcccatttcacattgacgttcacgtgggatgagtggctgcgagctcaagcgacgtgggtgggggagttgagtgcgtcggattatgtgcgttctattgtggcgattaactctgcccatgatgcacgggcaacgccgaagatgatgttggatgccccgactggtctgacaacggtgcttaaggcggataagggtcagttgcaggcgtttgccgtggaggcgctgccgattggcgatggcctcagcgaggctcagttggcggggtttgtggctgccgcgtttgatggcgccatcgacctcactcgtgagtttcatgcactttacccggagcgctcgccgcaggagcgcggcgcaatgctcaacattaagcttgtcgacgcctccccctctcaaacagttacgcccgtgcgagtagctaactggttcatggatcagggggtggaggaagttccttatgatgcagcttctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 44 - DNA, 40 bp, artificial
  Plasmid HS1304
tacgaaaggattttttacccatgactaactcttctgcaac

SEQ ID NO: 45 - DNA, 32 bp, artificial
  HS1305
agcgcgtctagattagaacagccccgagataa

SEQ ID NO: 46 - DNA, 32 bp, artificial
  HS1302
gctagaacgcgtgagctgccaattattccggg

SEQ ID NO: 47 - DNA, 40 bp, artificial
  HS1303
gttgcagaagagttagtcatgggtaaaaaatcctttcgta

SEQ ID NO: 48 - DNA, 6965 bp, artificial
  Plasmid pH655
ctagagttctgtgaaaaacaccgtggggcagtttctgcttcgcggtgttttttatttgtggggcactagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgctcgatttaaatctcgagaggcctgacgtcgggcccggtaccacgcgtgagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgactaactcttctgcaaccagcaatccccaacccagcgacgtcgaaatcgcacaggcccacaccctggagcccatcaccacgatcgcggagcgcgccggcatcccggaggccgctttgattccctacggccgtacaaaggccaagatcgacgttcccgctctgcgggcggagcgtgagggcgtcaataaaaagggcaagctcgtgctcgtgaccgcaatgagcccgacccccgcgggtgaaggcaagtccacggtgctgatcggcttggcggacgcggtgcgcacggcagggcgccagacgatggtggcgatccgcgagccgtcccaggggccagtcatgggcatcaagggcggcgctgctggcggcggttatgcgcagatcgtgccgatggaagacatcaacctgcacttcacaggcgacatgcacgccatcaccgccgcgacgaacactctggcggcgatggtggacaaccacgtgcagcacggcaatgctctgggcatcgacccgcggcgggtgacgtggcgacgctgccttgacgtcaacgaccgctcgctgcgccatgtagtcacaggcctaggcggtcctggtcagggcacgccccgcgagggcggattcgatatcacggcggccagcgagatcatggctatcctctgtctggccaccgacctggaggatctaaagaagcgcatcgggcgcatcgtggtgggccagacctacgaccgtcgaccagtcaccgcgggcgacctgaagtgcgcgggcgctatcaccgccctgctgcgcgacgcgatcaacccgaacctggtgcagaccctgggcggcactcccgccctggtgcacggcgggcctttcgccaacatcgcgcacggctgtaactctctgatcgcaaccaccacggcgctggatctatcagaagttgtgctgaccgaggctggtttcggcagcgacctgggtgcggagaagttcttcgacatcaagtcccgcgctggcgacctcgacgtcgccgcaacggttgtggtggccaccatccgttcgctgaagcacaacggcgattccgtgttgaaggccggtctggccaacctggagcgccacgtcagtaacatccgcaagttcggcgtggaacccgtggtggcgctgaacctgtttagctccgataccgccgcggagcgctctatggtcgccgattggggcgagcagttcggtgtgcgcgtggtggagtgcagcgtgtgggctgagggcggtgccggcgcggcggatctggccactgcggtgctggaggtcgttgatggtgtttctgatgaggacgcctccagctccagccaccagatctatcagcccgtcgatggcgtggaagccaccctgcacacacttgccacggaaatctacggcgcggcggatgtgcagttcggcccgcaggctctgaaggacctggcgtttctgaaggacaacggctgggacaagctgccagtgtgcgtgtcgaagacacagtactccttcagcgatgaccccagcgcgctgggcgcgccgagcggccacaccctgcatgtccgcgagctggtgccacgcatcggcgccggattcgtggtggctctcaccggcgacgtgatgactctgccgggtctgccgaagaaacccgcagccgagcgtattgatgtgaacgcgcagggggttatctcggggctgttctaat

SEQ ID NO: 49 - DNA, 6132 bp, artificial
  Plasmid pH657
cgcgtgagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgactaactcttctgcaaccagcaatccccaacccagcgacgtcgaaatcgcacaggcccactccctggagcccatcaccacgatcgcggagcgcgccggcatcccggaggccgctttgattccctacggccgcacaaaggccaagatcgacgttcccgctctgcgggcggagcgtgagggcgtcaatagaaagggcaagctcgtgctcgtgaccgcaatgagcccgacccccgcgggtgaaggcaagtccacggtgctgatcggcttggcggacgcggtgcgcacggcagggcgccagacgatggtggcgatccgcgagccgtcccaggggccggtcatgggcatcaagggcggcgctgctggcggcggttatgcgcagatcgtgccgatggaagacattaacctgcacttcacaggcgacatgcacgccatcaccgccgcgacgaacactctggcggcgatggtggacaaccacgtgcagcacggcaatgctctgggcatcgacccgcggcgggtgacgtggcggcgctgcctggacgtcaacgaccgctcgctgcgccatgtggtcacaggcctaggcggccctggtcagggcacgccccgcgagggcggattcgatatcacggcggccagcgagatcatggctatcctctgtctggccaccgacctggaagatctaaagaagcgcatcgggcgcatcgtggtgggccaaacttatgatcgcaagccagtcaccgcgggcgacctgaagtgcgcgggcgctatcaccgccctgctgcgcgatgcgatcaacccgaacctagtgcagaccctgggtggcacccccgcactggtgcacggtgggcctttcgccaacatcgcgcacggatgcaactcgctgatcgcaaccaccacggcgctggacctgtcggaggttgtgctgaccgaggccggcttcggcagcgacctgggtgcggagaagttcttcgacatcaagtcccgcgccggtggcctcgacgtcgccgcaacggtcgtggtggccaccatccgctcgctaaagcacaacggcgattccgtactaaaggccggtttggccaacctggagcgccacgtcagcaacattcgcaagttcggcgtggaacccgtggtcgcgctaaacctgtttaactctgacaccgccaccgagcgcgctcaggtcgccgactggggcgagcagttcggcgtccgcgtggtggagtgcaacgtgtgggccgagggcggtgccggcgcgaccgatctggcctctgcggtgctggaggtcgttgatggtgtttctgatgaggacgcccccagctccagccaccagatctatcagccggtcgatggtgtggaagccaccttgcgcacacttgccacggagatctacggcgcggcggacgtacagttcggcccgcaggctctgaaggacctggcgtttctgaaggataacggctgggacaagctgccggtgtgcgtgtcgaagacgcagtactccttcagcgatgaccctagcgcgctgggcgcgccaagcggccacaccctgcacgtccgcgagctggtaccacgtatcggtgccggattcgtggtggcactcaccggcgatgtgatgactctgccgggcctgccgaagaaacccgcggccgagcgcattgatgtgaacgcgcagggggttatctcggggctgttctaatctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtacca

SEQ ID NO: 50 - DNA, 5288 bp, artificial
  Plasmid pH670 int sacB delta deformylase
atcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtaccacgcgtaacgcattcactgcagtcacaattgcttcatcactcacaacagctgtttcgaggatgaatttcagcaccacaggagatggaacagcctccctgattgccacaatttcctgcagcaacctattggcatctccctctttcactaccgcaatatccaaaacaacatctacttcggaagctccggactgtacggcaaggcgcgcttcggcggctttcaccaacgcgggggttttgccgtgcgggaatcctgcgacggtggccactcgaattccagcttcttgggcttttgcagttaggttgaccatgctgttggggacacagatcgtgccgactcccagctcaattgcggaatctataaatgcggccagttcggagttggttacttctggtccgaggagggtgtagtcaaggatttgggccatggtggagcgggaaatcgtcatatccataccctacttagacctgacttagtgtgggaaaatttccagggtagaatgcaacgaggctttttgcttttcgacgcgaccctaaacaaatgagacctacccgagggtaggtctcatttgttttgtgtttagtctgtggtggtttcgcgtagtttttcttgcttaataaacagcaagagaagcagcgcgatgccgatcagtggcatcatcacgtagaacactgggatgagtgcgtcgttgtaggaaccggcgaacgcatcgtggagcgcggttggcaattgattgacgattgccggcgtcaattcgttggagtccagtccgccttgggctgccatggcggcttgttcttctggtgaaagttgtgccatggctgctggcattctttcttccatgagggttcccaagttgccaacgaacatgccaccgaccagcgcggatccgagtgaggaaccgatttgacggaagaagttgttcacagcggttgcggatccgaccaccgcggttggcagggtgttctgaacgatcaggaccagaacctgcatggctagaccgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaact

SEQ ID NO: 51 - DNA, 1689 bp, Corynebacterium jeikeium
atgactaactcttctgcaaccagcaatccccaacccagcgacgtcgaaatcgcacaggcccactccctggagcccatcaccacgatcgcggagcgcgccggcatcccggaggccgctttgattccctacggccgcacaaaggccaagatcgacgttcccgctctgcgggcggagcgtgagggcgtcaatagaaagggcaagctcgtgctcgtgaccgcaatgagcccgacccccgcgggtgaaggcaagtccacggtgctgatcggcttggcggacgcggtgcgcacggcagggcgccagacgatggtggcgatccgcgagccgtcccaggggccggtcatgggcatcaagggcggcgctgctggcggcggttatgcgcagatcgtgccgatggaagacattaacctgcacttcacaggcgacatgcacgccatcaccgccgcgacgaacactctggcggcgatggtggacaaccacgtgcagcacggcaatgctctgggcatcgacccgcggcgggtgacgtggcggcgctgcctggacgtcaacgaccgctcgctgcgccatgtggtcacaggcctaggcggccctggtcagggcacgccccgcgagggcggattcgatatcacggcggccagcgagatcatggctatcctctgtctggccaccgacctggaagatctaaagaagcgcatcgggcgcatcgtggtgggccaaacttatgatcgcaagccagtcaccgcgggcgacctgaagtgcgcgggcgctatcaccgccctgctgcgcgatgcgatcaacccgaacctagtgcagaccctgggtggcacccccgcactggtgcacggtgggcctttcgccaacatcgcgcacggatgcaactcgctgatcgcaaccaccacggcgctggacctgtcggaggttgtgctgaccgaggccggcttcggcagcgacctgggtgcggagaagttcttcgacatcaagtcccgcgccggtggcctcgacgtcgccgcaacggtcgtggtggccaccatccgctcgctaaagcacaacggcgattccgtactaaaggccggtttggccaacctggagcgccacgtcagcaacattcgcaagttcggcgtggaacccgtggtcgcgctaaacctgtttaactctgacaccgccaccgagcgcgctcaggtcgccgactggggcgagcagttcggcgtccgcgtggtggagtgcaacgtgtgggccgagggcggtgccggcgcgaccgatctggcctctgcggtgctggaggtcgttgatggtgtttctgatgaggacgcccccagctccagccaccagatctatcagccggtcgatggtgtggaagccaccttgcgcacacttgccacggagatctacggcgcggcggacgtacagttcggcccgcaggctctgaaggacctggcgtttctgaaggataacggctgggacaagctgccggtgtgcgtgtcgaagacgcagtactccttcagcgatgaccctagcgcgctgggcgcgccaagcggccacaccctgcacgtccgcgagctggtaccacgtatcggtgccggattcgtggtggcactcaccggcgatgtgatgactctgccgggcctgccgaagaaacccgcggccgagcgcattgatgtgaacgcgcagggggttatctcggggctgttctaa

SEQ ID NO: 52 - protein, 562 aa, Corynebacterium jeikeium
MTNSSATSNPQPSDVEIAQAHSLEPITTIAERAGIPEAALIPYGRTKAKIDVPALRAEREGVNRKGKLVLVTAMSPTPAGEGKSTVLIGLADAVRTAGRQTMVAIREPSQGPVMGIKGGAAGGGYAQIVPMEDINLHFTGDMHAITAATNTLAAMVDNHVQHGNALGIDPRRVTWRRCLDVNDRSLRHVVTGLGGPGQGTPREGGFDITAASEIMAILCLATDLEDLKKRIGRIVVGQTYDRKPVTAGDLKCAGAITALLRDAINPNLVQTLGGTPALVHGGPFANIAHGCNSLIATTTALDLSEVVLTEAGFGSDLGAEKFFDIKSRAGGLDVAATVVVATIRSLKHNGDSVLKAGLANLERHVSNIRKFGVEPVVALNLFNSDTATERAQVADWGEQFGVRVVECNVWAEGGAGATDLASAVLEVVDGVSDEDAPSSSHQIYQPVDGVEATLRTLATEIYGAADVQFGPQALKDLAFLKDNGWDKLPVCVSKTQYSFSDDPSALGAPSGHTLHVRELVPRIGAGFVVALTGDVMTLPGLPKKPAAERIDVNAQGVISGLF

(19) United States
(12) Patent Application Publication
(10) Pub. No.: US 2010/0003727 A1
(43) Pub. Date: Jan. 7, 2010

(54) Coryneform Bacteria with Formate-THF-Synthetase and/or Glycine Cleavage Activity

(76) Inventors: Oskar Zelder, Speyer (DE); Hartwig Schröder, Nussloch (DE); Corinna Klopprogge, Mannheim (DE); Andrea Herold, Ketsch (DE); Stefan Haefner, Speyer (DE); Thomas A. Patterson, North Attleboro, MA (US); R. Rogers Yocum, Lexington, MA (US); Janice G. Pero, Woburn, MA (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/519,909

(22) PCT Filed: Feb. 14, 2008
(86) PCT No.: PCT/EP08/51795

§ 371 Date: Aug. 27, 2009

(30) Foreign Application Priority Data
Feb. 19, 2007 (EP) 07102652.0

(51) Int. Cl. C12P 13/12; C12N 1/21

(52) U.S. Cl. 435/113; 435/252.3; 435/252.32

(57) ABSTRACT

The present invention relates to microorganisms, in particular C. glutamicum in which the formation of N⁵,N¹⁰-methylene-THF is increased.

The present invention also relates to the use of such microorganisms for producing methionine.

### FIELD OF THE INVENTION

The present invention relates to microorganisms and methods for producing L-methionine. In particular, the present invention relates to Coryneform bacteria which display formate-THF-synthetase activity and/or a functional glycine cleavage system.

### BACKGROUND OF THE INVENTION

Currently, the worldwide annual production of methionine is about 500,000 tons. Methionine is the first limiting amino acid in livestock of poultry and feed and, due to this, mainly applied as feed supplement.

In contrast to other industrial amino acids, methionine is almost exclusively applied as a racemate of D- and L-methionine which is produced by chemical synthesis. Since animals can metabolise both stereo-isomers of methionine, direct feed of the chemically produced racemic mixture is possible (D'Mello and Lewis, Effect of Nutrition Deficiencies in Animals: Amino Acids, Rechgigl (Ed.), CRC Handbook Series in Nutrition and Food, 441-490, 1978).

However, there is still a great interest in replacing the existing chemical production by a biotechnological process producing exclusively L-methionine. This is due to the fact that at lower levels of supplementation L-methionine is a better source of sulfur amino acids than D-methionine (Katz and Baker (1975) Poult. Sci. 545: 1667-74). Moreover, the chemical process uses rather hazardous chemicals and produces substantial waste streams. All these disadvantages of chemical production could be avoided by an efficient biotechnological process.

Fermentative production of fine chemicals such as amino acids, aromatic compounds, vitamins and cofactors is today typically carried out in microorganisms such as Corynebacterium glutamicum (C. glutamicum), Escherichia coli (E. coli), Saccharomyces cerevisiae (S. cerevisiae), Schizzosaccharomycs pombe (S. pombe), Pichia pastoris (P. pastoris), Aspergillus niger, Bacillus subtilis, Ashbya gossypii, Kluyveromyces lactis, Kluyveromyces marxianus or Gluconobacter oxydans.

Amino acids such as glutamate are thus produced using fermentation methods. For these purposes, certain microorganisms such as Escherichia coli (E. coli) and Corynebacterium glutamicum (C. glutamicum) have proven to be particularly suitable. The production of amino acids by fermentation also has inter alia the advantage that only L-amino acids are produced and that environmentally problematic chemicals such as solvents as they are typically used in chemical synthesis are avoided.

Some attempts in the prior art to produce fine chemicals such as amino acids, lipids, vitamins or carbohydrates in microorganisms such as E. coli and C. glutamicum have tried to achieve this goal by e.g. increasing the expression of genes involved in the biosynthetic pathways of the respective fine chemicals.

Attempts to increase production of e.g. lysine by upregulating the expression of genes being involved in the biosynthetic pathway of lysine production are e.g. described in WO 02/10209, WO 2006008097, WO2005059093 or in Cremer et al. (Appl. Environ. Microbiol, (1991), 57(6), 1746-1752).

However, there remains a strong need to identify further targets in metabolic pathways which can be used to beneficially influence the production of methionine in microorganisms such as C. glutamicum.

### OBJECT AND SUMMARY OF THE INVENTION

It is one object of the present invention to provide methods for production of L-methionine in microorganisms.

It is a further object of the present invention to provide microorganisms which produce L-methionine.

These and further objects of the invention, as they will become apparent from the ensuing descriptions, are attained by the subject-matter of the independent claims.

Further embodiments of the invention are defined by the dependent claims.

According to one aspect of the present invention, a method for producing L-methionine is preferred, in a microorganism is provided which comprises the step of culturing a microorganism that is derived by genetic modification from a starting organism such that said microorganism produces more N⁵,N¹⁰-methylene-tetrahydrofolate (THF) compared to the starting organism.

The method uses a microorganism that is selected from the group comprising microorganisms of the genus Enterobacteria, Corynebacterium, Bacillus and Streptomyces. Use of the species Corynebacterium glutamicum is particularly preferred.

In one of the preferred embodiments of the present invention, the method comprises the step of culturing a microorganism that is derived from a genetic modification from a starting organism such that the amount and/or activity of formate-THF-synthetase is increased compared to the starting organism.

A preferred aspect of this latter embodiment of the invention relates to the cultivation of microorganisms in which additionally the amount and/or activity of formate-THF-deformylase is decreased compared to the starting organism and/or in which the amount and/or activity of N⁵,N¹⁰-methenyl-THF-cyclosynthetase, N⁵,N¹⁰-methenyl-THF-reductase and/or N⁵,N¹⁰-methylene-THF-reductase is increased compared to the starting organism.

One of the other preferred embodiments of the present invention relates to methods in which a microorganism is cultivated that is derived by genetic modification from a starting organism such that the enzymatic activity of a glycine cleavage system (GCS) is increased compared to the starting organism.

This may be achieved by genetic modification of a starting organism such that the amount and/or activity of PLP-dependent glycine decarboxylase (gcvP, P-protein), lipoamide-containing aminomethyl transferase (gcvH, H-protein) and N⁵,N¹⁰-methylene-THF-synthesizing enzyme (gcvT, T-protein) are increased compared to the starting organism. These genetic modifications ensure that accumulated glycine is converted to NH₄⁺, CO₂ and N⁵,N¹⁰-methylene-THF. In a further elaboration of this latter aspect of the invention, the microorganisms are cultivated in the presence of lipoic acid and/or lipoamide.

Alternatively or additionally, the microorganisms may be further genetically modified such that they display an increased amount and/or biological activity of lipoic acid synthase (lipA), lipoyl transferase (lipB), and/or lipoic acid synthetase (lplA).

The cultivated microorganisms may additionally or alternatively be genetically modified to display an increased amount and/or activity of an NAD⁺-dependent, FAD-requiring lipoamide dehydrogenase (lpd).

In a preferred embodiment of the present invention the method allows one to produce L-methionine by cultivating microorganisms which have been genetically modified such that the amount and/or biological activity of formate-THF-synthetase is increased and such that a functional glycine cleavage system has been established. Such microorganisms will typically display an increased amount and/or biological activity of formate-THF-synthetase, gcvH, gcvP and gcvT (gcvHPT). In one aspect of the invention, these latter microorganisms will be cultivated in the presence of lipoic acid and/or lipoamide. Alternatively, or additionally, the microorganisms may be further genetically modified to display an increased amount and/or activity of lipA, lipB and/or lplA. The microorganisms may also display an increased amount and/or biological activity of lpd.

The above-described embodiments of methods in accordance with the invention are preferably undertaken by cultivating microorganisms of the species C. glutamicum. The above described genetic modifications may be introduced into a C. glutamicum wild-type strain. In a preferred embodiments, these genetic alterations are introduced into a C. glutamicum strain that already is considered to be a methionine-producing strain.

The coding sequences for the above-mentioned formate-THF-synthetase, gcvP, gcvT, gcvH, IplA, lipA and lipB are preferably derived from C. jeikeium or E. coli. Sequences of C. jeikeium are particularly to be considered in case that the method is undertaken by cultivating C. glutamicum strains.

In another aspect, the present invention relates to microorganisms which have been derived by genetic modification from a starting microorganism to produce more N⁵, N¹⁰-methylene-THF in comparison to the starting organism. The microorganisms can again be selected from the group comprising the genus Enterobacteria, Coryneform bacteria, Bacillus and Streptomyces with Coryneform bacteria and particularly the species C. glutamicum being preferred.

In one embodiment of this aspect of the invention, the microorganism is derived by genetic modification from a starting organism such that the amount and/or activity of formate-THF-synthetase is increased compared to the starting organism. Further elaborations of this latter embodiment of the invention comprise microorganisms with a decrease in the amount and/or activity of formate-THF-deformylase and/or with an increase in any of the activities of N⁵,N¹⁰-methenyl-THF-cyclosynthetase, N⁵,N¹⁰-methenyl-THF-reductase and/or N⁵,N¹⁰-methylene-THF-reductase.

Another aspect of the invention relates to microorganisms which are derived by genetic modification from a starting organism such that the enzymatic activity of a glycine cleavage system is increased in said organism compared to the starting organism.

To this end, the microorganism may be genetically altered in order to display an increased amount and/or activity of gcvP, gcvT and gcvH. In addition, the microorganism may be genetically further modified to display an increased capacity for uptake of externally provided lipoic acid and/or lipoamide and/or for synthesizing endogenously lipoic acid. To this end, the amount and/or activity of IplA, lipA and/or lipB may be increased in said microorganisms. The amount and/or activity of lpd may also be increased compared to the starting organism.

The microorganism is preferably derived from the species of C. glutamicum. The genetic alterations can be introduced in a wild-type strain of C. glutamicum or in a strain which is already considered to be a methionine-producing strain.

A preferred aspect of the present invention relates to microorganisms in which the amount and/or biological activity of formate-THF-synthetase, gcvP, gcvT and gcvH are increased compared to the starting organism. In further elaborations of this aspect of the invention, the amount and/or activity of IplA, lipA and/or lipB can be increased compared to the starting organism. Alternatively and/or additionally, the amount and/or activity of lpd can be increased.

The microorganism is preferably derived from the species of C. glutamicum. The genetic alterations can be introduced in a wild-type strain of C. glutamicum or in a strain which is already considered to be a methionine-producing strain.

### FIGURE LEGENDS

FIG. 1 shows a sequence comparison of the amino acid sequence of lpd of C. jeiekeum and C. glutamicum.

### DETAILED DESCRIPTION OF THE INVENTION

The present invention relates to a method of producing L-methionine, comprising the step of cultivating a genetically modified microorganism and optionally isolating methionine. The present invention also relates to a genetically modified microorganism which is capable of producing L-methionine.

The invention is based on the finding that an efficient production of L-methionine (also designated as methionine) can be achieved in microorganisms if such organisms have been genetically modified from a starting organism such that the resulting microorganism produces more N⁵,N¹⁰-methylene-tetrahydrofolate (THF) compared to the starting organism.

Before describing in detail exemplary embodiments of the present invention, the following definitions are given.

A gene name or a protein name, for example, but not limited to, formate-THF-synthetase, gcvPTH, lipA, lipB, IplA, lpd and any other gene or protein name contained herein, shall refer to either or both the gene and/or the protein or enzyme encoded by said gene, depending on the context in which the name is used.

The term “about” in the context of the present invention denotes an interval of accuracy that the person skilled in the art will understand to be common for the feature in question. The term typically indicates deviation from the indicated numerical value of +/−10%, and preferably +/−5%.

The term “microorganism” for the purposes of the present invention refers to prokaryotes and lower eukaryotes.

The organisms of the present invention thus comprise microorganisms as they are known in the art to be useful for production of fine chemicals such as amino acids, vitamins, enzyme cofactors etc. They can be selected from the genus of Corynebacterium with a particular focus on Coynebacterium glutamicum, the genus of Enterobacteria with a particular focus on Escherichia coli, the genus of Bacillus, with a particular focus on Bacillus subtilis, the genus of actinobacteria, the genus of cyanobacteria, the genus of proteobacteria, the genus of halobacteria, the genus of methanococci, the genus of mycobacteria, the genus of salmonella, the genus of shigella and the genus of streptomycetaceae. Yeasts such as S. pombe, S. cerevisiae, K. lactis, K. marxianus, Ashbya gosypii, and Pichia pastoris are also understood to be encompassed by the term “microorganism”.

As will be explained in detail by the following description, the present invention is primarily concerned with microorganisms that have been genetically modified in order to display an increased amount and/or activity of certain enzymes.

The terms “genetic modification” and “genetic alteration” as well as their grammatical variations within the meaning of the present invention are intended to mean that a microorganism has been modified by means of gene technology to express an altered amount of one or more proteins which can be naturally present in the respective microorganism, one or more proteins which are not naturally present in the respective microorganism, or one or more proteins with an altered activity in comparison to the proteins of the respective non-modified microorganism. A non-modified microorganism is considered to be a “starting organism”, the genetic alteration of which results in a microorganism in accordance with the present invention.

The term “starting organism” therefore can refer to the wild-type of an organism. In the case of C. glutamicum, this may e.g. be ATCC13032. However, the term “starting organism” for the purposes of the present invention may also refer to an organism which already carries genetic alterations in comparison to the wild-type organism of the respective species, but which is then further genetically modified in order to yield an organism in accordance with the present invention.

In case of C. glutamicum, the starting organism may thus be a wild-type C. glutamicum strain such as ATCC13032. However, the starting organism may preferably also be e.g. a C. glutamicum strain which has already been engineered for production of methionine.

Such a methionine-producing starting organism can e.g. be derived from a wild type Coryneform bacterium and preferably from a wild type C. glutamicum bacterium which contains genetic alterations in at least one of the following genes: ask^{fbr}, hom^{fbr} and metH wherein the genetic alterations lead to overexpression of any of these genes, thereby resulting in increased production of methionine relative to methionine produced in the absence of the genetic alterations. In a preferred embodiment, such a methionine producing starter organism will contain genetic alterations simultaneously in ask^{fbr}, hom^{fbr} and metH thereby resulting in increased production of methionine relative to methionine produced in the absence of the genetic alterations.

In these starting organisms, the endogenous copies of ask and hom are typically changed to feedback resistant alleles which are no longer subject to feedback inhibition by lysine threonine, methionine or by a combination of these amino acids. This can be either done by mutation and selection or by defined genetic replacements of the genes by with mutated alleles which code for proteins with reduced or diminished feedback inhibition. A C. glutamicum strain which includes these genetic alterations is e.g. C. glutamicum DSM17322. The person skilled in the art will be aware that alternative genetic alterations to those being described below for generation of C. glutamicum DSM17322 can be used to also achieve overexpression of ask^{fbr}, hom^{fbr} and metH.

For the purposes of the present invention, ask^{fbr} denotes a feedback resistant aspartate kinase. Hom^{fbr} denotes a feedback resistant homoserine dehydrogenase. MetH denotes a Vitamin B12-dependent methionine synthase.

In another preferred embodiment, a methionine-producing starting organism can be derived from a wild type Coryneform bacterium and preferably from a wild type C. glutamicum bacterium which contains genetic alterations in at least one the following genes: ask^{fbr}, hom^{fbr}, metH, metA (also referred to as metX), metY (also referred to as metZ), and hsk^{mutated}. wherein the genetic alterations lead to overexpression of any of these genes, thereby resulting in increased production of methionine relative to methionine produced in the absence of the genetic alterations. In a preferred embodiment, such a methionine producing starter organism will contain genetic alterations simultaneously in ask^{fbr}, hom^{fbr}, metH, metA (also referred to as metX), metY (also referred to as metZ), and hsk^{mutated} thereby resulting in increased production of methionine relative to methionine produced in the absence of the genetic alterations.

In these starting organisms, the endogenous copies of ask, hom and hsk are typically replaced by ask^{fbr}, hom^{fbr} and hsk^{mutated} as described above for ask^{fbr} and hom^{fbr}. A C. glutamicum strain which includes these genetic alterations is e.g. C. glutamicum M2014. The person skilled in the art will be aware that alternative genetic alterations to those being described below specifically for generation of C. glutamicum M2014 can be used to also achieve overexpression of ask^{fbr}, hom^{fbr}, metH, metA (also referred to as metX), metY (also referred to as metZ), and hsk^{mutated}.

For the purposes of the present invention, metA denotes a homoserine succinyl transferase e.g. from E. coli. MetY denotes a O-Acetylhomoserine sulfhydrylase. Hsk^{mutated} denotes a homoserine kinase which has been mutated to show reduced enzymatic activity. This may be achieved by exchanging threonine with serine or alanine at a position corresponding to T190 of hsk of C. glutamicum ATCC13032 with Genbank accession no. Cgl1184. Alternatively or additionally one may replace the ATG start codon with a TTG start codon. Such mutations lead to a reduction in enzymatic activity of the resulting hsk protein compared the non-mutated hsk gene.

In another preferred embodiment, a methionine-producing starting organism can be derived from a wild type Coryneform bacterium and preferably from a wild type C. glutamicum bacterium which contains genetic alterations in at least one of the following genes: ask^{fbr}, hom^{fbr}, metH, metA (also referred to as metX), metY (also referred to as metZ), hsk^{mutated} and metF wherein the genetic alterations lead to overexpression of any of these genes, in combination with a genetic alterations in one of the following genes: serA wherein the genetic alterations decrease expression of this gene where the combination results in increased methionine production by the microorganism relative to methionine production in absence of the combination.

In these starting organisms, the endogenous copy of ask, hom, hsk is replaced as described above and the endogenous copy of serA is typically functionally disrupted. A C. glutamicum strain which includes these genetic alterations is e.g. C. glutamicum OM264C. The person skilled in the art will be aware that alternative genetic alterations to those being described below specifically for generation of C. glutamicum OM264C can be used to also achieve overexpression of ask^{fbr}, hom^{fbr}, metH, metA (also referred to as metX), metY (also referred to as metZ), hsk^{mutated} and metF and reduced expression of serA.

For the purposes of the present invention, serA denotes 3-phosphoglycerate dehydrogenase (see Table 1)

In another preferred embodiment, a methionine-producing starting organism can be derived from a wild type Coryneform bacterium and preferably from a wild type C. glutamicum bacterium which contains genetic alterations in at least one of the following genes: ask^{fbr}, hom^{fbr}, metH, metA (also referred to as metX), metY (also referred to as metZ), hsk^{mutated} and metF wherein the genetic alterations lead to overexpression of any of these genes, in combination with genetic alterations in at least one of the following genes: mcbR and metQ wherein the genetic alterations decrease expression of any of these genes where the combination results in increased methionine production by the microorganism relative to methionine production in absence of the combination. In a preferred embodiment, such a methionine producing starter organism will contain genetic alterations simultaneously in ask^{fbr}, hom^{fbr}, metH, metA (also referred to as metX), metY (also referred to as metZ), hsk^{mutated} and metF wherein the genetic alterations lead to overexpression of any of these genes, in combination with genetic alterations in mcbR and metQ wherein the genetic alterations decrease expression of any of these genes where the combination results in increased methionine production by the microorganism relative to methionine production in absence of the combination.

In these starting organisms, the endogenous copies of ask, hom and hsk are typically replaced as described above while the endogenous copies of mcbR and metQ are typically functionally disrupted or deleted. A C. glutamicum strain which includes these genetic alterations is e.g. C. glutamicum OM469. The person skilled in the art will be aware that alternative genetic alterations to those being described below specifically for generation of C. glutamicum OM469 can be used to also achieve overexpression of ask^{fbr}, hom^{fbr}, metH, metA (also referred to as metX), metY (also referred to as metZ), hsk^{mutated} and metF and reduced expression of mcbR and metQ.

For the purposes of the present invention, metF denotes a N5,10-methylene-tetrahydrofolate reductase (EC 1.5.1.20). McbR denotes a TetR-type transcriptional regulator of sulfur metabolism (Genbank accession no: AAP45010). MetQ denotes a D-methionine binding lipoprotein which function y in methionine import.

In a further preferred embodiment, a methionine-producing starting organism can be derived from a wild type Coryneform bacterium and preferably from a wild type C. glutamicum bacterium which contains genetic alterations in at least one of the following genes: ask^{fbr}, hom^{fbr}, metH, metA (also referred to as metX), metY (also referred to as metZ), hsk^{mutated}, metF, tkt, tal, zwf and 6pgl wherein the genetic alterations lead to overexpression of any of these genes, in combination with genetic alterations in at least one of the following genes: mcbR, metQ and sda wherein the genetic alterations decrease expression of any of these genes where the combination results in increased methionine production by the microorganism relative to methionine production in absence of the combination. In a preferred embodiment, such a methionine producing starter organism will contain genetic alterations simultaneously in ask^{fbr}, hom^{fbr}, metH, metA (also referred to as metX), metY (also referred to as metZ), hsk^{mutated}, metF, tkt, tal, zwf and 6pgl wherein the genetic alterations lead to overexpression of any of these genes, in combination with genetic alterations in mcbR, metQ and sda wherein the genetic alterations decrease expression of any of these genes where the combination results in increased methionine production by the microorganism relative to methionine production in absence of the combination.

A C. glutamicum strain which includes these genetic alterations is e.g. C. glutamicum GK1259. The person skilled in the art will be aware that alternative genetic alterations to those being described below specifically for generation of C. glutamicum GK1259 can be used to also achieve overexpression of ask^{fbr}, hom^{fbr}, metH, metA (also referred to as metX), metY (also referred to as metz), hsk^{mutated}, metF, tkt, tal, zwf and 6pgl and reduced expression of mcbR, metQ and sda.

For the purposes of the present invention, tkt denotes transketolase, tal denotes transaldolase, zwf denotes glucose-6-phosphate-dehydrogenase, 6pgl denotes 6-phospho-glucono-lactonase and sda denotes serine deaminase (see Table 1). The person skilled in the art understands that for increasing the amount and/or activity of zwf, one will also increase the amount and/or activity of opca which serves as a structural scaffolding protein of zwf. In GK1259, this is achieved by the use of the P_{SOD} promoter which simultaneously increases transcription of the pentose phosphate operon comprising tkt, tal, zwf and 6pgl.

As has been set out above, the genetically modified microorganisms of the present invention are characterized in that the amount of N⁵,N¹⁰-methylene-THF is increased.

Typically, the amount of N⁵,N¹⁰-methylene-THF will be increased in the microorganism in accordance with the present invention compared to the respective starting organism by at least about 2%, at least about 5%, at least about 10%, or at least about 20%. In other preferred embodiments, the amount of N⁵,N¹⁰-methylene-THF will be increased by at least about 30%, by at least about 50% or by at least about 75%. Even more preferred embodiments relate to microorganisms in which the amount of N⁵,N¹⁰-methylene-THF is increased by at least about factor 2, at least about factor 5 or at least about factor 10.

The methods and microorganisms in accordance with the present invention can be used to produce more methionine compared to a situation where the respective starting organism, which has not been genetically modified as outlined below, is cultivated. The microorganisms and methods of the present invention can also be used to increase the efficiency of methionine synthesis.

The term “efficiency of methionine synthesis” describes the carbon yield of methionine. This efficiency is calculated as a percentage of the energy input which entered the system in the form of a carbon substrate. Throughout the invention this value is given in percent values ((mol methionine) (mol carbon substrate (⁻¹×100). The term “increased efficiency of methionine synthesis” thus relates to a comparison between the starting organism and the actual Coryneform bacterium in which the amount and/or activity of at least one of the below mentioned enzymes has been increased.

Preferred carbon sources according to the present invention are sugars such as mono-, di- or polysaccharides. For example, sugars selected from the group comprising glucose, fructose, hanose, galactose, ribose, sorbose, lactose, maltose, sucrose, raffinose, starch or cellulose may serve as particularly preferred carbon sources.

The methods and Coryneform bacteria in accordance with the invention may also be used to produce more methionine compared to the starting organism.

The methods and Coryneform bacteria in accordance with the invention may also be used to produce methionine at a faster rate compared to the starting organism. If, for example, a typical production period is considered, the methods and Coryneform bacteria will allow to produce methionine at a faster rate, i.e. the same amount methionine will be produced at an earlier point in time compared to the starting organism. This particularly applies for the logarithmic growth phase.

Methods and Coryneform bacteria in accordance with the invention allow to produce at least about 3 g methionine/l culture volume if the strain is incubated in shake flask incubations. A titer of at least about 4 g methionine/l culture volume, at least about 5 g methionine/l culture volume or at least about 7 g methionine/l culture volume can be preferred if the strain is incubated in shake flask incubations. A more preferred value amounts to at least about 10 g methionine/l culture volume and even more preferably to at least about 20 g methionine/1cell mass if the strain is incubated in shake flask incubations.

Methods and Coryneform bacteria in accordance with the invention allow to produce at least about 25 g methionine/l culture volume if the strain is incubated in fermentation experiments using a stirred and carbon source fed fermentor. An titer of at least about 30 g methionine/l culture volume, at least about 35 g methionine/1 culture volume or at least about 40 g methionine/l culture volume can be preferred if the strain is incubated in fermentation experiments using a stirred and carbon source fed fermentor. A more preferred value amounts to at least about 50 g methionine/1 culture volume and even more preferably to at least about 60 g methionine/1cell mass if the strain is incubated in fermentation experiments using a stirred and carbon source fed fermentor.

In a preferred embodiment, the methods and microorganisms of the invention allow to increase the efficiency of methionine synthesis and/or the amount of methionine and/or the titer and/or the rate of methionine synthesis in comparison to the starting organism by at least about 2%, at least about 5%, at least about 10% or at least about 20%. In preferred embodiments the efficiency of methionine synthesis and/or the amount of methionine and/or the titer and/or the rated is increased compared to the starting organism by at least about 30%, at least about 40%, or at least about 50%.

Even more preferred is an increase of at least about factor 2, at least about factor 3, at least about factor 5 and at least about factor 10.

The term “metabolite” refers to chemical compounds that are used in the metabolic pathways of organisms as precursors, intermediates and/or end products. Such metabolites may not only serve as chemical building units, but may also exert a regulatory activity on enzymes and their catalytic activity. It is known from the literature that such metabolites may inhibit or stimulate the activity of enzymes (Stryer, Biochemistry (2002) W. H. Freeman & Co., New York, N.Y.).

The term “standard conditions” refers to the cultivation of a microorganism in a standard medium which is not enriched with respect to a particular compound. The temperature, pH and incubation time can vary, as will be described in more detail below.

The standard culture conditions for microorganisms can be taken from the literature, including textbooks such as “Sambrook & Russell, Molecular Cloning—A Laboratory Manual”, Cold Spring Harbor Laboratory Press, 3rd edition (2001).

“Minimal media” are media that contain only the necessities for the growth of wild-type or mutant cells, i.e. inorganic salts, a carbon source and water. In the case of mutant cells, a minimal medium can contain one or more additives of substantially pure chemical compounds to allow growth of mutant cells that are deficient in production of such chemical(s).

In contrast, “enriched media” are designed to fulfill all growth requirements of a specific organism, i.e. in addition to the contents of the minimal media, they contain, e.g. amino acids, growth factors, enzyme co-factors, etc.

The term “increasing the amount” of at least one protein (such as formate-THF-synthetase) compared to a starting organism in the context of the present invention means that a starting micororganism is genetically modified to express a higher amount of e.g. one of the above-mentioned enzymes. It is to be understood that increasing the amount of e.g. one enzyme refers to a situation where the amount of functional enzyme is increased. An enzyme such as formate-THF-synthetase in the context of the present invention is considered to be functional if it is capable of catalysing the respective reaction.

There are various options to increase the amount of a protein in microorganisms such as Coryneform bacteria which are well known to the person skilled in the art. These options include increasing the copy number of the nucleic acid sequences which encode the respective protein, increasing transcription and/or translation of such nucleic acid sequences or combinations thereof. These various options will be discussed in more detail below.

The term “increasing the activity” of at least one protein refers to the situation that at least one mutation is introduced into the respective wild-type sequences of the protein which leads to production of more methionine compared to a situation where the same amount of wild-type protein is expressed. This may achieved by e.g. using enzymes which carry specific mutations that allow for an increased activity of the enzyme. Such mutations may e.g. inactivate the regions of the enzymes that are responsible for feedback inhibition. By mutating these positions by e.g. introducing non-conservative point mutations, the enzyme does not provide for feedback regulation any more and thus the activity of the enzyme is not down-regulated if e.g. more product molecules are produced. Furthermore, the activity of an enzyme can be increased by introducing mutations which increase the catalytic turnover of an enzyme. Such mutations may be either introduced into the endogenous copy of the gene encoding for the respective enzyme, or they may be provided by over-expressing a corresponding mutant from the exogenous nucleic acid sequences encoding such an enzyme. Such mutations may comprise point mutations, deletions or insertions. Point mutations may be conservative (replacement of an amino acid with an amino acid of comparable biochemical and physical-chemical properties) or non-conservative (replacement of an amino acid with another which is not comparable in terms of biochemical and physical-chemical properties). Furthermore, the deletions may comprise only two or three amino acids up to complete domains of the respective protein. To give an example, in case of transketolase of C. glutamicum ATCC13032 (Genbank accession no. Cgl1574), a mutation of alanine at a position corresponding to A293 to R and/or alanine at a position corresponding to A327 to T exchange leads to an enzyme with improved enzymatic activity. The person skilled in the art will be able to develop further or alternative mutations based on the information provided herein.

Thus, the term “increasing the activity” of at least one enzyme refers to the situation where mutations are introduced into the respective wild-type sequence to reduce negative regulatory mechanisms such as feedback-inhibition and/or to increase catalytic turnover of the enzyme.

An increase of the amount and/or activity of a protein such as an enzyme may be achieved by different routes, e.g. by switching off inhibitory regulatory mechanisms at the transcriptional, translational or protein level, and/or by increasing gene expression of a nucleic acid encoding for this protein in comparison with the starting organism, e.g. by inducing the endogenous gene or by introducing nucleic acid sequences coding for the protein.

Of course, the approaches of increasing the amount and/or activity of a protein such as an enzyme can be combined. Thus, it is, for example, possible to replace the endogenous copy of an enzyme of Coryneform bacteria with a mutant that encodes for the feedback-insensitive version thereof. If transcription of this mutated copy is set under the control of the strong promoter, the amount and the activity of the respective enzyme is increased. It is understood that in this case the enzyme must still be capable of catalysing the reaction in which it usually participates.

The nucleic acid sequences encoding for a protein such as an enzyme may be of endogenous or exogenous origin. Thus, one may for example increase the amount of a protein such as an enzyme by either increasing expression of nucleic acid sequences that naturally occur within the respective starting microorganism by e.g. chromosomal integration of additional nucleic acid sequences, or by using a strong promoter in front of the endogenous gene. Alternatively or additionally, one may also increase the amount of a protein such as an enzyme by expressing the nucleic acid sequence encoding for a homolog of this enzyme from another organism. Examples for this latter scenario will be put forward below.

Thus, one can e.g. increase the amount of lpd in C. glutamicum by over-expressing the respective C. glutamicum sequence, either from an autonomously replicating vector or from an additionally inserted chromosomal copy (see below) or one may use the corresponding enzymes from e.g. Bacillus subtilis or E. coli and over-express the enzyme by e.g. use of an autonomously replicable vector.

In some circumstances, it may be preferable to use the endogenous enzymes, as the endogenous coding sequence of e.g. C. glutamicum are already optimized with respect to its codon usage for expression in C. glutamicum.

If, in the context of the following description, it is stated that the amount and/or activity of a protein such as of a specific enzyme should be decreased in comparison to the starting organism, the above definitions apply mutatis mutandis.

Reduction of the amount and/or activity of a protein such as an enzyme may be achieved by partially or completely deleting the nucleic acid sequences encoding the respective protein, by inhibiting transcription by e.g. introducing weak promoters, by inhibiting translation by amending the codon usage accordingly, by introducing mutations into the nucleic acid sequences encoding the respective proteins which render the proteins non-functional and/or combinations thereof.

In the context of the following description, use will be made of the term “functional homolog”. The term “functional homolog” for the purposes of the present invention relates to the fact that a certain enzymatic activity may not only be provided by a specific protein of defined amino acid sequence, but also by proteins of similar sequence from other (un)related organisms.

For example, the activity of formate-THF-synthetase can be established in C. glutamicum by expressing nucleic acid sequences which encode for the formate-THF-synthetase of C. jeikeium (SEQ ID NO. 1: nucleic acid sequence, SEQ ID NO. 2: amino acid sequence, gene bank accession number (NP_{—}939608)) or by functional homologs thereof.

Homologues of a protein from other organisms can be easily identified by the skilled person by homology analysis. This can be done by determining similarity, i.e. percent identity between amino acid or nucleic acid sequences for putative homologs and the sequences for the genes or proteins encoded by them (e.g., nucleic acid sequences for formate-THF-synthetase, gcvH, gcvP, gcvT, lpd, IplA, lipA, lipA).

Percent identity may be determined, for example, by visual inspection or by using algorithm-based homology.

For example, in order to determine percent identity of two amino acid sequences, the algorithm will align the sequences for optimal comparison purposes (e.g., gaps can be introduced in the amino acid sequence of one protein for optimal alignment with the amino acid sequence of another protein). The amino acid residues at corresponding amino acid positions are then compared. When a position in one sequence is occupied by the same amino acid residue as the corresponding position in the other, then the molecules are identical at that position. The percent identity between the two sequences is a function of the number of identical positions shared by the sequences (i.e., % identity=# of identical positions/total # of positions multiplied by 100).

Various computer programs are known in the art for these purposes. For example, percent identity of two nucleic acid or amino acid sequences can be determined by comparing sequence information using the GAP computer program described by Devereux et al. (1984) Nucl. Acids. Res., 12:387 and available from the University of Wisconsin Genetics Computer Group (UWGCG). Percent identity can also be determined by aligning two nucleic acid or amino acid sequences using the Basic Local Alignment Search Tool (BLAST™) program (as described by Tatusova et al. (1999) FEMS Microbiol. Lett., 174:247.

At the filing date of this patent application, a standard software package providing the BLAST program can be found on the BLAST website of the NCBI (http://www.ncbi.nlm.nih.gov/BLAST/). For example, if one uses any of the aforementioned SEQ IDs, one can either perform a nucleic acid sequence- or amino sequence-based BLAST search and identify closely related homologs of the respective enzymes in e.g. E. coli, S. cervisiae, Bacillus subtilis, etc. For example, for nucleic acid sequence alignments using the BLAST™ program, the default settings are as follows: reward for match is 2, penalty for mismatch is −2, open gap and extension gap penalties are 5 and 2 respectively, gap.times.dropoff is 50, expect is 10, word size is 11, and filter is OFF.

Comparable sequence searches and analysis can be performed at the EMBL database (http://www.embl.org) or the Expasy homepage (http://www.expasy.org/). All of the above sequences searches are typically performed with the default parameters as they are pre-installed by the database providers at the filing date of the present application. Homology searches may also routinely be performed using software programmes such as the laser gene software of DNA Star, Inc., Madison, Wis., USA, which uses the CLUSTAL method (Higgins et al. (1989), Comput. Appl. Biosci., 5(2) 151).

The skilled person understands that two proteins will likely perform the same function (e.g. provide the same enzymatic activity) if they share a certain degree of identity as described above. A typical lower limit on the amino acid level is typically at least about 25% identity. On the nucleic acid level, the lower limit is typically at least 50%.

Preferred identity grades for both type of sequences are at least about 50%, at least about 60% or least about 70%. More preferred identity levels are at least about 80%, at least about 90% or at least about 95%. These identity levels are considered to be significant.

As used herein, the terms “homology” and “homologous” are not limited to designate proteins having a theoretical common genetic ancestor, but includes proteins which may be genetically unrelated that have, none the less, evolved to perform similar functions and/or have similar structures. The requirement that the homologues should be functional means that the homologues herein described encompass proteins that have substantially the same activity as the reference protein. For proteins to have functional homology, it is not necessarily required that they have significant identity in their amino acid sequences, but, rather, proteins having functional homology are so defined by having similar or identical activities, e.g., enzymatic activities.

Preferably, an enzyme from another organism than e.g. the host Coryneform bacteria will be considered to be a functional homolog if it shows at least significant similarity, i.e. about 50% sequence identity on the amino acid level, and catalyses the same reaction as its counterpart in the Coryneform bacterium. Functional homologues which provide the same enzymatic activity and share a higher degree of identity such as at least about 60%, at least about 70%, at least about 80% or at least about 90% sequence identity on the amino acid level are further preferred functional homologues.

The person skilled in the art knows that one can also use fragments or mutated versions of the aforementioned enzymes from Coryneform bacteria and of their functional homologues in other organisms as long as these fragments and mutated versions display the same type of functional activity. Typical functionally active fragments will display N-terminal and/or C-terminal deletions while mutated versions typically comprise deletions, insertions or point mutations.

By way of example, a sequence of E. coli will be considered to encode for a functional homolog of C. jeikeium formate-THF-synthetase if it displays the above-mentioned identity levels on the amino acid level to SEQ ID NO. 2 and displays the same enzymatic activity. Examples can be taken from Table 1. One can also use fragments or e.g. point mutants of these sequences as long as the resulting proteins still catalyse the same type of reaction as the full-length enzymes.

Examples of increasing the amount of an enzyme will be shown below for formate-THF-synthetase, gcvTHP, IplA, lipA and lipB. Examples for decreasing the amount of an enzyme will be provided for serine deaminase.

Increasing the Amount and/or Activity of Formate-THF-Synthetase in Microorganisms

One preferred aspect of the present invention relates to microorganisms in which a starting organism is genetically manipulated such that the amount and/or activity of formate-THF-synthetase is increased in the resulting microorganism compared to the starting organism. The present invention also relates to methods of producing methionine in microorganisms comprising the step of cultivating the aforementioned microorganism.

In microorganisms such as E. coli and C. jeikeium sequences for formate-THF-synthetase are known. In such microorganisms, increasing the amount and/or activity of formate-THF-synthetase will require raising the amount and/or activity of this enzyme above the level of the respective starting organism by e.g. over-expressing nucleic acid sequences encoding for this enzymatic activity.

In C. glutamicum, which is the preferred host organism of the present invention, a formate-THF-synthetase is not known. The following passages describe how a formate-THF-synthetase activity can be established in C. glutamicum. The person skilled in the art will nevertheless be aware how the amount and/or activity of a formate-THF-synthetase can be increased in other microorganisms such as C. jeikeium and E. coli.

The present invention thus relates inter alia to a C. glutamicum microorganism in which the amount and/or activity of formate-THF-synthetase is increased and the use of such a microorganism for producing methionine. This can be achieved by e.g. increasing the copy number of nucleic acid sequences encoding for a formate-THF-synthetase, increasing transcription and/or translation of sequences encoding a formate-THF-synthetase or a combination thereof.

For the purposes of the present invention, one may use a formate-THF-synthetase from C. jeikeium. The nucleic acid sequence of this formate-THF-synthetase is depicted in SEQ ID NO. 1, while the amino acid sequence is depicted in SEQ ID NO. 2. The gene bank accession number is YP_{—}250663.1. This sequence is derived from the strain C. jeikeium NCTC K11915 which is also designated as K411. A formate-THF-synthetase can also be obtained from the strain DSMZ 7171. In this case, the nucleic acid sequence is depicted by SEQ ID No: 51 and the amino acid sequence is depicted by SEQ ID No. 52.

One may of course also use functional fragments of a formate-THF-synthetase as represented by SEQ ID Nos. 1 and 2, or functional homologs thereof. Some of the homologs which can be identified by using standard homology searches are depicted in Table 1.

The copy number of nucleic acid sequences encoding formate-THF-synthetase can be increased in a microorganism and preferably in C. glutamicum by e.g. either expressing the sequence from autonomously replicating plasmids or by integrating additional copies of the respective nucleic acid sequences into the genome of the microorganism and preferably of C. glutamicum.

In case of autonomously replicable vectors, these can be stably kept within e.g. a Coryneform bacterium. Typical vectors for expressing polypeptides and enzymes such as formate-THF-synthetase in C. glutamicum include pCliK, pB and pEKO as described in Bott, M. and Eggeling, L., eds. Handbook of Corynebacterium glutamicum. CRC Press LLC, Boca Raton, Fla.; Deb, J. K. et al. (FEMS Microbiol. Lett. (1999), 175(1), 11-20), Kirchner O. et al. (J. Biotechnol. (2003), 104 (1-3), 287-299), WO2006069711 and in WO2007012078.

In another approach for increasing the copy number of nucleic acid sequences encoding a polypeptide in a Coryneform bacterium, one can integrate additional copies of nucleic acid sequences encoding such polypeptides into the chromosome of C. glutamicum. Chromosomal integration can e.g. take place at the locus where the endogenous copy of the respective polypeptide is localized. Additionally and/or alternatively, chromosomal multiplication of polypeptide encoding nucleic acid sequences can take place at other loci in the genome of a Coryneform bacterium.

In case of C. glutamicum, there are various methods known to the person skilled in the art for increasing the gene copy number by chromosomal integration. One such method makes e.g. use of the vector pK19 sacB and has been described in detail in the publication of Schafer A, et al. J. Bacteriol. 1994 176(23): 7309-7319. Other vectors for chromosomal integration of polypeptide-encoding nucleic acid sequences include or pCLIK int sacB as described in WO2005059093 and WO2007011845.

Another preferred approach for increasing the amount and/or activity of formate-THF-synthetase in microorganisms and particularly in C. glutamicum is to increase transcription of the coding sequences by use of a strong promoter.

If the activity of an endogenous formate-THF-synthetase is increased by use of a strong promoter, then the term “strong promoter” means that transcription from the newly introduced promoter is stronger than from the naturally occurring endogenous promoter.

However, in a case where formate-THF-synthetase is expressed in C. glutamicum which does not know this type of enzyme, a promoter can be used which is known to provide strong expression of endogenous genes of C. glutamicum.

Preferred promoters in this context are the promoters P_{SOD} (SEQ ID No. 3), P_{grOES} (SEQ ID No 4), P_{EFTu} (SEQ ID No 5), phage SP01 promoter P₁₅ (SEQ ID No 42), and λP_{R} (SEQ ID No 6), also sometimes referred to as lambdaP_{R}. In C. glutamicum the λP_{R} promoter can be stronger than the P_{SOD} promoter. The P_{SOD} promoter can be stronger than the P_{goES} promoter, and the P_{goES} promoter can be weaker than the P_{EFTu} promoter or the P₁₅ promoter. The P_{EFTu} promoter can be stronger than the P_{SOD} promoter. However the strength of a promoter in any organism is not necessarily an inherent property of the promoter, since promoter strength can vary widely depending on the context in which the promoter is placed by the genetic engineering.

The increase of the amount and/or activity of formate-THF-synthetase in microorganisms and particularly in C. glutamicum will allow the microorganisms to grow on media comprising formate as the carbon source. Furthermore, the use of formate which also occurs as a metabolite during various biosynthetic pathways will also allow to increase the production of N⁵,N¹⁰-methylene-THF. An increased level of N⁵,N¹⁰-methylene-THF will lead to an increased production of methyl-THF and production of methionine.

The present invention therefore also relates to a method which comprises culturing the above-described microorganisms and optionally isolating methionine.

In further elaborations of the above-described embodiment of the present invention in which the amount and/or activity of formate-THF-synthetase is increased in a microorganism and preferably in C. glutamicum, the amount of N⁵,N¹⁰-methylene-THF can be further increased by decreasing the amount and/or activity of formyl-THF-deformylase. A nucleic acid sequence for formyl-THF-deformylase is depicted in SEQ ID NO. 7, the amino acid sequence is depicted in SEQ ID NO. 8. Table 1 provides gene bank accession numbers for this enzymatic activity.

Alternatively or additionally, the amount and/or activity of N⁵,N¹⁰-methenyl-THF-cyclosynthetase, N⁵,N¹⁰-methenyl-THF-reductase and/or N⁵,N¹⁰-methylene-THF-reductase are increased compared to the starting organism. In a preferred embodiment, the amount and/or activity of formate-THF-synthetase is increased, the amount and/or activity of formyl-THF-deformylase is decreased and the amount and/or activity of N⁵N¹⁰-methenyl-THF-cyclosynthetase, N⁵,N¹⁰-methenyl-THF-reductase and N⁵,N¹⁰-methenyl-reductase are increased compared to the starting organism. As all of these aforementioned enzymatic activities are present in microorganisms and also in C. glutamicum (with the exception of formate-THF-synthetase), it can be preferred to use the endogenous nucleic acid sequences for increasing and/or decreasing the amount and/or activity of the respective enzymatic activities in the microorganisms in accordance with the present invention and preferably in C. glutamicum.

Approaches for increasing the amount and/or activity for a protein will be described in detail below. These approaches can, of course, also be applied to formate-THF-synthetase. Approaches for decreasing the amount and/or activity of a protein in a microorganism will be described below. These approaches can, of course, also be applied to the down-regulation of formyl-THF-deformylase.

One can, of course, also use functional homologs of formate-THF-synthetase of C. jeikeium or of the other afore-mentioned enzymes. These functional homologs will display the above-mentioned identity grades to either SEQ ID NO. 1 or SEQ ID NO. 2 and provide the same type of enzymatic activity. Accession numbers of formate-THF-synthetase from organisms other than C. jeikeium are provided in Table 1.

A preferred embodiment relates to C. glutamicum microorganisms which express formate THF-synthetase. The present invention also relates preferably to the use of these C. glutamicum organisms in the production of methionine. These strains may show additionally the above mentioned genetic alterations discussed for formyl-THF-deformylase, N⁵,N¹⁰-methenyl-THF-cyclosynthetase, N⁵,N¹⁰-methenyl-THF-reductase and/or N⁵,N¹⁰-methylene-THF-reductase.

A typical C. glutamicum strain that can be used as a starting organism will be a wild-type strain such as ATCC13032. However, it can be preferred to use a starting organism which has already been genetically modified to ensure increased methionine production. Such an organism may display the characteristics of DSM17323 and thus display an increased amount and/or activity of ask^{fbr}, hom^{fbr} and metH. A preferred starting strain may also have the characteristics of M2014 and display an increased amount and/or activity of ask^{fbr}, hom^{fbr}, metH, metA, metY, and hsk^{mutated}. Other preferred starting organisms may have the characteristics of OM469 and display an increased amount and/or activity of ask^{fbr}, hom^{fbr}, metH, metA, metY, hsk^{mutated} and metF and display a reduced amount and/or activity of mcbR and metQ. Yet other preferred starting organisms may have the characteristics of GK1259 and display an increased amount and/or activity of ask^{fbr}, hom^{fbr}, metH, metA, metY, hsk^{mutated}, tkt (and optionally g6pdh, zwfa and 6pgl) and metF and display a reduced amount and/or activity of mcbR, metQ and sda or of M2616 and display an increased amount and/or activity of ask^{fbr}, hom^{fbr}, metH, metA, metY, hsk^{mutated}, tkt (and optionally g6pdh, zwfa and 6pgl) and metF and display a reduced amount and/or activity of mcbR, metQ and serA.

The inventors further found that production of methionine can be further stimulated if one cultivates the above-described microorganisms which display an increased amount and/or activity of formate-THF-synthetase, in a medium containing increased amounts of formate.

This embodiment of the methods in accordance with the invention, where formate is purposively added to the culture medium, is particularly preferably performed with strains of C. glutamicum that have been genetically modified to display the above activities of formate-THF-synthetase and the other genetic alterations. Again, it will be preferred to increase activity of formate-THF-synthetase by expressing the corresponding sequences of C. jeikeium in C. glutamicum or functional homologs and fragments thereof.

Microorganisms with Increased Amount and/or Activity of the Glycine Cleavage System

The present invention in one aspect relates to microorganisms and preferably C. glutamicum which display an increased enzymatic activity of the glycine cleavage system. The present invention also relates to methods which make use of these microorganisms for the production of methionine by cultivating said microorganisms and optionally isolating methionine.

In some industrial applications, microorganisms such as E. coli or C. glutamicum can produce glycine as by-product. The present invention makes use of this by-product by providing microorganisms that display an increased activity of the glycine cleavage system.

The glycine cleavage system of microorganisms is typically comprised of 4 to 5 subunits.

The first subunit is a PLP-dependent glycine decarboxylase (GcvP, also named simply P-protein). The second subunit is a lipoamide-containing amino methyl transferase (GcvH, also names H-protein). The third subunit is a N⁵,N¹⁰-methylene-THF synthesizing enzyme (GcvT). These three factors are sometimes also designated as gcvPTH. The fourth subunit is a NAD⁺-dependent, FAD-requiring lipoamide dehydrogenase (lpd, also named simply L-protein). The corresponding genes are names gcvP, gcvT, gcvH and lpd, respectively. Examples of this type of GCS are found in E. coli and C. jeikeium. The lpd subunit is typically also shared by at least two other multi-subunit enzymes, namely pyruvate dehydrogenase and α-ketoglutarate dehydrogenase.

If the enzymatic activity of the GCS-system is increased, glycine in excess of that required for cell mass will be preferably metabolised into NH₄⁺, CO₂ and N⁵,N¹⁰-methylene-THF, which can then be used e.g. for increased methionine synthesis. However, some microorganisms such as e.g. C. glutamicum lack a native GCS-system. Nevertheless, such organisms will usually have an lpd gene that encodes the subunit for use in the aforementioned two enzyme systems.

As will be shown below, the native Lpd protein in C. glutamicum is able to function together with a non-native GCS such that only the gcvP, gcvT and gcvH genes need to be installed and expressed to obtain an active GCS function in C. glutamicum. It can however be preferred to also over-express a non-native lpd gene, since this gene may be more capable of specifically and efficiently interacting with the gcvP, gcvT and gcvH factors.

Further, it should be noted that in some organisms the glycine cleavage system is comprised of five subunits. For example, in Bacillus subtilis, the P-subunit is e.g. divided into two polypeptides, sometimes named P1 and P2, which are encoded by two genes, sometimes called gcvP1 and gcvP2.

The present invention, as mentioned above, relates in a preferred embodiment to microorganisms that have been genetically modified to display an increased glycine cleavage system activity. Such an increased glycine cleavage system activity can be attained by increasing the amount and/or activity of the enzymatic activities encoded by gcvP, gcvH and gcvT. It will be addressed below how an increased glycine cleavage system activity can be established in C. glutamicum, as this represents a preferred embodiment of the present invention. Nevertheless, the person skilled in the art will be clearly aware how an increased glycine cleavage system activity may also be attained in other organisms such as E. coli or C. jeikeium.

One aspect of the present invention relates to microorganisms, and particularly C. glutamicum, in which the amount and/or activity of the enzymatic activities encoded by gcvP, gcvH and gcvT (collectively called gcvPHT) is increased by genetic alteration compared to the starting organism.

This can be attained by increasing the amount and/or activity of the respective enzymes of C. jeikeium. The nucleic acid sequence of gcvP is depicted in SEQ ID NO. 9, the amino acid sequence is depicted in SEQ ID NO. 10. The Genbank accession number is CAI36361.1.

The nucleic acid sequence of gcvH of C. jeikeium is depicted in SEQ ID NO. 11. The amino acid sequence is depicted in SEQ ID NO. 12. The Genbank accession number is CAI36363.1.

The nucleic acid sequence of gcvT of C. jeikeium is depicted in SEQ ID NO. 13. The amino acid sequence is depicted in SEQ ID NO. 14. The gene bank accession number is CAI36362.1.

For the purposes of the present invention, it can be preferred to use the above sequences which are derived from C. jeikeium .

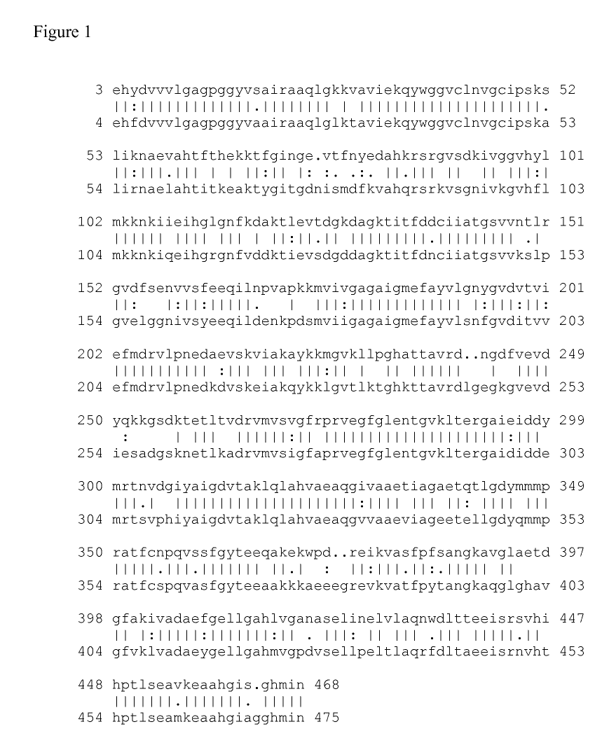

The activity of a glycine cleavage system in a microorganism and particularly in C. glutamicum can be increased by expressing, and preferably over-expressing, the aforementioned sequences, either alone or in combination, with the latter being a particularly preferred embodiment of the present invention. Thus, the present invention particularly relates to C. glutamicum microorganisms in which the enzymatic activities of gcvPHT are concomitantly increased. This may be attained by over-expressing the sequences of gcvP, gcvH and gcvT as depicted by SEQ ID NOS. 9-14, functional fragments thereof, or functional homologs thereof. Functional homologs of the aforementioned sequences of gcvP, gcvH and gcvT can be easily identified through sequence homology searches in the relevant databases and will yield sequences for other organisms such as E. coli. Accession numbers for these enzymes from other organisms are provided in Table 1. The use of gcvP, gcvH and gcvT sequences of C. jeikeium, particularly in C. glutamicum, is preferred, as these genes are clustered in an operon (Tauch et al. (2005) J. Bacteriol., 187, 4671-4682). Moreover, the lpd genes of C. jeikeium and C. glutamicum show a high degree of sequence identity (see FIG. 1). It is reasonable to assume that the gcvP, gcvH and gcvT factors smoothly and efficiently interact with the lpd of C. glutamicum. The amount and/or activity of gcvP, gcvH and gcvT can be increased in microorganisms and preferably in C. glutamicum by the methods mentioned above in the context of formate-THF-synthetase. Thus, one can construct e.g. a functional unit which comprises the coding sequences of gcvP, gcvH and gcvT and increase the copy number of the nucleic acid sequence comprising this unit by using e.g. autonomously replicating plasmids or plasmids which integrate into the genome of the microorganism and preferably into the genome of C. glutamicum.

Alternatively or additionally, one can install a promoter in front of this operon which ensures strong transcription of the coding sequences for gcvP, gcvH and gcvT. Such a promoter may preferably be selected from the group of the P_{EFTu}, P_{grOES}, P_{SOD}, P₁₅ and λ_{PR} promoter.

In principle, it is not necessary to increase the amount and/or activity of the lpd factor. This factor will typically be present in sufficiently abundant amounts by the host microorganism, which is genetically manipulated in order to increase the amount and/or activity of gcvP, gcvH and gcvT. Nonetheless, in some embodiments of the invention, it can be preferred to also increase the amount and/or activity of lpd. To this end, the endogenous sequences of lpd may be over-expressed by any of the above-described methods which are put forward in some more detail below. Depending on the similarity of the lpd of the starting organism and the lpd of the from which gcvP, gcvH and gcvT factors are taken, it can be preferred to increase the amount and/or activity of lpd by increasing expression of endogenous or exogenous lpd. In this case, one will preferably select the lpd of that organism from which the other factors of the glycine cleavage system are taken.

Thus, one embodiment of the present invention relates to microorganisms which are derived from the starting organism such that the resulting microorganism displays an increased amount and/or activity of the factors gcvP, gcvH and gcvT. The invention relates also to methods which use these microorganisms for production of methionine by cultivating the microorganisms and optionally isolating methionine.

A preferred embodiment relates to C. glutamicum microorganisms which express the gcvP, gcvH and gcvT factors of C. jeikeium as depicted in SEQ ID NOS. 9-14, or functional homologs and functional fragments thereof. The present invention also relates preferably to the use of these C. glutamicum organisms in the production of methionine.

A typical C. glutamicum strain that can be used as a starting organism will be a wild-type strain such as ATCC13032. However, it can be preferred to use a starting organism which has already been genetically modified to ensure increased methionine production. Such an organism may display the characteristics of DSM17323 and thus display an increased amount and/or activity of ask^{fbr}, hom^{fbr} and metH. A preferred starting strain may also have the characteristics of M2014 and display an increased amount and/or activity of ask^{fbr}, hom^{fbr}, metH, metA, metY, and hsk^{mutated}. Other preferred starting organisms may have the characteristics of OM469 and display an increased amount and/or activity of ask^{fbr}, hom^{fbr}, metH, metA, metY, hsk^{mutated} and metF and display a reduced amount and/or activity of mcbR and metQ.

The person skilled in the art is furthermore clearly aware that in all of the aforementioned embodiments which relate to an increase in the cleavage glycine system, the starting microorganism which preferably is one of the aforementioned C. glutamicum strains displays further genetic modifications with respect to enzymes that are involved in the serine biosynthetic pathway.

The term “serine biosynthetic pathway” is art-recognized and describes a series of reactions which take place in a wild-type organism and lead to the biosynthesis of serine. The pathway may vary from organism to organism. The details of an organism-specific pathway can be taken from textbooks and the scientific literature which is listed e.g. on the website http://www.genomejp.

Serine is synthesized from the glycolytic intermediate 3-phosphoglycerate which is first oxidized to phosphohydroxypyruvate by the action of 3-phosphoglycerate dehydrogenase (SerA). In a second step, transamination of phosphohydroxypyruvate catalyzed by phosphoserine aminotransferase (SerC) leads to the formation of phosphoserine, which is subsequently dephosphorylated by phosphoserine phosphatase (SerB) to yield L-serine. L-serine can be converted to pyruvate by the serine dehydratase (sdaA) and to glycine and methylene tetrahydrofolate by serine hydroxymethyltransferase (SHMT or glyA).

For the purposes of the present invention, the starting organism in addition to the above-described genetic modifications which aim to introduce an improved glycine cleavage system and to ensure improved use of lipoic acid and/or lipoamide, the amount and/or activity of enzymes selected from the group consisting of D-3-phosphoglycerate dehydrogenase (SerA), phosphoserine phosphotase (SerB), phosphoserine aminotransferase (SerC) and serine hydroxy methyl transferase (SHMT) are increased. Alternatively or additionally, the amount and/or activity of serine deaminase (SdaA) may be reduced. Examples of sequences for the aforementioned enzymes of the serine biosynthesis pathway can be found in Table 1.

A preferred starting strain for may thus have the characteristics of OM264C and display an increased amount and/or activity of ask^{fbr}, hom^{fbr}, metH, metA, metY, and hsk^{mutated} and display a reduced amount and/or activity of serA. Another preferred starting strain for methionine production may have the characteristics of GK1259 and display an increased amount and/or activity of ask^{fbr}, hom^{fbr}, metH, metA, metY, hsk^{mutated}, metF, tkt, tal, zwf and 6pgl and display a reduced amount and/or activity of mcbR, metQ and sda.

Of course, these microorganisms can be particularly preferably be used for the production of methionine in methods in accordance with the invention.

The present inventors furthermore found that N⁵,N¹⁰-methylene-THF and methionine production can be increased in microorganisms that display an increased activity of the glycine cleavage system as described above, if the microorganisms are (i) provided with external lipoic acid and/or lipoamide and/or (ii) are further genetically modified to produce more lipoic acid and/or lipoamide than the starting organism.

The present invention thus in one aspect relates to methods which make use of the above-described microorganisms that display an increased amount and/or activity of the gcvP, gcvH and gcvT factors of the glycine cleavage system (and optionally of the lpd factor) and which are cultivated in a medium containing increased amounts of lipoic acid and/or lipoamide. For the purposes of this aspect of the present invention, lipoic acid and/or lipoamide may be added to the medium up to a final concentration of at least about 0.1 mg/l, at least about 1 mg/ml and preferably at least about 10 mg/l.

It is known in the art that lipoamide, sometimes called lipoic acid amide, can substitute for lipoic acid for the lipoylation of enzymes in some organisms (Reed, L. J. et al. (1958) J. Biol. Chem. 232, 143-158). As such, feeding of lipoamide can substitute for feeding of lipoic acid in the invention disclosed herein. In other words, lipoamide, which is commercially available from the same supplier as lipoic acid in its various forms (for example, Sigma-Aldrich catalog numbers T 5875, T 5625, T 1395, T 8260) can also be used to stimulate or increase glycine cleavage activity in organisms and methods of the invention disclosed herein. Both the oxidized and the reduced forms of these two compounds can be used, as well as various salt and esters of any of these forms. Thus, the source of the lipoyl group can vary.

This embodiment of the methods in accordance with the invention, where lipoic acid and/or lipoamide is purposively added to the culture medium, is particularly preferably performed with strains of C. glutamicum that have been genetically modified to display the activities of the gcvP, gcvH and gcvT factors of a glycine cleavage system. Again, it will be preferred to increase activity of the glycine cleavage system by expressing the corresponding sequences of C. jeikeium in C. glutamicum or functional homologs and fragments thereof. In a further elaboration of this aspect of the invention, the amount and/or activity of the lpd factor may be increased by using either the endogenous C. glutamicum sequences or exogenous sequences (see Table 1).

Also in the case of C. glutamicum will the final concentration of lipoic acid and/or lipoamide in the medium be at least about 0.1 mg/l, at least about 1 mg/l and preferably at least about 10 mg/l.

The microorganisms in accordance with the present invention that display increased activity in the glycine cleavage system resulting from an increased amount and/or activity of the gcvP, gcvH and gcvT factors can be further genetically modified to display an increased amount of internally synthesized lipoic acid and/or lipoamide.

There are two different pathways for making use of lipoic acid and ligating it to target proteins. In E. coli these two pathways are designated as the IplA dependent and the lipB dependent pathway (Morris et al. (1995) J. Bacteriol., 177, 1-10).

The IplA gene encodes for lipoyl synthetase (LplA protein). This enzyme activates lipoic acid with ATP and subsequently attaches lipoyl-AMP to gcvH.

The lipB dependent pathway comprises two enzymes (Morris et al. (1995) J. Bacteriol., 177, 1-10). LipA encodes for lipoic acid synthase (LipA protein). The lipB gene encodes for lipoyl transferase (LipB protein). LipA converts octanoyl-ACP to lipoyl-ACP. In a second step lipB attaches the lipoyl moiety to the lipoyl domain of gcvH and other lipoylated proteins.

Thus, an increase in the amount and/or activity of IplA allows for better incorporation of externally added lipoic acid, while an increase in the amount, type and/or activity of lipA and/or lipB increases the amount of internally synthesized lipoic acid that becomes transferred to GcvH.

A nucleic acid sequence of IplA of E. coli is depicted in SEQ ID NO. 15. The amino acid sequence is depicted in SEQ ID NO. 16. The gene bank accession number is EG1796.

The coding sequence for lipA of C. jeikeium is depicted in SEQ ID NO. 17. The amino acid sequence is depicted in SEQ ID NO. 18. The gene bank accession number is GeneID:3433570.

The nucleic acid sequence for lipB of C. jeikeium is depicted in SEQ ID NO. 19. The amino acid sequence is depicted in SEQ ID NO. 20. The gene bank accession number is GeneID:3433571.

For the purposes of the present invention, it can be preferred to use the above sequences which are derived from C. jeikeium.

A microorganism in accordance with the present invention which displays an increased amount and/or activity of the glycine cleavage system factors gcvP, gcvH and gcvT can thus be further optimized with respect to N⁵,N¹⁰-methylene-THF and methionine synthesis by increasing the amount and/or activity of IplA. To this end, one may increase the expression of IplA by making use of SEQ ID NOS. 15, or functional homologs and fragments thereof. Such a microorganism will show a better incorporation of externally added lipoic acid and/or lipoamide and may thus particularly be suitable for those methods in accordance with the present invention in which the microorganisms are cultivated in the medium being supplemented with lipoic acid and/or lipoamide.

In another preferred embodiment, the present invention relates to a microorganism which, in addition to the increase in the amount and/or activity of the glycine cleavage system factors gcvP, gcvH and gcvT displays an increased amount and/or activity for lipA, lipB or lipA and lipB. A microorganism that in addition to an increased amount and/or activity of gcvP, gcvH and gcvT displays an increased amount and/or activity of lipA and lipB is particularly preferred. These microorganisms may show a better formation and accommodation of endogenously synthesized lipoic acid and/or lipoamide and will thus contribute to the production of N⁵,N¹⁰-methylene-THF and methionine.

Of course, these microorganisms can also be used in the methods in accordance with the present invention which pertain to the cultivation of genetically modified organisms with increased glycine cleavage system activity in medium supplemented with lipoic acid and/or lipoamide. In a further elaboration of this aspect, one may produce and use microorganisms which in addition to an increased amount and/or activity of gcvP, gcvH and gcvT show an increased amount and/or activity of IplA, lipA and lipB.

A particularly preferred embodiment of the present invention again relates to C. glutamicum microorganisms which by way of genetic modification of a starting C. glutamicum organism display an increased amount and/or activity of the glycine cleavage system factors gcvP, gcvH and gcvT and which display improved accommodation of the externally provided lipoic acid and/or lipoamide and/or improved formation and accommodation of internally produced lipoic acid and/or lipoamide by being genetically modified in order to display an increased amount and/or activity of IplA, lipA and/or lipB. Preferred embodiments of the present invention thus relate to C. glutamicum microorganisms in which the amount and/or activity of gcvP, gcvH and gcvT and IplA is increased compared to the starting organism. In another equally preferred embodiment, the C. glutamicum microorganism displays an increased amount and/or activity of gcvP, gcvH and gcvT and lipA or lipB. Even more preferred is a C. glutamicum microorganism which displays an increased amount and/or activity of gcvP, gcvH and gcvT, lipA and lipB. A C. glutamicum microorganism which displays an increased amount and/or activity of gcvP, gcvH and gcvT, lpl, lipA and lipB is can be particularly preferred.

It is understood by the skilled person that the C. glutamicum starting organism which is used for introducing the above-mentioned genetic modification may be a wild-type strain such as ATCC13032. However, it can be preferred to use a starting organism which has already been genetically modified to ensure increased methionine production. Such an organism may display the characteristics of DSM17323 and thus display an increased amount and/or activity of ask^{fbr}, hom^{fbr} and metH. A preferred starting strain may also have the characteristics of M2014 and display an increased amount and/or activity of ask^{fbr}, hom^{fbr}, metH, metA, metY, and hsk^{mutated}. Other preferred starting organisms may have the characteristics of OM469 and display an increased amount and/or activity of ask^{fbr}, hom^{fbr}, metH, metA, metY, hsk^{mutated} and metF and display a reduced amount and/or activity of mcbR and metQ.

The person skilled in the art is furthermore clearly aware that in all of the aforementioned embodiments which relate to an increase in the cleavage glycine system and the improved uptake, formation and accommodation of lipoic acid and/or lipoamide, the starting microorganism which preferably is one of the aforementioned C. glutamicum strains displays further genetic modifications with respect to enzymes that are involved in the serine biosynthetic pathway as described above.

A preferred starting strain may thus have the characteristics of OM264C and display an increased amount and/or activity of ask^{fbr}, hom^{fbr}, metH, metA, metY, and hsk^{mutated} and display a reduced amount and/or activity of serA Another preferred starting strain may have the characteristics of GK1259 and display an increased amount and/or activity of ask^{fbr}, hom^{fbr}, metH, metA, metY, hsk^{mutated}, metF, tkt, tal, zwf and 6pgl and display a reduced amount and/or activity of mcbR, metQ and sda.

Microorganisms with Increased Amount and/or Activity of Formate-THF-Synthetase and of the Glycine Cleavage System

Another preferred embodiment of the present invention refers to microorganisms which combine the properties of the above-mentioned organisms, i.e. increasing the amount and/or activity of formate-THF-synthetase and an increased glycine cleavage system activity. It is understood that the above-described particularly preferred embodiments are also to be combined for this aspect of the present invention.

Thus, a microorganism in accordance with the invention will be genetically modified such that it will display an increased amount and/or activity of formate-THF-synthetase, gcvP, gcvH and gcvT.

In a further preferred embodiment, the microorganism will be further genetically modified to display an increased amount and/or activity of IplA.

A preferred embodiment of the present invention also relates to a microorganism which displays an increased activity of formate-THF-synthetase, gcvP, gcvH and gcvT and lipA or lipB. Even more preferred are microorganisms which display an increased amount and/or activity of a formate-THF-synthetase, gcvP, gcvH and gcvT, lipA and lipB.

Another preferred embodiment relates to a microorganism that displays an increased amount and/or activity of formate-THF-synthetase, gcvP, gcvH and gcvT, IplA, lipA and lipB.

The microorganisms may of course also display an increased amount and/or activity of lpd.

It is understood that the aforementioned embodiments are preferentially realized in a C. glutamicum microorganism. Such a C. glutamicum strain may be a wild-type strain such as ATCC13032. However, it can be preferred to use a starting organism which has already been genetically modified to ensure increased methionine production. Such an organism may display the characteristics of DSM17323 and thus display an increased amount and/or activity of ask^{fbr}, hom^{fbr} and metH. A preferred starting strain may also have the characteristics of M2014 and display an increased amount and/or activity of ask^{fbr}, hom^{fbr}, metH, metA, metY, and hsk^{mutated}. Other preferred starting organisms may have the characteristics of OM469 and display an increased amount and/or activity of ask^{fbr}, hom^{fbr}, metH, metA, metY, hsk^{mutated} and metF and display a reduced amount and/or activity of mcbR and metQ or of M2616 and display an increased amount and/or activity of ask^{fbr}, hom^{fbr}, metH, metA, metY, hsk^{mutated}, tkt (and optionally g6pdh, zwfa and 6pgl) and metF and display a reduced amount and/or activity of mcbR, metQ and serA.

The person skilled in the art is furthermore clearly aware that in all of the aforementioned embodiments which relate to an increase in the cleavage glycine system and the improved uptake, formation and accommodation of lipoic acid and/or lipoamide, the starting microorganism which preferably is one of the aforementioned C. glutamicum strains displays further genetic modifications with respect to enzymes that are involved in the serine biosynthetic pathway as described above and/or enzymes involved in the metabolisation of formyl such as formyl-THF-deformylase, N⁵,N¹⁰-methenyl-THF-cyclosynthetase, N⁵,N¹⁰-methenyl-THF-reductase and/or N⁵, N¹⁰-methylene-THF-reductase.

A preferred starting strain may thus have the characteristics of OM264C and display an increased amount and/or activity of ask^{fbr}, hom^{fbr}, metH, metA, metY, and hsk^{mutated} and display a reduced amount and/or activity of serA. Another preferred starting strain may have the characteristics of GK1259 and display an increased amount and/or activity of ask^{fbr}, hom^{fbr}, metH, metA, metY, hsk^{mutated}, metF, tkt, tal, zwf and 6pgl and display a reduced amount and/or activity of mcbR, metQ and sda.

For increasing the amount and/or activity of the aforementioned enzymes, one may rely either on the endogenous nucleic acid sequences encoding for these enzymes or one may use exogenous sequences, depending on whether the respective starting microorganism provides the required activity.

In case of a C. glutamicum microorganism it will be preferred to use the coding sequences of C. jeikeium for increasing the amount and/or activity of formate-THF-synthetase, gcvP, gcvH and gcvT, IplA, lipA, lipA and/or lpd. The sequences for these enzymes are depicted in SEQ ID NOS. 1 and 2 and 9 to 20. The person skilled in the art is, of course, aware that one may also use sequences coding for functional homologs or fragments of the aforementioned SEQ ID numbers. Such functional homologs may include sequences from other sources such as E. coli. Table 1 provides an overview of possible sequences by reciting corresponding gene bank accession numbers.

The microorganisms and particularly C. glutamicum microorganisms in accordance with the invention can be used in the production of methionine by culturing them and optionally isolating methionine. As mentioned above, the modified organisms may be cultivated in a medium supplemented with increased amounts of formate and lipoic acid and/or lipoamide.

The following table provides an overview of some of the enzymes which have been discussed above in more detail. The gene bank accession numbers recited refer to the gene bank which can be found at the website http://www.ncbi.nlm.nih.gov/.

| Enzyme | Gene bank accession number | Organism |
|---|---|---|
| formate-THF-synthetase | NP_939608, jk0881 DIP1253 SA1553 SAV1732 | C. jeikeum and |
| | MW1675 SAR1810 SAS1658 | others |
| | SAOUHSC_01845 SAUSA300_1678 SAB1592c | |
| | SACOL1782 LSA0947 SH1190 SP_1229 | |
| | Arth_2901 SPD_1087 spr1109 SE1408 | |
| | SERP1295 SAK_1144 M6_Spy0916 | |
| | SpyM3_0853 MGAS2096_Spy0986 SPs1053 | |
| | M5005_Spy_0927 SPy1213 | |
| | MGAS9429_Spy1030 M28_Spy0899 | |
| | MGAS10270_Spy1041 L159505 EF1725 | |
| | stu0791 MGAS10750_Spy1076 STER_0837 | |
| | spyM18_1165 gbs1089 SMU.1073 str0791 | |
| | LSEI_1460 SAG1055 LBA1562 lp_1779 | |
| | LACR_1007 CHY_2385 LVIS_0834 BCE_2187 | |
| | BCZK1914 BT9727_1938 BALH_1871 BC2101 | |
| | lin1990 | |
| formyl-THF-deformylase | ADD13491 NCg10371 cg10382 cg0457 CE0400 | C. glutamicum |
| | RHA1_ro02096 nfa52050 Pfl_4436 PSEEN1152 | and others |
| | PP_1367 PFL_4786 PA4314 PA14_56060 | |
| | HCH_04985 PSPPH_4024 PSPTO_4314 | |
| | Psyr_4018 Tcr_2051 Csal_2073 Noc_1789 | |
| | Daro_0056 ebA3467 Patl_0486 | |
| N5-formyl-THF- | NCgl0845 cgl0881 cgl003 CE0955 DIP0861 | C. glutamicum |
| cyclosynthetase | SAV3674 SCO3183 SCE87.34 Tfu_0372 | and others |
| | nfa49540 MSMEG_5472 Lxx05900 Mmcs_4291 | |
| | Francci3_4041 ML0181 MAV_1101 | |
| | RHA1_ro05631 FRAAL6398 MAP0923c | |
| | Acel_0164 jk1526 aq_1731 | |
| N5,N10-methenyl-THF- | NCgl2091, NP_601375 NCgl0620 cgl0648 cg0750 | C. glutamicum |
| reductase | CE0659 jk1697 DIP0620 MAV_4323 | and others |
| | MAP3463c MSMEG_1647 Rv3356c Mb3391c | |
| | MT3464 ML0674 RHA1_ro06234 Tfu_2571 | |
| | SCO4824 SAV3442 Mmcs_1204 Acel_0385 | |
| | PPA1743 Lxx18630 Arth_1116 | |
| N5,N10-methyleneTHF- | NCgl2091, NP_601375 | C. glutamicum |
| reductase | | |
| PLP-dependent glycine | GeneID: 3433827, Q8FE66, P0A6T9, EG11810, | C. jeikeum, E. coli |
| decarboxylase (gcvP) | g1789269; | and others |
| lipoamide-containing | GeneID: 3433826, CAA52144.1, P0A6T9, | C. jeikeum, E. coli |
| aminomethyltransferase (gcvH) | EG10371, g1789271 | and others |
| N5;N10-methylene-THF | GeneID: 3433828, EG11442, g1789272 | C. jeikeum, E. coli |
| synthesizing enzyme (gcvT) | | and others |
| lipoamide dehydrogenase (lpd) | GeneID: 3433577, CAA52144, Q8FE66, P0A9P0, | C. jeikeum, E. coli |
| | P0A6T9, EG10545, g1786307 | and others |
| Lip A | GeneID: 343570, EG11306, g1786846 | C. jeikeum, E. coli |
| | | and others |
| Lip B | GeneID: 343571, EG11591, gl786848 | C. jeikeum, E. coli |
| | | and others |
| D-3-phosphoglycerate | NCgl1235, CE1379, DIP1104, jk1291, nfa42210, | C. glutamicum |
| dehydrogenase (serA) | MAP3033c, Mb3020c, MT3074, Rv2996c, | and others |
| | ML1692, Tfu_0614, SAV2730, SCO5515, | |
| | Francci3_3637, Lxx13140, CC3215, Jann_0261, | |
| | CHY_2698, MMP1588, VNG2424G, RSP_1352, | |
| | CYB_1383, AGR_L_2264, Atu3706, ZMO1685, | |
| | t1r0325, NP0272A, Mbur_2385, Moth_0020, | |
| | Adeh_1262, SMc00641, RHE_CH03454, | |
| | rrnAC2696, MJ1018, TTE2613, amb3193, | |
| | AF0813, MK0297, DET0599, CYA_1354, | |
| | Synpcc7942_1501, syc2486_c, Saro_2680, | |
| | ELI_01970, MM1753, cbdb_A580, BR1685, | |
| | MTH970, Mbar_A1431, SPO3355, BruAb1_1670, | |
| | BAB1_1697, BMEI0349, SYNW0533, | |
| | Syncc9605_2150, Ava_3759, MA0592, alr1890, | |
| | Mhun_3063, Syncc9902_0527, RPB_1315, | |
| | glr2139, RPD_3905, Nwi_2968, RPA4308, | |
| | SYN_00123, ABC1843, Nham_1119, STH9, | |
| | bll7401, sll1908, CTC00694, BH1602, GK2247, | |
| | RPC_4106, SH1200, Pcar_3115, Gmet_2378, | |
| | SSP1039, BLi02446, BL00647, OB2626, | |
| | BG10509, Acid345_0115, Dgeo_0710, Pro1436, | |
| | SAR1801, SAB1582, SAV1724, SA1545, | |
| | SERP1288, SE1401, SAS1650, MW1666, | |
| | SAOUHSC_01833, SAUSA300_1670, | |
| | SACOL1773, mll3875, GSU1198, HH0135, | |
| | WS1313, Tmden_0875, PMT1431, DR1291, | |
| | PMT9312_1452, TTC0586, Msp_1145, | |
| | At1g17745, TTHA0952, PMM1354, At4g34200, | |
| | RB6248, PMN2A_0926, CJE0970, Cj0891c, | |
| | Pcar_0417, CMC149C, At3g19480, aq_1905, | |
| | jhp0984, HP0397, PH1387, PAB0514, TK1966, | |
| | C31C9.2, PF1394, Cag_1377, TM1401, | |
| | Afu2g04490, CG6287-PA, rrnAC1762, | |
| | AGR_pAT_578, PF0319, Atu5399, PAB2374, | |
| | OB2286, Adeh_1858, BLi03698, BL03435, | |
| | TK0683, PH0597, Reut_B3530, GK1954, | |
| | ABC0220, MK0320, DSY0969, BP0155, | |
| | Bxe_B1896, BB4474, BPP4001, STH3215, | |
| | OB2844, CAC0015, RPC_3076, rrnAC2056, | |
| | RPC_1162, AGR_pAT_470, Atu5328, PP3376, | |
| | PAE3320, Bd1461, Pfl_2987, Rmet_4234, | |
| | CNA07520, GK2965, MS1743, VV11546, | |
| | LA1911, mll1021, MS0068, lp_0785, lin0070, | |
| | VV2851, ebA6869, RPA2975, Tcr_0627, | |
| | LIC11992, TTE1946, MA1334, LMOf2365_0095, | |
| | Sde_3388, lmo0078, LmjF03.0030, SH0752, | |
| | Rmet_4537, orfl9.5263, VP2593, BCE1535, | |
| | RPD_2906, CPE0054, OB2357, bll7965, | |
| | BAS1325, BA_1955, GBAA1434, BA1434, | |
| | Reut_B4747, PFL_2717, PA2263, YPTB3189, | |
| | YP3611, y3301, YPO0914, GOX0218, ACL032C, | |
| | RSP_3407, VC2481, BT9727_1298, BCZK1299, | |
| | BMEII0813, BTH_I2298, Reut_B4615, ECA3905, | |
| | YPTB3910, YP3988, YPO4078, RPB_2550, | |
| | BruAb2_0769, BRA0453, BAB2_0783, Pfl_2904, | |
| | plu3605, PAE1038, DSY1673, Sden_3097, | |
| | NTHI0596, SERP1888, blr4558, Rfer_1867, | |
| | YER081W, BC1415, Pcar_0629, VF2106, y4096, | |
| | SPCC4G3.01, SE1879, SAR2389, BB4731, | |
| | Psyc_0369, TK0551, SCO3478, Csal_1770, | |
| | XCV1890, Bcep18194_A5027, PM1671, | |
| | SAOUHSC_02577, SAUSA300_2254, | |
| | SACOL2296, SAS2196, MW2224, BLi03415, | |
| | BL02138, Mfla_0724, PSPTO5294, XOO3260, | |
| | XC_1568, XCC2550, SAB2178, SAV2305, | |
| | SA2098, PSPPH_4885, XCV2876, SH2023, | |
| | Adeh_2960, BURPS1710b_2286, BPSL1577, | |
| | Pcryo_0410, NE1688, YPTB1320, YP1303, t2980, | |
| | STY3218, Mbar_A2220, Psyr_4852, HI0465, | |
| | y2896, YPO1288, STM3062, SPA2933, YIL074C, | |
| | SERP0516, Bxe_A1982, XAC2724, SC3003, | |
| | BB1050, Afu5g05500, SSP0606, SG2009, SE0622, | |
| | XCC1825, SBO_2700, PF0370, SBO_3080, | |
| | SSO_3065, S3098, SF2898, UTI89_C3299, c3494, | |
| | ECs3784, Z4251, JW2880, b2913, SRU_0653, | |
| | SAB0796, SAR0892, Bd2892, ACIAD3302, | |
| | Saci_1368, SSP1845, Bcep18194_A4216, | |
| | Psyr_1043, Csal_0273, PPA2251, DVU0339, | |
| | PFL_5911, SDY_3169, DDB0230052, SAS0800, | |
| | MW0812, IL2104, PA4626, XC_2364, | |
| | SAUSA300_0834, SACOL0932, SAV0930, | |
| | SA0791, Bpro_1736, SMc01622, amb0136, | |
| | PSPPH_1099, XOO2143, XAC1844, PAB1008, | |
| | RB6394, LBA0942, MCA1407, PSPTO1215, | |
| | PH0520, TM0327, SAOUHSC_00866, BG12409, | |
| | Reut_A2281, ELI_06720, SMc01943, SDY_4350, | |
| | TTC0431, all8087, GSU1672, Nmul_A0428, | |
| | BTH_I2885, BURPS1710b_1481, BPSL1250, | |
| | Ta0779, DSY4020, BLi03716, BL03603, | |
| | amb0195, RSP_3447, UTI89_C4093, ECs4438, | |
| | Z4978, PSHAa0666, PFL_1001, SBO_3555, | |
| | Rru_A2456, Dde_1681, BTH_I1700, Pfl_5387, | |
| | XF2206, S4182, SF3587, c4372, Reut_C5898, | |
| | CPS_2082, SSO_3835, VNG0104G, TTHA0786, | |
| | Pfl_2771, APE1831, SO0862, PD1255, ST1218, | |
| | Moth_1954, BB1529, Csal_0096, SAV7481, | |
| | Bxe_A1055, PP5155, UTI89_C3212, CG1236-PA, | |
| | SSO0905, SAK_1826, gbs1847, SAG1806, | |
| | blr3173, PA0316, ECA0078, DDB0231445, | |
| | SMa2137, JW5656, b3553, GOX0065, | |
| | BURPS1710b_2926, BPSL2459, BMA0513, | |
| | Rmet_2446, SAOUHSC_00142, | |
| | SAUSA300_0179, SACOL0162, SAS0152, | |
| | SAR0178, MW0151, SAV0177, SA0171, | |
| | BPP2132, RSc1034, PP1261, c3405, Dde_3689, | |
| | CAC0089, SMc02849, mlr7269, PTO0372, | |
| | BR2177, RSc3131, Mb0749c, MT0753, Rv0728c, | |
| | DSY3442, SAB0117, Gmet_2695, Noc_2032, | |
| | SC3578, BruAb1_2150, BAB1_2178, BMEI1952, | |
| | BTH_I1402 | |
| phosphoserine | NCgl2436, cg2779, CE2417, DIP1863, jk0483, | C. glutamicum |
| phospatase (serB) | nfa42930, MAP3090c, ML1727, Mb3068c, | and others |
| | MT3127, Rv3042c, SCO1808, SAV6470, | |
| | Tfu_0136, CT0173, Psyr_0557, PSPTO4957, | |
| | PP4909, Sde_1075, HCH_05403, Plut_1948, | |
| | PSPPH_0550, PA4960, ACIAD3567, Pfl_0506, | |
| | Pcar_2283, BF2389, BF2300, RB8037, Cag_0409, | |
| | PFL_0551, PG0653, BT0832, CMI086C, | |
| | Csal_2542, Acid345_2803, AF2138, Lxx11750, | |
| | Rmet_1368, Psyc_1857, AO090020000345, | |
| | Afu3g06550, SPBC3H7.07c, Pcryo_2146, | |
| | SMU.1269, stu1519, str1519, Reut_A1357, | |
| | PBPRA0635, Mfla_1890, Rru_A0465, ACL130C, | |
| | Daro_1962, VV2674, VP2431, YGR208W, | |
| | Bxe_A2331, VV11730, RSc1640, blr6505, | |
| | VF0509, CMQ250C, IL1876, Nwi_2345, | |
| | Bcep18194_A5077, L0085, Z5989, NMB0981, | |
| | SBO_4451, SSO_4538, S4691, SF4420, ECs5346, | |
| | JW4351, b4388, SDY_4649, UTI89_C5159, | |
| | c5473, SAK_0710, gbs0605, SAG0625, MJ1594, | |
| | ECA0465, BL1792, RPB_3347, NMA1179, | |
| | GOX1085, RSP_1350, Tcr_1620, SC4423, | |
| | orf19.5838, NGO1468, YPTB0586, YP3740, | |
| | y3738, YPO0442, STM4578, SPA4388, t4617, | |
| | STY4925, RPA2029, VC2345, ZMO1137, | |
| | CC2097, PPA2051, ebA6034, BURPS1710b_2322, | |
| | BPSL1543, BMA1313, MTH1626, CV3516, | |
| | CPS_1107, RHE_CH02794, AGR_C_3697, | |
| | Atu2040, RPD_2096, Nmul_A0636, BTH_I2264, | |
| | Msp_1096, BB3819, BPP3368, MK0121, | |
| | SPO3353, BP0863, PM1657, SG0398, Mbur_0935, | |
| | HI1033, NTHI1192, RPC_3257, Nham_2724, | |
| | Noc_2504, mlr1449, NE0439, BR1391, | |
| | BMEI0615, BAB1_1410, BruAb1_1387, plu0551, | |
| | SMc01494, MMP0541, SO1223, Jann_0252, | |
| | Bpro_2720, MS1758, amb3479, PSHAa0661, | |
| | MA4429, MM1107, LIC11775, LA2145, | |
| | Sden_1032, Mbar_A1094, Rfer_1329, MCA1267, | |
| | ELI_05525, Saro_2259, WS2081, SPO2363, | |
| | STM2197, NP0274A, SC2213, SPA0654, t0658, | |
| | STY2431, PG1170, rrnAC2717, DDB0230054, | |
| | CJE0330, Cj0282c, VNG2423G, Tmden_1665, | |
| | HP0652, jhp0597, CMP085C, PAB1207, | |
| | CMT542C, TK0052 | |
| phosphoserine | NCgl0794, cg0948, NCgl0794, CE0903, DIP0784, | C. glutamicum |
| aminotransferase (serC) | jk0425, nfa6550, SAV3883, MAP0823c, ML2136, | and others |
| | Tfu_0246, SCO4366, Mb0908c, MT0907, | |
| | Rv0884c, Francci3_0082, Lxx17890, BL1660, | |
| | PPA0483, Jann_0260, SPO3354, GSU3260, | |
| | ZMO1684, Gmet_3173, Saro_2679, RSP_1351, | |
| | Rru_A1104, Sde_1332, CG11899-PA, CPE0053, | |
| | lp_0204, Pcar_2772, BT1153, DSY4684, | |
| | amb3194, rrnAC3046, mll3876, NP0884A, | |
| | Adeh_2622, BF2072, BF2018, Nham_1118, | |
| | Moth_0019, PG1278, Ava_1171, RHE_CH03455, | |
| | LMOf2365_2816, BT9727_3023, SMc00640, | |
| | Mbur_0514, AGR_L_2260, Atu3707, all1683, | |
| | BCE3285, CC3216, 30.t00047, lmo2825, | |
| | Nwi_2969, BruAb1_1672, BR1687, BAB1_1699, | |
| | SG0990, lin2957, BMEI0347, Tbd_0949, | |
| | NTHI1335, BCZK2969, PSPPH_3666, CAC0014, | |
| | CMT252C, GOX1446, RPC_4107, CV2301, | |
| | BCI_0252, Psyr_3646, AF1417, MM2911, | |
| | BC3249, BH1188, RPA4309, bll7402, | |
| | DDB0230053, BAS3079, BA_3823, RPB_1314, | |
| | HI1167, Nmul_A2190, STH3178, L0083, | |
| | Daro_0984, Pfl_4077, PP1768, HD1382, | |
| | 253.t00001, LSL_0091, ECA2594, PTO0371, | |
| | Mbar_A1294, BH03780, PFL_4313, PSPTO1746, | |
| | GBAA3321, BA3321, Bfl383, BQ02790, | |
| | Mfla_1687, y2784, YPO1389, CNL05470, | |
| | RPD_3906, YPTB1414, MA2304, SRU_2207, | |
| | Daro_1231, RSc0903, ELI_01955, HP0736, | |
| | Rfer_1570, PA3167, plu1619, MJ0959, ST0602, | |
| | BG12673, FTT0560c, MS1573, jhp0673, | |
| | FTL_1018, LIC10315, LA0366, lpp1373, RB6246, | |
| | BLi01082, BL05093, NE0333, F26H9.5, DP1933, | |
| | Noc_0172, HCH_04982, PM0837, | |
| | PMT9312_0035, ebA907, Rmet_0715, | |
| | Reut_A2576, lpg1418, PBPRA2455, VF0899, | |
| | str1529, Adeh_2994, BTH_I1966, lpl1369, | |
| | MM0246, Mbar_A2080, Bcep18194_A4155, | |
| | Csal_2167, DR1350, gbs1621, | |
| | BURPS1710b_2651, BPEN_394, rrnAC1999, | |
| | Mhun_2475, Cj0326, Tmden_0073, BPSL2219, | |
| | Pcryo_1434, YP1204, MTH1601, GK0649, | |
| | Tcr_1192, Psyc_1036, PBPRA3292, stu1529, | |
| | SYN_00124, STM0977, SPA1821, MA1816, | |
| | SC0931, t1957, STY0977, TK1548, CJE0371, | |
| | BMA1625, Dgeo_1114, XOO2388, SSO2597, | |
| | BURPS1710b_2998, BMA0433, VV11425, | |
| | UTI89_C0978, c1045, MK0633, HH0909, | |
| | ACIAD2647, ABC1531, MCA1420, SSO_0908, | |
| | S0966, SF0902, BPSL2519, WGLp486, bbp289, | |
| | SBO_2193, AO090023000099, Bxe_A0976, | |
| | IL1359, SDY_2354, ECs0990, Z1253, JW0890, | |
| | b0907, VV21664, Syncc9605_0044, VP2714, | |
| | VP1247, XC_2645, XCC1589, SMa1495, | |
| | BTH_I1634, PSHAa1422, VC1159, cbdb_A581, | |
| | STH8, VVA0476, SPAC1F12.07, PAB1801, | |
| | WS0024, VV2958, TTHA0582, CT0070, | |
| | PMM0035, NMA1894, VV1451, VV12813, | |
| | VPA0235, BU312, At4g35630, TTC1813, | |
| | RHE_PB00131, NMB1640, CPS_2190, XAC1648, | |
| | TTC0213, Bpro_1793, Sden_0404, XF2326, | |
| | At2g17630, PH1308, MMP0391, DET0600, | |
| | Tbd_2509, VF0339, TTHA0173, NP2578A, | |
| | VCA0604, Saci_0249, NGO1283, VC0392, | |
| | SMU.1656 | |
| serine dehydratase (sdaA) | NCgl1583, _NCgl0939 | C. glutamicum |
| serine | Q93PM7, BA000035, Q8FQR1, Q6NI47, Q4JU69, | C. glutamicum |
| hydroxymethyltransferase | Q5YQ76, Q73WG1, Q4NIE8, O53441, P59953, | and others |
| (shmt) | Q6ADF0, Q9X794, Q40XZ1, Q82JI0, O86565, | |
| | Q47MD6, Q4NM56, ORF, Q4NGB0, BX251412, | |
| | O53615, P66806, Q7U2X3, Q24MM6, Q2ZEP1, | |
| | Q65DW5, Q426V7, Q5KUI2, Q2RFW7, Q3A934, | |
| | Q3CJJ0, Q8R887, Q8Y4B2, Q4EPI3, Q71WN9, | |
| | Q67N41, Q927V4, P39148, Q5HE87, Q2YUJ1, | |
| | Q9K6G4, Q7SIB6, Q2FF15, Q5WB66, Q4CID1, | |
| | Q2BG18, Q40L42, Q3AN03, Q8YMW8, O66776, | |
| | Q1YIN1, Q41G88, Q74CR5, Q3GAC7, Q3MBD8, | |
| | Q2DMQ8, Q7U9J7, Q39V87, Q630T3, Q72XD7, | |
| | Q2D1V8, Q6HAW9, AE017221, Q5SI56, Q72IH2, | |
| | Q814V2, Q5HMB0, Q8XJ32, AE017225, Q81JY4, | |
| | Q3WZQ2, CP000360, Q5NN85, Q3AW18, | |
| | Q4L7Z4, Q3A4L9, Q26LA5, Q7V4U3, Q6FA66, | |
| | Q2S9R4, Q3G5N8, Q2SFI7, Q3N8U1, Q6N693, | |
| | Q82UP9, Q2JT50, Q31CS4, Q5P7P1, Q9HTE9, | |
| | Q3KDV1, Q26XG3, Q3SGX5, Q5FNK4, Q2ILI1, | |
| | S30382, Q2YD58, Q4BPZ9, Q2LQM6, Q5N2P9, | |
| | Q376I5, Q46HB6, P50435, Q37NB6, Q7ND67, | |
| | Q72CT0, Q2WMW5, Q2CH39, Q7VDS8, | |
| | Q8U7Y5, Q88AD1, O85718, Q48CP3, Q2JI36, | |
| | Q8DH33, Q7V335, Q214H7, Q6MLK1, Q3QXZ6, | |
| | Q2DFI0, Q9WZH9, CP000283, Q37FB0, Q3N0F7, | |
| | Q4ZM83, Q44AR5, Q8EM73, Q1WTR3, Q2CP12, | |
| | Q3SRV3, Q3CCS2, Q2W4T2, Q35IU4, Q2IWS4, | |
| | Q2CQJ5, Q4J3C4, Q49Z60, CT573326, Q4C6H0, | |
| | Q31ZN2, Q607U4, P24060, Q4BQS8, Q41LQ8, | |
| | Q7UQN2, Q2YN95, Q2RTB8, Q3P773, Q46RR4, | |
| | Ser, Q47IH1, Q3JGP5, CT573326, Q21NP8, | |
| | Q3F809, Q2T437, Q3F764, Q88R12, S15203, | |
| | Q4K4P6, Q5X722, Q8YGG7, Q3VCK5, | |
| | Q5WYH4, CP000271, Q1UEA8, Q4LV45, | |
| | Q8G1F1, Q9I138, P77962, P34895, Q62DI5, | |
| | Q1QMB9, Q1V9T1, Q2BLZ4, Q30YL7, Q8XTQ1, | |
| | Q92QU6, Q97GV1, Q39A26, Q45D73, | |
| | AM180252, Q3WQZ9, Q9KMP4, Q2KA25, | |
| | Q4LY56, Q2S4G9, Q8D7G5, Q36MR4, Q28N04, | |
| | Q3K5K9, CP000254, BA000038, Q4B4P5, | |
| | Q2FLH5, Q7NYI8, Q7MEH7, Q6N622, Q2RVA2, | |
| | Q3XRF3, Q303B4, Q7N216, Q47WY2, Q4UQT6, | |
| | Q481S6, Q4BM61, Q4BA21, Q3FFQ1, Q3HGC4, | |
| | Q87I03, Q3FB08, Q5LPA8, Q88UT5, Q92XS8, | |
| | Q3QH38, Q34W82, Q39J72, Q8Y1G1, CP000152, | |
| | AM236080, H97501, Q391K1, Q8UG75, Q21V29, | |
| | Q474L3, Q8KC36, Q3APN5, CP000124, Q3BXI8, | |
| | Q62I16, Q831F9, Q1QE01, Q3CX04, Q2NZ83, | |
| | Q8PPE3, CP000352, Q3S0V7, Q2AFR6, Q8TK94, | |
| | CP000086, Q2BI80, Q2G646, Q3J9K8, Q47XG4, | |
| | Q2SYS4, Q1YWG2, Q73GC3, Q44LK7, Q33Y20, | |
| | Q2NS25, Q2CGY4, Q5GTS7, Q36D93, | |
| | AE008384, Q8PZQ0, Q9HVI7, Q983B6, | |
| | CP000270, Q3R0R3, Q2Z5R9, Q1VX33, | |
| | Q4ZNH2, Q4FUZ8, Q72PY2, Q48DU7, Q3VPD3, | |
| | Q6LHN7, AE009442, Q87AS2, Q3B2I7, | |
| | Q87WC1, Q7WFD2, Q4K5R9, Q1R8I4, P0A826, | |
| | CT573326, Q3WKF8, Q2ZQD2, Q8EBN8, | |
| | Q8XA55, Q3ZZG3, Q2J6M3, Q2DUP7, Q9XAZ1, | |
| | Q3K6J0, Q3Q439, Q9A8J6, Q7W400, Q3YZ04, | |
| | Q32D21, Q5F8C0, Q4AMK6, Q6D246, Q3R828, | |
| | E82743, Q9PET2, Q3P6F8, Q9XAY7, Q3NK51, | |
| | Q3Z9B9, Q6G3L3, Q88Q27, Q1ZIE9, Q31FS6, | |
| | P56990, Q9XB01, Q3DHL3, Q6LU17, Q7W1I6, | |
| | H82258, Q9KTG1, Q8E5C6, Q57LF7, Q3IRX5, | |
| | Q3K122, Q7WPH6, AP008231, Q1YU48, | |
| | Q3D8P3, Q8DPZ0, Q97R16, Q46A52, Q6F211, | |
| | Q1PZE1, Q8L372, B48427, Q2KV15, Q1RGX5, | |
| | Q43K52, Q3VUL2, Q3II23, Q1ZPS2, Q2NAR9, | |
| | Q8DU67, Q9CHW7, Q6CZV5, Q3XBK9, H84295, | |
| | Q4FLT4, Q1UZA1, Q8DFC9, Q74LC1, Q488N6, | |
| | Q2C6B3, Q65T08, Q1Z7P1, F75567, Q9HPY5, | |
| | Q9RYB2, Q1V311, Q87RR2, Q3GI80, Q6G009, | |
| | Q8ZCR1, Q5QXT4, Q5V3D7, Q2ST43, Q5E706, | |
| | Q8Z2Z9, Q1XXG3, Q5PBM8, Q6MS85, | |
| | Q3EFW1, Q7QM11, Q2BUE3, Q48TK6, | |
| | Q5FMC0, BA000034, Q1U7W2, Q8P122, | |
| | Q8K7H8, Q99ZP1, Q5M0B4, Q5XC65, Q83BT3, | |
| | Q2GEI3, Q4QM19, CP000262, Q84FT0, | |
| | Q5M4W1, CP000260, Q1QU94, Q4HIU1, P43844, | |
| | Q40IP4, Q5NFJ3, Q2A498, Q92GH7, Q2GLH3, | |
| | O08370, AY871942, Q68W07, Q4UK96, | |
| | Q4HBL3, Q30P60, Q26C95, Q38WJ7, Q3YRD1, | |
| | P59432, Q7P9P7, JQ1016, P57830, P24531, | |
| | P34894, Q5HW65, Q2X6F1, Q2JFD4, Q2NIT8, | |
| | Q30R29, CP000238, Q6YR37, Q8A9S7, Q5LD58, | |
| | Q5FG30, O51547, Q4HNY8, Q4HFT7, Q8K9P2, | |
| | P57376, Q6AM21, Q3W273, Q660S1, P78011, | |
| | Q6KHH3, Q4A6A3, Q98QM2, Q492D5, | |
| | Q2DZD3, Q89HS7, Q7MAR0, Q7MXW0, | |
| | Q8D253, Q8EWD1, Q7NBH8, Q7VFL1, | |
| | Q4QTL5, P56089, Q3W5W4, Q601P7, Q2E435, | |
| | Q7VRR4, Q4A8E1, P47634, Q4AAB2, Q9ZMP7, | |
| | Q82J74, Q1VNH3, Q50LF3, Q3WZI8, Q9K4E0, | |
| | Q8KJG9, Q98A81, I40886, P50434, Q9W457, | |
| | Q30K91, Q30K95, Q30K92, Q30K98, Q30K94, | |
| | Q30K93, Q5H888, Q29H49, Q1UKA7, Q3KLR8, | |
| | Q6U9U4, Q56F03, Q268J4, Q275S8, Q4I358, | |
| | Q758F0, Q6CLQ5, CH476726, Q94JQ3, T05362, | |
| | Q5L6P4, AJ438778, Q5B0U5, S24342, P07511, | |
| | Q7SXN1, Q2KIP4, Q5E9P9, S65688 | |
| methylene tetrahydrofolate | Cgl2171, EG11585, g1790377 | C. glutamicum, |
| reductase (metF) | | E. coli and |
| | | others |
| cob(I)alamin dependent | Cgl1139, cg1701, CE1637, DIP1259, nfa31930, | |
| methionine synthase I (metH) | Rv2124c, Mb2148c, ML1307, SCO1657, | |
| | Tfu_1825, SAV6667, MT2183, GOX2074, tll1027, | |
| | syc0184_c, alr0308, slr0212, gll0477, SYNW1238, | |
| | TTC0253, TTHA0618, PMT0729, Pro0959, | |
| | PMN2A_0333, PMM0877, WS1234, BH1630, | |
| | GK0716, BCE4332, ABC1869, BC4250, | |
| | BCZK4005, BT9727_3995, BA_4925, | |
| | GBAA4478, BA4478, BAS4156, BLi01192, | |
| | BL01308, MAP1859c, BruAb1_0184, BMEI1759, | |
| | BR0188, SMc03112, MCA1545, AGR_C_3907, | |
| | Atu2155, DR0966, RB9857, ebA3184, VC0390, | |
| | RPA3702, VV11423, VV2960, VP2717, NE1623, | |
| | VF0337, LIC20085, LB108, YPTB3653, | |
| | YPO3722, y0020, YP3084, CV0203, SPA4026, | |
| | MS1009, SC4067, SO1030, DP2202, STM4188, | |
| | STY4405, t4115, PP2375, PFL_3662, Z5610, | |
| | ECs4937, c4976, JW3979, b4019, SF4085, S3645, | |
| | BB4456, BPP3983, BP3594, bll1418, CPS_1101, | |
| | Psyr_2464, PSPTO2732, R03D7.1, PSPPH_2620, | |
| | PBPRA3294, Daro_0046, PA1843, ECA3987, | |
| | CT1857, CAC0578, ACIAD1045, Psyc_0403, | |
| | 4548, DDB0230138, BF3039, BF3199, BT0180, | |
| | 238505, GSU2921, STH2500, XC_2725, | |
| | XCC1511, XOO2073, TTE1803, RSc0294, | |
| | XAC1559, BPSL0385, DVU1585, CTC01806, | |
| | CC2137, TM0268, ZMO1745, FN0163, BG13115, | |
| | lin1786, SAG2048, gbs2004, LMOf2365_1702, | |
| | lmo1678, SE2381, SERP0035, MW0333, | |
| | SAS0333, SMU.874, SA0345, SAV0357, | |
| | SACOL0429, SAR0354, SH2637 | |
| O-acetylhomoserine | NCgl0625, cg0755, CE0679, DIP0630, jk1694, | |
| sulfhydrolase | MAP3457, Mb3372, MT3443, Rv3340, nfa35960, | |
| | Lxx18930, Tfu_2823, CAC2783, GK0284, | |
| | BH2603, lmo0595, lin0604, LMOf2365_0624, | |
| | ABC0432, TTE2151, BT2387, STH2782, str0987, | |
| | stu0987, BF1406, SH0593, BF1342, lp_2536, | |
| | L75975, OB3048, BL0933, LIC11852, LA2062, | |
| | BMAA1890, BPSS0190, SMU.1173, BB1055, | |
| | PP2528, PA5025, PBPRB1415, GSU1183, | |
| | RPA2763, WS1015, TM0882, VP0629, | |
| | BruAb1_0807, BMEI1166, BR0793, CPS_2546, | |
| | XC_1090, XCC3068, plu3517, PMT0875, | |
| | SYNW0851, Pro0800, CT0604, NE1697, RB8221, | |
| | bll1235, syc1143_c, ACIAD3382, ebA6307, | |
| | RSc1562, Daro_2851, DP2506, DR0873, MA2715, | |
| | PMM0642, PMN2A_0083, IL2014, SPO1431, | |
| | ECA0820, AGR_C_2311, Atu1251, mlr8465, | |
| | SMc01809, CV1934, SPBC428.11, PM0738, | |
| | SO1095, SAR11_1030, PFL_0498, CTC01153, | |
| | BA_0514, BCE5535, BAS5258, GBAA5656, | |
| | BA5656, BCZK5104, TTHA0760, TTC0408, | |
| | BC5406, BT9727_5087, HH0636, YLR303W, | |
| | ADL031W, CJE1895, spr1095, rrnAC2716, | |
| | orf19.5645, Cj1727c, VNG2421G, PSPPH_1663, | |
| | XOO1390, Psyr_1669, PSPTO3810, MCA2488, | |
| | TDE2200, FN1419, PG0343, Psyc_0792, MS1347, | |
| | CC3168, Bd3795, MM3085, 389.t00003, | |
| | NMB1609, SAV3305, NMA1808, GOX1671, | |
| | APE1226, XAC3602, NGO1149, ZMO0676, | |
| | SCO4958, lpl0921, lpg0890, lpp0951, EF0290, | |
| | BPP2532, CBU2025, BP3528, BLi02853, | |
| | BL02018, BG12291, CG5345-PA, HP0106, | |
| | ML0275, jhp0098, At3g57050, 107869, HI0086, | |
| | NTHI0100, SpyM3_0133, SPs0136, | |
| | spyM18_0170, M6_Spy0192, SE2323, SERP0095, | |
| | SPy0172, PAB0605, DDB0191318, ST0506, | |
| | F22B8.6, PTO1102, CPE0176, PD1812, XF0864, | |
| | SAR0460, SACOL0503, SA0419, Ta0080, | |
| | PF1266, MW0415, SAS0418, SSO2368, PAE2420, | |
| | TK1449, 1491, TVN0174, PH1093, VF2267, | |
| | Saci_0971, VV11364, CMT389C, VV3008 | |
| aspartate kinase (ask) | Cgl0251, NCgl0247, CE0220, DIP0277, jk1998, | |
| | nfa3180, Mb3736c, MT3812, Rv3709c, ML2323, | |
| | MAP0311c, Tfu_0043, Francci3_0262, SCO3615, | |
| | SAV4559, Lxx03450, PPA2148, CHY_1909, | |
| | MCA0390, cbdb_A1731, TWT708, TW725, | |
| | Gmet_1880, DET1633, GSU1799, Moth_1304, | |
| | Tcr_1589, Mfla_0567, HCH_05208, PSPPH_3511, | |
| | Psyr_3555, PSPTO1843, CV1018, STH1686, | |
| | NMA1701, Tbd_0969, NMB1498, Pcar_1006, | |
| | Daro_2515, Csal_0626, Tmden_1650, PA0904, | |
| | PP4473, Sde_1300, HH0618, NGO0956, | |
| | ACIAD1252, PFL_4505, ebA637, Noc_0927, | |
| | WS1729, Pcryo_1639, Psyc_1461, Pfl_4274, | |
| | LIC12909, LA0693, Rru_A0743, NE2132, | |
| | RB8926, Cj0582, Nmul_A1941, SYN_02781, | |
| | TTHA0534, CJE0685, BURPS1710b_2677, | |
| | BPSL2239, BMA1652, RSc1171, TTC0166, | |
| | RPA0604, BTH_I1945, Bpro_2860, Rmet_1089, | |
| | Reut_A1126, RPD_0099, Bxe_A1630, | |
| | Bcep18194_A5380, aq_1152, RPB_0077, | |
| | Rfer_1353, RPC_0514, BH3096, BLi02996, | |
| | BL00324, amb1612, tlr1833, jhp1150, blr0216, | |
| | Dde_2048, BB1739, BPP2287, BP1913, | |
| | DVU1913, Nwi_0379, ZMO1653, Jann_3191, | |
| | HP1229, Saro_3304, Nham_0472, CBU_1051, | |
| | slr0657, SPO3035, Synpcc7942_1001, BG10350, | |
| | BruAb1_1850, BAB1_1874, BMEI0189, | |
| | BT9727_1658, syc0544_d, BR1871, gll1774, | |
| | BC1748, mll3437, BCE1883, ELI_14545, | |
| | RSP_1849, BCZK1623, BAS1676, BA_2315, | |
| | GBAA1811, BA1811, Ava_3642, alr3644, | |
| | PSHAa0533, AGR_L_1357, Atu4172, lin1198, | |
| | BH04030, PMT9312_1740, SMc02438, | |
| | CYA_1747, RHE_CH03758, lmo1235, | |
| | LMOf2365_1244, PMN2A_1246, CC0843, | |
| | Pro1808, BQ03060, PMT0073, Syncc9902_0068, | |
| | GOX0037, CYB_0217 | |
| homoserine dehydrogenase | Cgl1183, cg1337, NCgl1136, CE1289, DIP1036, | |
| (hom) | jk1352, nfa10490, SAV2918, Mb1326, MT1333, | |
| | Rv1294, SCO5354, MAP2468c, ML1129, | |
| | Francci3_3725, Tfu_2424, Lxx06870, PPA1258, | |
| | Moth_1307, BL1274, CHY_1912, DSY1363, | |
| | GK2964, CAC0998, BLi03414, BL02137, | |
| | BC5404, STH2739, BCZK5102, BT9727_5085, | |
| | Gmet_1629, BCE5533, BB1926, BP2784, | |
| | CTC02355, BG10460, BPP2479, BAS5256, | |
| | BA_0512, GBAA5654, BA5654, | |
| | Synpcc7942_2090, syc2003_c, Adeh_1638, | |
| | CYA_1100, Pcar_1451, Mfla_1048, Mfla_0904, | |
| | TW329, TWT439, BH3422, all4120, Daro_2386, | |
| | gll4295, ebA4952, Ava_0783, Syncc9605_1957, | |
| | LSL_1519, OB0466, lmo2547, PMT1143, | |
| | Bpro_2190, SYNW0711, LMOf2365_2520, | |
| | lin2691, sll0455, CV0996, RSc1327, | |
| | PMT9312_1062, ABC2942, Bcep18194_A5155, | |
| | BURPS1710b_2396, BPSL1477, BMA1385, | |
| | NMA1395, NMB1228, tll0277, Syncc9902_0704, | |
| | GSU1693, Bxe_A2381, MCA0597, NGO0779, | |
| | CYB_1425, BTH_I2198, BMEI0725, Rmet_1966, | |
| | Rfer_1912, SMc00293, BruAb1_1275, | |
| | BAB1_1293, SYN_00890, Reut_A1993, | |
| | RHE_CH01878, BR1274, aq_1812, TTE2620, | |
| | ACIAD0264, PFL_1103, stu0469, str0469, | |
| | Pfl_1027, Psyr_1290, PMN2A_0702, MTH1232, | |
| | Csal_3010, AGR_C_2919, Atu1588, PSPPH_1360, | |
| | PP1470, NE2369, PSPTO1480, Tcr_1251, | |
| | BC1964, Nmul_A1551, Saro_0019, mll0934, | |
| | WS0450, spr1219, SP1361, Noc_2454, | |
| | BT9727_1799, BCZK1782, BCE2051, Tbd_0843, | |
| | PA3736, DET1206, amb3728, Rru_A2410, | |
| | LIC10571, LA3638, SMU.965, BAS1825, | |
| | BA_2468, GBAA1968, BA1968, cbdb_A1123, | |
| | GOX1517, PMM1051, HCH_01779, RB8510, | |
| | DVU0890, Pro1150, Nham_2309, Tmden_1904, | |
| | Sde_1209, Psyc_0253, ELI_13775, RSP_0403, | |
| | L0090, Dde_2731, Pcryo_0279, Nwi_1647, | |
| | lp_0571, BH10030, SPO1734, Jann_2998, blr4362, | |
| | RPA2504, EF2422, DP1732, LBA1212, | |
| | RPD_2495, RPC_2816, CC1383, RPB_2966, | |
| | CJE0145, Cj0149c, Acid345_1481, ZMO0483, | |
| | Bpro_5333, SAK_1205, gbs1187, jhp0761, | |
| | SH1579, SAG1120, HP0822, SE1009, SERP0897, | |
| | SAOUHSC_01320, SAUSA300_1226, SAB1186, | |
| | SACOL1362, SAS1268, SAR1338, MW1215, | |
| | SAV1328, SA1164, HH1750, SSP1438, lp_2535, | |
| | TTE2152, SAR11_1025, DR1278, PFL_3809, | |
| | Dgeo_0610, Mhun_2292, DSY3981, PP0664, | |
| | MA2572, ABC1578, Mbar_A1898, TTHA0489, | |
| | TTC0115, MM2713, Mbur_1087, BH1737, | |
| | AF0935, MK1554, MTH417, VNG2650G, | |
| | Msp_0487, ABC0023, rrnAC2408, TK1627, | |
| | TM0547, MJ1602, NP0302A, BH1253, MMP1702, | |
| | BCE2626, LmjF07.0260, BCZK2354, | |
| | BT9727_2388, BAS2433, BA_3119, GBAA2608, | |
| | BA2608, BC2548, Acid345_4165, CTC00886, | |
| | ST1519, Saci_1636, APE1144, SSO0657, PF1104, | |
| | Adeh_3931, PAB0610, PH1075, Cag_0142, | |
| | PAE2868, YJR139C, XOO1820, Plut_1983, | |
| | XAC3038, Adeh_1400, XCV3175, PTO1417, | |
| | SCO0420, SRU_0482, XC_1253, XCC2855, | |
| | SO4055, CT2030, SPBC776.03, | |
| | AO090003000721, TVN0385, ABL080W, | |
| | AO090009000136, CPS_0456, HI0089, | |
| | orf19.2951, Sden_0616, UTI89_C4525, | |
| | Afu3g11640, MS1703, SBO_3960, SSO_4114, | |
| | STM4101, SC3992, t3517, STY3768, c4893, | |
| | ECs4869, Z5495, JW3911, b3940, AN2882.2, | |
| | ECA4251, CMN129C, NTHI0167, plu4755, | |
| | ECA3891, YPTB0602, YP3723, y3718, YPO0459, | |
| | PM0113, S3729, SF4018, SPA3944, Mfla_1298, | |
| | PSHAa2379, PBPRA0262, XOO2242, STM0002, | |
| | SC0002, SPA0002, t0002, STY0002, c0003, | |
| | SRU_0691, XCC1800, PD1273, BPEN_115, | |
| | SDY_3775, VC2684, SDY_0002, SBO_0001, | |
| | YPTB0106, YP0118, y0303, YPO0116, | |
| | UTI89_C0002, ECs0002, Z0002, JW0001, b0002, | |
| | VV3007, VV11365, XC_2389, VP2764, XF2225, | |
| | SSO_0002, S0002, SF0002 | |
| Serine deaminase (sda) | GeneID: 1019614, NCgl1583, EG10930, g178116 | C. glutamicum, |
| | | E. coli, and |
| | | others |
| Homoserine kinase (hsk) | Cgl1184, cg0307, CE0221, DIP0279, jk1997, RHA1_ro04292, | C. glutamicum |
| | nfa3190, Mmcs_4888, | and others |
| | MSMEG_6256, MAP0310c, MAV_0394, Mb3735c, | |
| | MT3811, Rv3708c, Acel_2011, ML2322, PPA0318, Lxx03460, | |
| | SCO2640, SAV5397, CC3485 | |
| D-methionine binding | YP_224930, NP_599871, NP_737241, NP_938985, | C. glutamicum |
| lipoprotein (metQ) | NP_938984, YP_701727, YP_251505, YP_120623, | and others |
| | YP_062481, YP_056445, ZP_00121548, NP_696133, | |
| | YP_034633, YP_034633, YP_081895, ZP_00390696, | |
| | YP_016928, YP_026579, NP_842863, YP_081895, | |
| | ZP_00240243, NP_976671 | |
| mcbR | cg3253, CE2788, DIP2274, jk0101, nfa21280, MSMEG_4517Lxx16190, | C. glutamicum |
| | SCO4454, Bcep18194_A3587, Bamb_0404, | and others |
| | Bcen2424_0499, Bcen_2606, Ava_4037, | |
| | BTH_I2940, RHA1_ro02712, BMA10299_A1735, BMASAVP1_A0031, | |
| | BMA2807, BURPS1710b_3614 | |
| Glucose-6-phosphate- | Cgl1576, BAB98969, NCgl1514, NCgl1514, cgl778, | Corynebacterium |
| dehydrogenase | CE1696, DIP1304, jk0994, RHA1_ro07184, nfa35750, | glutamicum |
| | MSMEG_3101, Mmcs_2412, MAP1176c, Mb1482c, | and others |
| | MT1494, Rv1447c, SAV6313, Acel_1124, SCO1937, | |
| | MAV_3329, Lxx11590, BL0440, Arth_2094, Tfu_2005, | |
| | itte weitere angeben | |
| OPCA protein | Cgl1577, NP_738307.1, NP_939658.1, YP_250777.1, | Corynebacterium |
| | YP_707105.1, YP_119788.1, ZP_01192082.1, NP_335942.1, | glutamicum |
| | ZP_01276169.1, NP_215962.1, ZP_01684361.1, | and others |
| | YP_887415.1, ZP_01130849.1, YP_062111.1, | |
| | ZP_00615668.1, YP_953530.1, ZP_00995403.1, YP_882512.1, | |
| | NP_960109.1, YP_290062.1, YP_831573.1, | |
| | NP_827488.1, YP_947837.1, NP_822945.1, NP_626203.1, | |
| | NP_630735.1, CAH10103.1, ZP_00120910.2, | |
| | NP_695642.1, YP_909493.1, YP_872881.1, YP_923728.1, | |
| | YP_056265.1, ZP_01648612.1, ZP_01430762.1, | |
| | ZP_00569428.1, YP_714762.1, YP_480751.1, | |
| | NP_301492.1, YP_642845.1, ZP_00767699.1 | |
| Transaldolase | Cgl1575, cg1776, CE1695, DIP1303, jk0993, Mmcs_2413, | Corynebacterium |
| | MSMEG_3102, MAP1177c, RHA1_ro07185, | glutamicum |
| | MAV_3328, Mb1483c, Rv1448c, MT1495, nfa35740, | and others |
| | ML0582, Arth_2096, Lxx11610, SAV1767, Tfu_2003, | |
| | SCO1936, Francci3_1648 | |
| 6-phosphogluconolactonase | Cgl1578, NCgl1516, NCgl1516, cg1780, CE1698, DIP1306, | Corynebacterium |
| | Mmcs_2410, MSMEG_3099, Mb1480c, MT1492, | glutamicum |
| | Rv1445c, MAV_3331, RHA1_ro07182, nfa35770, | and others |
| | MAP1174c, ML0579, jk0996, Tfu_2007, FRAAL4578, | |
| | SAV6311, SCO1939, SCC22.21, TW464 | |
| Transketolase | Cgl1574, YP_225858, cg1774, CE1694, DIP1302, jk0992, | Corynebacterium |
| | nfa35730, RHA1_ro07186, MSMEG_3103, MAP1178c, | glutamicum |
| | ML0583, MAV_3327, Mb1484c, MT1496, Rv1449c, | and others |
| | Mmcs_2414, Tfu_2002, Arth_2097, Lxx11620, | |
| | SAV1766, SCO1935, Acel_1127 | |

The above accession numbers are the official accession numbers of Genbank or are synonyms for accession numbers which have cross-references at Genbank. These numbers can be searched and found at http://www.ncbi.nlm.nih.gov/.

A general overview is given below how to increase and decrease the amount and/or activity of polypeptides and genes in C. glutamicum and E. coli. Nevertheless, the person skilled in the art will be aware of other technologies and approaches for either identifying new homologs of the enzymes of Table 1 by performing appropriate database searches and/or altering the expression of these enzymes in organisms other than Coryneform bacteria or bacteria of the genus Escherichia.

Increasing or Introducing the Amount and/or Activity

With respect to increasing the amount, two basic scenarios can be differentiated. In the first scenario, the amount of the enzyme is increased by expression of an exogenous version of the respective protein. In the other scenario, expression of the endogenous protein is increased by influencing the activity of e.g. the promoter and/or enhancers element and/or other regulatory activities that regulate the activities of the respective proteins either on a transcriptional, translational or post-translational level.

Thus, the increase of the activity and the amount of a protein may be achieved via different routes, e.g. by switching off inhibitory regulatory mechanisms at the transcriptional, translational, and protein level or by increase of gene expression of a nucleic acid coding for these proteins in comparison with the starting organism, e.g. by inducing endogenous transketolase by a strong promoter and/or by introducing nucleic acids encoding for transketolase.

In one embodiment, the increase of the amount and/or activity of the enzymes of Table 1 is achieved by introducing nucleic acids encoding the enzymes of Table 1 into microorganism such as C. glutamicum and E. coli.

In principle, any protein of different organisms with an enzymatic activity of the proteins listed in Table 1 can be used. With genomic nucleic acid sequences of such enzymes from eukaryotic sources containing introns, already processed nucleic acid sequences like the corresponding cDNAs are to be used in the case as the host organism is not capable or cannot be made capable of splicing the corresponding mRNAs. All nucleic acids mentioned in the description can be, e.g., an RNA, DNA or cDNA sequence.

According to the present invention, increasing or introducing the amount of a protein typically comprises the following steps:

a) production of a vector comprising the following nucleic acid sequences, preferably DNA sequences, in 5′-3′-orientation:
a promoter sequence functional in an organism of the invention
operatively linked thereto a DNA sequence coding for a protein of e.g. Table 1, functional homologues, functional fragments or functional mutated versions thereof
optionally, a termination sequence functional in the organisms of the invention
   b) transfer of the vector from step a) to an organisms of the invention such as C. glutamicum and, optionally, integration into the respective genomes.

As set out above, functional fragments relate to fragments of nucleic acid sequences coding for enzymes of e.g. Table 1, the expression of which still leads to proteins having the enzymatic activity substantially similar to that of the respective full length protein.

The above-mentioned method can be used for increasing the expression of DNA sequences coding for enzymes of e.g. Table 1 or functional fragments thereof. The use of such vectors comprising regulatory sequences, like promoter and termination sequences are, is known to the person skilled in the art. Furthermore, the person skilled in the art knows how a vector from step a) can be transferred to organisms such as C. glutamicum or E. coli and which properties a vector must have to be able to be integrated into their genomes.

If the enzyme content in an organism such as C. glutamicum is increased by transferring a nucleic acid coding for an enzyme from another organism, like e.g. E. coli, it is advisable to transfer the amino acid sequence encoded by the nucleic acid sequence e.g. from E. coli by back-translation of the polypeptide sequence according to the genetic code into a nucleic acid sequence comprising mainly those codons, which are used more often due to the organism-specific codon usage. The codon usage can be determined by means of computer evaluations of other known genes of the relevant organisms.

According to the present invention, an increase of the gene expression of a nucleic acid encoding an enzyme of Table 1 is also understood to be the manipulation of the expression of the endogenous respective endogenous enzymes of an organism, in particular of C. glutamicum. This can be achieved, e.g., by altering the promoter DNA sequence for genes encoding these enzymes. Such an alteration, which causes an altered, preferably increased, expression rate of these enzymes can be achieved by replacement with strong promoters and by deletion and/or insertion of DNA sequences.

An alteration of the promoter sequence of endogenous genes usually causes an alteration of the expressed amount of the gene and therefore also an alteration of the activity detectable in the cell or in the organism.

Furthermore, an altered and increased expression, respectively, of an endogenous gene can be achieved by a regulatory protein, which does not occur or has been deleted in the transformed organism, and which interacts with the promoter of these genes. Such a regulator can be a chimeric protein consisting of a DNA binding domain and a transcription activator domain, as e.g. described in WO 96/06166.

A further possibility for increasing the activity and the content of endogenous genes is to up-regulate transcription factors involved in the transcription of the endogenous genes, e.g. by means of overexpression. The measures for overexpression of transcription factors are known to the person skilled in the art.

The expression of endogenous enzymes such as those of Table 1 can e.g. be regulated via the expression of aptamers specifically binding to the promoter sequences of the genes. Depending on the aptamer binding to stimulating or repressing promoter regions, the amount of the enzymes of Table 1 can e.g. be increased.

Furthermore, an alteration of the activity of endogenous genes can be achieved by targeted mutagenesis of the endogenous gene copies.

An alteration of the endogenous genes coding for the enzymes of e.g. Table 1 can also be achieved by influencing the post-translational modifications of the enzymes. This can happen e.g. by regulating the activity of enzymes like kinases or phosphatases involved in the post-translational modification of the enzymes by means of corresponding measures like overexpression or gene silencing.

In another embodiment, an enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired enzyme of Table 1 without impairing the viability of the cell. In each case, the overall yield, rate of production or amount of methionine be increased.

These aforementioned strategies for increasing or introducing the amount and/or activity of the enzymes of Table 1 are not meant to be limiting; variations on these strategies will be readily apparent to one of ordinary skill in the art.

Reducing the Amount and/or Activity of Enzymes

It has been set out above that it may be preferred to use a starting organism which have already been engineered for methionine production. In C. glutamicum one may, for example, downregulate the activity of metQ.

For reducing the amount and/or activity of enzymes, various strategies are available.

The expression of endogenous enzymes such as those of Table 1 can e.g. be regulated via the expression of aptamers specifically binding to the promoter sequences of the genes. Depending on the aptamer binding to stimulating or repressing promoter regions, the amount and thus, in this case, the activity of the enzymes of Table 1 can e.g. be reduced.

Aptamers can also be designed in a way as to specifically bind to the enzymes themselves and to reduce the activity of the enzymes by e.g. binding to the catalytic center of the respective enzymes. The expression of aptamers is usually achieved by vector-based overexpression (see above) and is, as well as the design and the selection of aptamers, well known to the person skilled in the art (Famulok et al., (1999) Curr Top Microbiol Immunol., 243, 123-36).

Furthermore, a decrease of the amount and the activity of the endogenous enzymes of Table 1 can be achieved by means of various experimental measures, which are well known to the person skilled in the art. These measures are usually summarized under the term “gene silencing”. For example, the expression of an endogenous gene can be silenced by transferring an above-mentioned vector, which has a DNA sequence coding for the enzyme or parts thereof in antisense order, to organisms such as C. glutamicum. This is based on the fact that the transcription of such a vector in the cell leads to an RNA, which can hybridize with the mRNA transcribed by the endogenous gene and therefore prevents its translation.

In principle, the antisense strategy can be coupled with a ribozyme method. Ribozymes are catalytically active RNA sequences, which, if coupled to the antisense sequences, cleave the target sequences catalytically (Tanner et al., (1999) FEMS Microbiol Rev. 23 (3), 257-75). This can enhance the efficiency of an antisense strategy.

To create a homologous recombinant microorganism, a vector is prepared which contains at least a portion of gene coding for an enzyme of Table 1 into which a deletion, addition or substitution has been introduced to thereby alter, e.g., functionally disrupt, the endogenous gene.

In one embodiment, the vector is designed such that, upon homologous recombination, the endogenous gene is functionally disrupted (i.e., no longer encodes a functional protein). Alternatively, the vector can be designed such that, upon homologous recombination, the endogenous gene is mutated or otherwise altered but still encodes functional protein, e.g., the upstream regulatory region can be altered to thereby alter the expression of the endogenous enzymes of Table 1. This approach can have the advantage that expression of an enzyme is not completely abolished, but reduced to the required minimum level. The skilled person knows which vectors can be used to replace or delete endogenous sequences. A specific description for disrupting chromosomal sequences in C. glutamicum is provided below.

Furthermore, gene repression is possible by reducing the amount of transcription factors.

Factors inhibiting the target protein itself can also be introduced into a cell. The protein-binding factors may e.g. be the above-mentioned aptamers (Famulok et al., (1999) Curr Top Microbiol Immunol. 243, 123-36).

As further protein-binding factors, the expression of which can cause a reduction of the amount and/or the activity of the enzymes of table 1, enzyme-specific antibodies may be considered. The production of recombinant enzyme-specific antibodies such as single chain antibodies is known in the art. The expression of antibodies is also known from the literature (Fiedler et al., (1997) Immunotechnology 3, 205-216; Maynard and Georgiou (2000) Annu. Rev. Biomed. Eng. 2, 339-76).

The mentioned techniques are well known to the person skilled in the art. Therefore, the skilled also knows the typical size that a nucleic acid constructs used for e.g. antisense methods must have and which complementarity, homology or identity, the respective nucleic acid sequences must have. The terms complementarity, homology, and identity are known to the person skilled in the art.

The term complementarity describes the capability of a nucleic acid molecule to hybridize with another nucleic acid molecule due to hydrogen bonds between two complementary bases. The person skilled in the art knows that two nucleic acid molecules do not have to display a complementarity of 100% in order to be able to hybridize with each other. A nucleic acid sequence, which is to hybridize with another nucleic acid sequence, is preferably at least 30%, at least 40%, at least 50%, at least 60%, preferably at least 70%, particularly preferred at least 80%, also particularly preferred at least 90%, in particular preferred at least 95% and most preferably at least 98 or 100%, respectively, complementary with said other nucleic acid sequence.

The hybridization of an antisense sequence with an endogenous mRNA sequence typically occurs in vivo under cellular conditions or in vitro. According to the present invention, hybridization is carried out in vivo or in vitro under conditions that are stringent enough to ensure a specific hybridization.

Stringent in vitro hybridization conditions are known to the person skilled in the art and can be taken from the literature (see e.g. Sambrook et al., Molecular Cloning, Cold Spring Harbor Press (2001)). The term “specific hybridization” refers to the case wherein a molecule preferentially binds to a certain nucleic acid sequence under stringent conditions, if this nucleic acid sequence is part of a complex mixture of e.g. DNA or RNA molecules.

The term “stringent conditions” therefore refers to conditions, under which a nucleic acid sequence preferentially binds to a target sequence, but not, or at least to a significantly reduced extent, to other sequences.

Stringent conditions are dependent on the circumstances. Longer sequences specifically hybridize at higher temperatures. In general, stringent conditions are chosen in such a way that the hybridization temperature lies about 5° C. below the melting point (Tm) of the specific sequence with a defined ionic strength and a defined pH value. Tm is the temperature (with a defined pH value, a defined ionic strength and a defined nucleic acid concentration), at which 50% of the molecules, which are complementary to a target sequence, hybridize with said target sequence.

Typically, stringent conditions comprise salt concentrations between 0.01 and 1.0 M sodium ions (or ions of another salt) and a pH value between 7.0 and 8.3. The temperature is at least 30° C. for short molecules (e.g. for such molecules comprising between 10 and 50 nucleic acids). In addition, stringent conditions can comprise the addition of destabilizing agents like e.g. form amide. Typical hybridization and washing buffers are of the following composition.

| | |
|---|---|
| Pre-hybridization solution: | 0.5% SDS |
| | 5x SSC |
| | 50 mM NaPO₄, pH 6.8 |
| | 0.1% Na-pyrophosphate |
| | 5x Denhardt's reagent |
| | 100 μg/salmon sperm |
| Hybridization solution: | Pre-hybridization solution |
| | 1 × 10⁶ cpm/ml probe (5-10 min 95° C.) |
| 20x SSC: | 3 M NaCl |
| | 0.3 M sodium citrate |
| | ad pH 7 with HCl |
| 50x Denhardt's reagent: | 5 g Ficoll |
| | 5 g polyvinylpyrrolidone |
| | 5 g Bovine Serum Albumin |
| | ad 500 ml A. dest. |

A typical procedure for the hybridization is as follows:

| Optional: | wash Blot 30 min in 1x SSC/0.1% SDS at 65° C. | | |
|---|---|---|---|
| Pre-hybridization: | at least 2 h at 50-55° C. | | |
| Hybridization: | over night at 55-60° C. | | |
| Washing: | 05 min | 2x SSC/0.1% SDS | |
| | Hybridization temperature | | |
| | 30 min | 2x SSC/0.1% SDS | |
| | Hybridization temperature | | |
| | 30 min | 1x SSC/0.1% SDS | |
| | Hybridization temperature | | |
| | 45 min | 0.2x SSC/0.1% SDS | 65° C. |
| | 5 min | 0.1x SSC | room temperature |

For antisense purposes complementarity over sequence lengths of 100 nucleic acids, 80 nucleic acids, 60 nucleic acids, 40 nucleic acids and 20 nucleic acids may suffice. Longer nucleic acid lengths will certainly also suffice. A combined application of the above-mentioned methods is also conceivable.

If, according to the present invention, DNA sequences are used, which are operatively linked in 5′-3′-orientation to a promoter active in the organism, vectors can, in general, be constructed, which, after the transfer to the organism's cells, allow the overexpression of the coding sequence or cause the suppression or competition and blockage of endogenous nucleic acid sequences and the proteins expressed there from, respectively.

The activity of a particular enzyme may also be reduced by over-expressing a non-functional mutant thereof in the organism. Thus, a non-functional mutant which is not able to catalyze the reaction in question, but that is able to bind e.g. the substrate or co-factor, can, by way of over-expression out-compete the endogenous enzyme and therefore inhibit the reaction. Further methods in order to reduce the amount and/or activity of an enzyme in a host cell are well known to the person skilled in the art.

According to the present invention, non-functional enzymes have essentially the same nucleic acid sequences and amino acid sequences, respectively, as functional enzymes and functionally fragments thereof, but have, at some positions, point mutations, insertions or deletions of nucleic acids or amino acids, which have the effect that the non-functional enzyme are not, or only to a very limited extent, capable of catalyzing the respective reaction. These non-functional enzymes may not be intermixed with enzymes that still are capable of catalyzing the respective reaction, but which are not feedback regulated anymore. According to the present invention, the term “non-functional enzyme” does not comprise such proteins having no substantial sequence homology to the respective functional enzymes at the amino acid level and nucleic acid level, respectively. Proteins unable to catalyse the respective reactions and having no substantial sequence homology with the respective enzyme are therefore, by definition, not meant by the term “non-functional enzyme” of the present invention. Non-functional enzymes are, within the scope of the present invention, also referred to as inactivated or inactive enzymes.

Therefore, non-functional enzymes of e.g. Table 1 according to the present invention bearing the above-mentioned point mutations, insertions, and/or deletions are characterized by an substantial sequence homology to the wild type enzymes of e.g. Table 1 according to the present invention or functionally equivalent parts thereof. For determining a substantial sequence homology, the above described identity grades are to applied.

### Vectors and Host Cells

One aspect of the invention pertains to vectors, preferably expression vectors, containing a modified nucleic acid sequences as mentioned above. As used herein, the term “vector” refers to a nucleic acid molecule capable of transporting another nucleic acid to which it has been linked.

One type of vector is a “plasmid”, which refers to a circular double stranded DNA loop into which additional DNA segments can be ligated. Another type of vector is a viral vector, wherein additional DNA segments can be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked.

Such vectors are referred to herein as “expression vectors”.

In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention may comprise a modified nucleic acid as mentioned above in a form suitable for expression of the respective nucleic acid in a host cell, which means that the recombinant expression vectors include one or more regulatory sequences, selected on the basis of the host cells to be used for expression, which is operatively linked to the nucleic acid sequence to be expressed.

Within a recombinant expression vector, “operably linked” is intended to mean that the nucleic acid sequence of interest is linked to the regulatory sequence (s) in a manner which allows for expression of the nucleic acid sequence (e.g., in an in vitro transcription/translation system or in a host cell when the vector is introduced into the host cell). The term “regulatory sequence” is intended to include promoters, repressor binding sites, activator binding sites, enhancers and other expression control elements (e.g., terminators, polyadenylation signals, or other elements of mRNA secondary structure). Such regulatory sequences are described, for example, in Goeddel; Gene Expression Technology: Methods in Enzymology 185, Academic Press, San Diego, Calif. (1990). Regulatory sequences include those which direct constitutive expression of a nucleic acid sequence in many types of host cell and those which direct expression of the nucleic acid sequence only in certain host cells. Preferred regulatory sequences are, for example, promoters such as cos-, tac-, trp-, tet-, trp-, tet-, lpp-, lac-, lpp-lac-, lacIq-, T7-, T5-, T3-, gal-, trc-, ara-, SP6-, arny, SP02, phage lambdaP_{R}, phage lambdaP_{L}, phage SP01 P₁₅, phage SP01 P₂₆, pSOD, EFTu, EFTs, GroEL, MetZ (last 5 from C. glutamicum), which are used preferably in bacteria. Additional regulatory sequences are, for example, promoters from yeasts and fungi, such as ADC1, MFa, AC, P-60, CYC1, GAPDH, TEF, rp28, ADH, ENO2, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by the above-mentioned modified nucleic acid sequences.

Expression of proteins in prokaryotes is most often carried out with vectors containing constitutive or inducible promoters directing the expression of either fusion or non-fusion proteins.

Fusion vectors add a number of amino acids to a protein encoded therein, usually to the amino terminus of the recombinant protein but also to the C-terminus or fused within suitable regions in the proteins. Such fusion vectors typically serve four purposes: 1) to increase expression of recombinant protein; 2) to increase the solubility of the recombinant protein; and 3) to aid in the purification of the recombinant protein by acting as a ligand in affinity purification 4) to provide a “tag” for later detection of the protein. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively.

Examples of suitable inducible non-fusion E. coli expression vectors include pTrc (Amann et al., (1988) Gene 69: 301-315), pLG338, pACYC184, pBR322, pUC18, pUC19, pKC30, pRep4, pHS1, pHS2, pPLc236, pMBL24, pLG200, pUR290, pIN-III113-B1, egtll, pBdCl, and pET lld (Studier et al., Gene Expression Technology: Methods in Enzymology 185, Academic Press, San Diego, Calif. (1990) 60-89; and Pouwels et al., eds. (1985) Cloning Vectors. Elsevier: New York IBSN 0 444 904018). Target gene expression from the pTrc vector relies on host RNA polymerase transcription from a hybrid trp-lac fusion promoter. Target gene expression from the pET lld vector relies on transcription from a T7 gnlO-lac fusion promoter mediated by a coexpressed viral RNA polymerase (T7gnl). This viral polymerase is supplied by host strains BL21 (DE3) or HMS174 (DE3) from a resident X prophage harboring a T7gnl gene under the transcriptional control of the lacUV 5 promoter. For transformation of other varieties of bacteria, appropriate vectors may be selected. For example, the plasmids pIJ101, pIJ364, pIJ702 and pIJ361 are known to be useful in transforming Streptomyces, while plasmidspUB110, pC194 or pBD214 are suited for transformation of Bacillus species. Several plasmids of use in the transfer of genetic information into Corynebacterium include pHM1519, pBL1, pSA77 or pAJ667 (Pouwels et al., eds. (1985) Cloning Vectors. Elsevier: New York IBSN 0 444 904018).

Examples of suitable C. glutamicum and E coli shuttle vectors are e.g. pClik5aMCS (WO2005059093) or can be found in Eikmanns et al (Gene. (1991) 102, 93-8).

Examples for suitable vectors to manipulate Corynebacteria can be found in the Handbook of Corynebacterium (edited by Eggeling and Bott, ISBN 0-8493-1821-1, 2005). One can find a list of E. coli-C. glutamicum shuttle vectors (table 23.1), a list of E. coli-C. glutamicum shuttle expression vectors (table 23.2), a list of vectors which can be used for the integration of DNA into the C. glutamicum chromosome (table 23.3), a list of expression vectors for integration into the C. glutamicum chromosome (table 23.4.) as well as a list of vectors for site-specific integration into the C. glutamicum chromosome (table 23.6).

In another embodiment, the protein expression vector is a yeast expression vector. Examples of vectors for expression in yeast S. cerevisiae include pYepSec1 (Baldari, et al., (1987) Embo J. 6: 229-234), 21, pAG-1, Yep6, Yep13, pEMBLYe23, pMFa (Kurjan and Herskowitz, (1982) Cell 30: 933-943), pJRY88 (Schultz et al., (1987) Gene 54: 113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) in: Applied Molecular Genetics of Fungi, J. F. Peberdy, et al., eds., p. 1-28, Cambridge University Press: Cambridge, and Pouwels et al., eds. (1985) Cloning Vectors. Elsevier: New York (IBSN 0 444 904018).

For the purposes of the present invention, an operative link is understood to be the sequential arrangement of promoter, coding sequence, terminator and, optionally, further regulatory elements in such a way that each of the regulatory elements can fulfill its function, according to its determination, when expressing the coding sequence.

For other suitable expression systems for both prokaryotic and eukaryotic cells see chapters 16 and 17 of Sambrook, J. et al. Molecular Cloning: A Laboratory Manual. 3rd ed., Cold Spring Harbor Laboratory, Cold Spring Harbor Laboratory Press, Cold Spring Harbor, N.Y., 2003.

Vector DNA can be introduced into prokaryotic via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA into a host cell, including calcium phosphate or calcium chloride co-precipitation, DEAE-dextran-mediated transfection, lipofection, natural competence, chemical-mediated transfer, or electroporation. Suitable methods for transforming or transfecting host cells can be found in Sambrook, et al. (Molecular Cloning: A Laboratory Manual. 3rd ed., Cold Spring Harbor Laboratory, Cold Spring Harbor Laboratory Press, Cold Spring Harbor, N.Y., 2003), and other laboratory manuals.

In order to identify and select these integrants, a gene that encodes a selectable marker (e.g., resistance to antibiotics) is generally introduced into the host cells along with the gene of interest. Preferred selectable markers include those which confer resistance to drugs, such as, but not limited to, G418, hygromycin, kanamycine, tetracycline, neomycineampicillin (and other pencillins), cephalosporins, fluoroquinones, naladixic a id, chloramphenicol, spectinomyin, ertythromycin, streptomycin and methotrexate. Other selectable markers include wild type genes that can complement mutated versions of the equivalent gene in a host or starting strain. For example, an essential gene for growth on a minimal medium, such as serA, can be mutated or deleted from the genome of a C. glutamicum starting or host strain of the invention as described herein above to create a serine auxotroph. Then, a vector containing a wild type or other functional copy of a serA gene can be used to select for transformants or integrants. Nucleic acid encoding a selectable marker can be introduced into a host cell on the same vector as that encoding the above-mentioned modified nucleic acid sequences or can be introduced on a separate vector. Cells stably transfected with the introduced nucleic acid can be identified by drug selection (e.g., cells that have incorporated the selectable marker gene will survive, while the other cells die).

When plasmids without an origin of replication and two different marker genes are used (e.g. pClik int sacB), it is also possible to generate marker-free strains which have part of the insert inserted into the genome. This is achieved by two consecutive events of homologous recombination (see also Becker et al., Applied and Environmental Microbiology, 71 (12), p. 8587-8596). The sequence of plasmid pClik int sacB can be found in WO2005059093; SEQ ID 24; the plasmid is called pCIS in this document.

In another embodiment, recombinant microorganisms can be produced which contain selected systems which allow for regulated expression of the introduced gene. For example, inclusion of one of the above-mentioned nucleic acid sequences on a vector placing it under control of the lac operon permits expression of the gene only in the presence of IPTG. Such regulatory systems are well known in the art.

Another aspect of the invention pertains to organisms or host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Growth of E. coli and C. glutamicum—Media and Culture Conditions

The person skilled in the art is familiar with the cultivation of common microorganisms such as C. glutamicum and E. coli. Thus, a general teaching will be given below as to the cultivation of C. glutamicum. Corresponding information may be retrieved from standard textbooks for cultivation of E. coli.

E. coli strains are routinely grown in MB and LB broth, respectively (Follettie et al. (1993) J. Bacteriol. 175, 4096-4103). Minimal Several minimal media for bacteria, including E. coli and C. glutamicum are well known in the art. Minimal media for E. coli is include, but are not limited to, E medium, M9 medium and modified MCGC (Yoshihama et al. (1985) J. Bacteriol. 162, 591-507), respectively. Glucose may be added at a final concentration of between about 0.2% and 1%. Antibiotics may be added in the following amounts (micrograms per millilitre): ampicillin, 5 to 1000; kanamycin, 25; nalidixic acid, 25; chloramphenicol, 5 to 120, spectinomycin 50 to 100, tetracyline 5 to 120. Amino acids, vitamins, and other supplements may be added, for example, in the following amounts: methionine, 9.3 mM; arginine, 9.3 mM; histidine, 9.3 mM; thiamine, 0.05 mM. E. coli cells are routinely grown at 18 to 37 44° C., respectively depending on the particular experiment or procedure being performed.

Genetically modified Corynebacteria are typically cultured in synthetic or natural growth media. A number of different growth media for Corynebacteria are both well-known and readily available (Lieb et al. (1989) Appl. Microbiol. Biotechnol., 32: 205-210; von der Osten et al. (1998) Biotechnology Letters, 11: 11-16; Patent DE 4,120,867; Liebl (1992) “The Genus Corynebacterium, in: The Procaryotes, Volume II, Balows, A. et al., eds. Springer-Verlag). Instructions can also be found in the Handbook of Corynebacterium (edited by Eggeling and Bott, ISBN 0-8493-1821-1, 2005).

These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribose, lactose, maltose, sucrose, glycerol, raffinose, starch or cellulose serve as very good carbon sources.

It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

The overproduction of methionine is possible using different sulfur sources. Sulfates, thiosulfates, sulfites and also more reduced sulfur sources like H₂S and sulfides and derivatives can be used. Also organic sulfur sources like methyl mercaptan, thioglycolates, thiocyanates, and thiourea, sulfur containing amino acids like cysteine and other sulfur containing compounds can be used to achieve efficient methionine production. Formate may also be possible as a supplement as are other C1 sources such as methanol or formaldehyde.

Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include cyanocobalamin (or other form of vitamin B12), biotin, riboflavin, thiamine, folic acid, nicotinic acid, pantothenate and pyridoxine. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (Eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components should be sterilized, either by heat (20 minutes at 1.5 bar and 121 C) or by sterile filtration. The components can either be sterilized together or, if necessary, separately.

All media components may be present at the beginning of growth, or they can optionally be added continuously or batch wise. Culture conditions are defined separately for each experiment.

The temperature should be is usually in a range between 15° C. and 45° C., but the range may be higher, up to 105° C. for thermophilic organisms. The temperature can be kept constant or can be altered during the experiment. The pH of the medium may be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of an acid or base, such as acetic acid, sulfuric acid, phosphoric acid, NaOH, KOH or NH₄OH during growth. If complex medium components such as yeast extract are utilized, the necessity for additional buffers may be reduced, due to the fact that many complex compounds have high buffer capacities. If a fermentor is utilized for culturing the microorganisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml or 250 shake flasks are used, filled with about 10% (by volume) of the required growth medium. The flasks should be shaken on a rotary shaker (amplitude about 25 mm) using a speed-range of about 100-300 'rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD600 of 0.5-1.5 using cells grown on agar plates, such as CM plates (10 g/l glucose, 2.5 g/l NaCl, 2 g/l urea, 10 g/l polypeptone, 5 g/l yeast extract, 5 g/l meat extract, 22 g/l (NH₄)₂SO₄, 2 g/l urea, 10 g/l polypeptone, 5 g/1 yeast extract, 5 g/l meat extract, 22 g/l agar, pH about 6.8 to 7.2 with 2M NaOH) that had been incubated at 30° C. Inoculation of the media is accomplished by either introduction of a saline suspension of C. glutamicum cells from CM plates or addition of a liquid preculture of this bacterium.

### General Methods

Protocols for general methods can be found in Handbook on Corynebacterium glutamicum, (2005) eds.: L. Eggeling, M. Bott., Boca Raton, CRC Press, at Martin et al. (Biotechnology (1987) 5, 137-146), Guerrero et al. (Gene (1994), 138, 35-41), Tsuchiya und Morinaga (Biotechnology (1988), 6, 428-430), Eikmanns et al. (Gene (1991), 102, 93-98), EP 0 472 869, U.S. Pat. No. 4,601,893, Schwarzer and Pühler (Biotechnology (1991), 9, 84-87, Reinscheid et al. (Applied and Environmental Microbiology (1994), 60, 126-132), LaBarre et al. (Journal of Bacteriology (1993), 175, 1001-1007), WO 96/15246, Malumbres et al. (Gene (1993), 134, 15-24), in JP-A-10-229891, at Jensen und Hammer (Biotechnology and Bioengineering (1998), 58, 191-195), Makrides (Microbiological Reviews (1996), 60, 512-538) and in well known textbooks of genetic and molecular biology.

### Strains, Media and Plasmids

Strains can be taken e.g. for example, but not limited to, from the following list:

Corynebacterium glutamicum ATCC 13032,
Corynebacterium acetoglutamicum ATCC 15806,
Corynebacterium acetoacidophilum ATCC 13870,
Corynebacterium thermoaminogenes FERM BP-1539,
Corynebacterium melassecola ATCC 17965,
Brevibacterium flavum ATCC 14067,
Brevibacterium lactofermentum ATCC 13869, and
Brevibacterium divaricatum ATCC 14020 or strains which have been derived therefrom such as Corynebacterium glutamicum KFCC10065, DSM 17322 or
Corynebacterium glutamicum ATCC21608
Corynebacterium efficiens DSMZ44547, 44548, 44549

### Recombinant DNA Technology

Protocols can be found in: Sambrook, J., Fritsch, E. F., and Maniatis, T., in Molecular Cloning: A Laboratory Manual, 3^{rd} edition (2001) Cold Spring Harbor Laboratory Press, NY, Vol. 1, 2, 3, and Handbook on Corynebacterium glutamicum (2005) eds. L. Eggeling, M. Bott., Boca Raton, CRC Press.

### Quantification of Amino Acids and Methionine Intermediates.

The analysis is done by HPLC (Agilent 1100, Agilent, Waldbronn, Germany) with a guard cartridge and a Synergi 4 μm column (MAX-RP 80 Å, 150*4.6 mm) (Phenomenex, Aschaffenburg, Germany). Prior to injection the analytes are derivatized using o-phthaldialdehyde (OPA) and mercaptoethanol as reducing agent (2-MCE). Additionally sulfhydryl groups are blocked with iodoacetic acid. Separation is carried out at a flow rate of 1 ml/min using 40 mM NaH₂PO₄ (eluent A, pH=7.8, adjusted with NaOH) as polar and a methanol water mixture (100/1) as non-polar phase (eluent B). The following gradient is applied: Start 0% B; 39 min 39% B; 70 min 64% B; 100% B for 3.5 min; 2 min 0% B for equilibration.

Derivatization at room temperature is automated as described below. Initially 0.5 μl of 0.5% 2-MCE in bicine (0.5M, pH 8.5) are mixed with 0.5 μl cell extract. Subsequently 1.5 μl of 50 mg/ml iodoacetic acid in bicine (0.5M, pH 8.5) are added, followed by addition of 2.5 μl bicine buffer (0.5M, pH 8.5). Derivatization is done by adding 0.5 μl of 10 mg/ml OPA reagent dissolved in 1/45/54 v/v/v of 2-MCE/MeOH/bicine (0.5M, pH 8.5). Finally the mixture is diluted with 32 μl H₂O. Between each of the above pipetting steps there is a waiting time of 1 min. A total volume of 37.5 μl is then injected onto the column. Note, that the analytical results can be significantly improved, if the auto sampler needle is periodically cleaned during (e.g. within waiting time) and after sample preparation. Detection is performed by a fluorescence detector (340 nm excitation, emission 450 nm, Agilent, Waldbronn, Germany). For quantification α-amino butyric acid (ABA) is used as internal standard

### Definition of Recombination Protocol

In the following it will be described how a strain of C. glutamicum with increased efficiency of methionine production can be constructed implementing the findings of the above predictions. Before the construction of the strain is described, a definition of a recombination event/protocol is given that will be used in the following.

“Campbell in,” as used herein, refers to a transformant of an original host cell in which an entire circular double stranded DNA molecule (for example a plasmid being based on pCLIK int sacB has integrated into a chromosome by a single homologous recombination event (a cross-in event), and that effectively results in the insertion of a linearized version of said circular DNA molecule into a first DNA sequence of the chromosome that is homologous to a first DNA sequence of the said circular DNA molecule. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point. The name comes from Professor Alan Campbell, who first proposed this kind of recombination.

“Campbell out,” as used herein, refers to a cell descending from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated Campbelled in DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter-selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter-selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc. The term “Campbell in” and “Campbell out” can also be used as verbs in various tenses to refer to the method or process described above.

It is understood that the homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

For practicality, in C. glutamicum, typical first and second homologous DNA sequence are at least about 200 base pairs in length, and can be up to several thousand base pairs in length, however, the procedure can be made to work with shorter or longer sequences. For example, a length for the first and second homologous sequences can range from about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs. The “Campbell In and -Out-method” is described in WO2007012078

### EXAMPLES

The following experiments demonstrate how overexpression of C. jeiekeium formate-THF-synthetase and gcvHTP as well as lipB or lpl leads to increased methionine production. These examples are however in no way meant to limit the invention in any way.

### Shake Flask Experiments and HPLC Assay

Shake flasks experiments, with the standard Molasses Medium, were performed with strains in duplicate or quadruplicate. Molasses Medium contained in one liter of medium: 40 g glucose; 60 g molasses; 20 g (NH₄)₂ SO₄; 0.4 g MgSO₄.7H₂O; 0.6 g KH₂PO₄; 10 g yeast extract (DIFCO); 5 ml of 400 mM threonine; 2 mgFeSO₄.7H₂O; 2 mg of MnSO₄.H₂O; and 50 g CaCO₃ (Riedel-de Haen), with the volume made up with ddH₂O. The pH was adjusted to 7.8 with 20% NH₄OH. 20 ml of continuously stirred medium (in order to keep CaCO₃ suspended) was added to 250 ml baffled Bellco shake flasks and the flasks were autoclaved for 20 min. Subsequent to autoclaving, 4 ml of “4B solution” was added per liter of the base medium (or 80 μl/flask). The “4B solution” contained per liter: 0.25 g of thiamine hydrochloride (vitamin B1), 50 mg of cyanocobalamin (vitamin B12), 25 mg biotin, 1.25 g pyridoxine hydrochloride (vitamin B6) and was buffered with 12.5 mM KPO₄, pH 7.0 to dissolve the biotin, and was filter sterilized. Cultures were grown in baffled flasks covered with Bioshield paper secured by rubber bands for about 48 hours at about 28° C. or 30° C. and at 200 or 300 rpm in a New Brunswick Scientific floor shaker. Samples were typically taken at about 24 hours and/or about 48 hours. Cells were removed by centrifugation followed by dilution of the supernatant with an equal volume of 60% acetonitrile or 60% ethanol and then membrane filtration of the solution mixture using Centricon 0.45 μm spin columns. The filtrates were assayed using HPLC for the concentrations of methionine, glycine plus homoserine, O-acetylhomoserine, threonine, isoleucine, lysine, and other indicated amino acids.

For the HPLC assay, filtered supernatants were diluted 1:100 with 0.45 μm filtered 1 mM Na₂EDTA and 1 μl of the solution was derivatized with OPA reagent (AGILENT) in Borate buffer (80 mM NaBO₃, 2.5 mM EDTA, pH 10.2) and injected onto a 200×4.1 mm Hypersil 5μ AA-ODS column run on an Agilent 1100 series HPLC equipped with a G1321A fluorescence detector (AGILENT). The excitation wavelength was 338 nm and the monitored emission wavelength was 425 nm. Amino acid standard solutions were chromatographed and used to determine the retention times and standard peak areas for the various amino acids. Chem Station, the accompanying software package provided by Agilent, was used for instrument control, data acquisition and data manipulation. The hardware was an HP Pentium 4 computer that supports Microsoft Windows NT 4.0 updated with a Microsoft Service Pack (SP6a).

### Experiment 1

### Generation of the M2014 Strain

C. glutamicum strain ATCC 13032 was transformed with DNA A (also referred to as pH273) (SEQ ID NO: 21) and “Campbelled in” to yield a “Campbell in” strain. The “Campbell in” strain was then “Campbelled out” to yield a “Campbell out” strain, M440, which contains a gene encoding a feedback resistant homoserine dehydrogenase enzyme (hom^{fbr}). The resultant homoserine dehydrogenase protein included an amino acid change where S393 was changed to F393 (referred to as Hsdh S393F).

The strain M440 was subsequently transformed with DNA B (also referred to as pH373) (SEQ ID NO: 22) to yield a “Campbell in” strain. The “Campbell in” strain were then “Campbelled out” to yield a “Campbell out” strain, M603, which contains a gene encoding a feedback resistant aspartate kinase enzyme (Ask^{fbr}) (encoded by lysC). In the resulting aspartate kinase protein, T311 was changed to I311 (referred to as LysC T3111).

It was found that the strain M603 produced about 17.4 mM lysine, while the ATCC13032 strain produced no measurable amount of lysine. Additionally, the M603 strain produced about 0.5 mM homoserine, compared to no measurable amount produced by the ATCC13032 strain, as summarized in Table 2.

TABLE 2
Amounts of homoserine, O-acetylhomoserine, methionine and
lysine produced by strains ATCC13032 and M603

| | Homoserine | O-acetyl homoserine | Methionine | Lysine |
|---|---|---|---|---|
| Strain | (mM) | (mM) | (mM) | (mM) |
| ATCC13032 | 0.0 | 0.4 | 0.0 | 0.0 |
| M603 | 0.5 | 0.7 | 0.0 | 17.4 |

The strain M603 was transformed with DNA C (also referred to as pH304) (SEQ ID NO:23) to yield a “Campbell in” strain, which was then “Campbelled out” to yield a “Campbell out” strain, M690. The M690 strain contained a PgroES promoter upstream of the metH gene (referred to as P₄₉₇ metH). The sequence of the P₄₉₇ promoter is depicted in SEQ ID NO: 4. The M690 strain produced about 77.2 mM lysine and about 41.6 mM homoserine, as shown below in Table 3.

TABLE 3
Amounts of homoserine, O-acetyl homoserine, methionine and
lysine produced by the strains M603 and M690

| | Homoserine | | Methionine | Lysine |
|---|---|---|---|---|
| Strain | (mM) | O-acetyl homoserine (mM) | (mM) | (mM) |
| M603 | 0.5 | 0.7 | 0.0 | 17.4 |
| M690 | 41.6 | 0.0 | 0.0 | 77.2 |

The M690 strain was subsequently mutagenized as follows: an overnight culture of M603, grown in BHI medium (BECTON DICKINSON), was washed in 50 mM citrate buffer pH 5.5, treated for 20 min at 30° C. with N-methyl-N-nitrosoguanidine (10 mg/ml in 50 mM citrate pH 5.5). After treatment, the cells were again washed in 50 mM citrate buffer pH 5.5 and plated on a medium containing the following ingredients: (all mentioned amounts are calculated for 500 ml medium) 10 g (NH₄)₂SO₄; 0.5 g KH₂PO₄; 0.5 g K₂HPO₄; 0.125 g MgSO₄.7H₂O; 21 g MOPS; 50 mg CaCl₂; 15 mg protocatechuic acid; 0.5 mg biotin; 1 mg thiamine; and 5 g/l D,L-ethionine (SIGMA CHEMICALS, CATALOG #E5139), adjusted to pH 7.0 with KOH. In addition the medium contained 0.5 ml of a trace metal solution composed of: 10 g/l FeSO_{4*}7H₂O; 1 g/l MnSO₄*H₂O; 0.1 g/l ZnSO₄.7H₂O; 0.02 g/l CuSO₄; and 0.002 g/l NiCl₂*6H₂O, all dissolved in 0.1 M HCl. The final medium was sterilized by filtration and to the medium, 40 mls of sterile 50% glucose solution (40 ml) and sterile agar to a final concentration of 1.5% were added. The final agar containing medium was poured to agar plates and was labeled as minimal-ethionine medium. The mutagenized strains were spread on the plates (minimal-ethionine) and incubated for 3-7 days at 30° C. Clones that grew on the medium were isolated and restreaked on the same minimal-ethionine medium. Several clones were selected for methionine production analysis.

Methionine production was analyzed as follows. Strains were grown on CM-agar medium for two days at 30° C., which contained: 10 g/l D-glucose, 2.5 g/l NaCl; 2 g/l urea; 10 g/l Bacto Peptone (DIFCO); 5 g/l Yeast Extract (DIFCO); 5 g/l Beef Extract (DIFCO); 22 g/l Agar (DIFCO); and which was autoclaved for 20 min at about 121° C.

After the strains were grown, cells were scraped off and resuspended in 0.15 M NaCl. For the main culture, a suspension of scraped cells was added at a starting OD of 600 nm to about 1.5 to 10 ml of Medium II (see below) together with 0.5 g solid and autoclaved CaCO₃ (RIEDEL DE HAEN) and the cells were incubated in a 100 ml shake flask without baffles for 72 h on a orbital shaking platform at about 200 rpm at 30° C. Medium II contained: 40 g/l sucrose; 60 g/l total sugar from molasses (calculated for the sugar content); 10 g/l (NH₄)₂SO₄; 0.4 g/l MgSO₄*7H₂O; 0.6 g/l KH₂PO₄; 0.3 mg/l thiamine*HCl; 1 mg/l biotin; 2 mg/l FeSO₄; and 2 mg/l MnSO₄. The medium was adjusted to pH 7.8 with NH₄OH and autoclaved at about 121° C. for about 20 min). After autoclaving and cooling, vitamin B₁₂ (cyanocobalamine) (SIGMA CHEMICALS) was added from a filter sterile stock solution (200 μg/ml) to a final concentration of 100 μg/l.

Samples were taken from the medium and assayed for amino acid content. Amino acids produced, including methionine, were determined using the Agilent amino acid method on an Agilent 1100 Series LC System HPLC. (AGILENT). A pre-column derivatization of the sample with ortho-pthalaldehyde allowed the quantification of produced amino acids after separation on a Hypersil AA-column (AGILENT).

Clones that showed a methionine titer that was at least twice that in M690 were isolated. One such clone, used in further experiments, was named M1197 and was deposited on May 18, 2005, at the DSMZ strain collection as strain number DSM 17322. Amino acid production by this strain was compared to that by the strain M690, as summarized below in Table 4.

TABLE 4
Amounts of homoserine, O-acetylhomoserine, methionine and
lysine produced by strains M690 and M1197

| | Homoserine | O-acetyl- | Methionine | Lysine |
|---|---|---|---|---|
| Strain | (mM) | homoserine (mM) | (mM) | (mM) |
| M690 | 41.6 | 0.0 | 0.0 | 77.2 |
| M1179 | 26.4 | 1.9 | 0.7 | 79.2 |

The strain M1197 was transformed with DNA F (also referred to as pH399, SEQ ID NO: 24) to yield a “Campbell in” strain, which was subsequently “Campbelled out” to yield strain M1494. This strain contains a mutation in the gene for the homoserine kinase, which results in an amino acid change in the resulting homoserine kinase enzyme from T190 to A190 (referred to as HskT190A). Amino acid production by the strain M1494 was compared to the production by strain M1197, as summarized below in Table 5.

TABLE 5
Amounts of homoserine, O-acetylhomoserine, methionine and
lysine produced by strains M1197 and M1494

| | Homoserine | O-acetyl- | Methionine | Lysine |
|---|---|---|---|---|
| Strain | (mM) | homoserine (mM) | (mM) | (mM) |
| M1197 | 26.4 | 1.9 | 0.7 | 79.2 |
| M1494 | 18.3 | 0.2 | 2.5 | 50.1 |

The strain M1494 was transformed with DNA D (also referred to as pH484, SEQ ID NO:25) to yield a “Campbell in” strain, which was subsequently “Campbelled out” to yield the M1990 strain. The M1990 strain overexpresses a metY allele using both a groES-promoter and an EFTu (elongation factor Tu)-promoter (referred to as P₄₉₇ P₁₂₈₄ metY). The sequence of P₄₉₇P₁₂₈₄ promoter is set forth in SEQ ID NO:26 Amino acid production by the strain M1494 was compared to the production by strain M1990, as summarized below in Table 6.

TABLE 6
Amounts of homoserine, O-acetylhomoserine, methionine and
lysine produced by strains M1494 and M1990

| | Homoserine | O-acetyl- | Methionine | Lysine |
|---|---|---|---|---|
| Strain | (mM) | homoserine (mM) | (mM) | (mM) |
| M1494 | 18.3 | 0.2 | 2.5 | 50.1 |
| M1990 | 18.2 | 0.3 | 5.6 | 48.9 |

The strain M1990 was transformed with DNA E (also referred to as pH 491, SEQ ID NO: 27) to yield a “Campbell in” strain, which was then “Campbelled out” to yield a “Campbell out” strain M2014. The M2014 strain overexpresses a metA allele using a superoxide dismutase promoter (referred to as P₃₁₁₉ metA). The sequence of P₃₁₁₉ promoter is set forth in SEQ ID NO: 3. Amino acid production by the strain M2014 was compared to the production by strain M1990, as summarized below in Table 7

TABLE 7
Amounts of homoserine, O-acetylhomoserine, methionine and
lysine produced by strains M1494 and M1990

| | Homoserine | O-acetyl- | Methionine | Lysine |
|---|---|---|---|---|
| Strain | (mM) | homoserine (mM) | (mM) | (mM) |
| M1990 | 18.2 | 0.3 | 5.6 | 48.9 |
| M2014 | 12.3 | 1.2 | 5.7 | 49.2 |

### Experiment 2

### Deletion of mcbR from M2014

Plasmid pH429 containing an RXA00655 deletion, (SEQ ID NO:28) was used to introduce the mcbR deletion into C. glutamicum via integration and excision (see WO 2004/050694 A1).

Plasmid pH429 was transformed into the M2014 strain with selection for kanamycin resistance (Campbell in). Using sacB counter-selection, kanamycin-sensitive derivatives of the transformed strain were isolated which presumably had lost the integrated plasmid by excision (Campbell out). The transformed strain produced kanamycin-sensitive derivatives that made small colonies and larger colonies. Colonies of both sizes were screened by PCR to detect the presence of mcbR deletion. None of the larger colonies contained the deletion, whereas 60-70% of the smaller colonies contained the expected mcbR deletion.

When an original isolate was streaked for single colonies on BHI plates, a mixture of tiny and small colonies appeared. When the tiny colonies were restreaked on BHI, once again a mixture of tiny and small colonies appeared. When the small colonies were restreaked on BHI, the colony size was usually small and uniform. Two small single colony isolates, called OM403-4 and OM403-8, were selected for further study.

Shake flask experiments (Table 8) showed that OM403-8 produced at least twice the amount of methionine as the parent M2014. This strain also produced less than one-fifth the amount of lysine as M2014, suggesting a diversion of the carbon flux from aspartate semialdehyde towards homoserine. A third striking difference was a greater than 10-fold increase in the accumulation of isoleucine by OM403 relative to M2014. Cultures were grown for 48 hours in standard molasses medium.

TABLE 8
Amino acid production by isolates of the OM403 strain in shake flask
cultures inoculated with freshly grown cells

| | | Deletion | Met | Lys | Hse + Gly | Ile |
|---|---|---|---|---|---|---|
| Strain | Colony size | ΔmcbR | (g/l) | (g/l) | (g/l) | (g/l) |
| M2014 | Large | none | 0.2 | 2.4 | 0.3 | 0.04 |
| | | | 0.2 | 2.5 | 0.3 | 0.03 |
| | | | 0.2 | 2.4 | 0.3 | 0.03 |
| | | | 0.4 | 3.1 | 0.4 | 0.03 |
| OM403-8 | Small | ΔRXA0655 | 1.0 | 0.3 | 0.8 | 0.8 |
| | | | 1.0 | 0.3 | 0.8 | 0.8 |
| | | | 0.9 | 0.3 | 0.8 | 0.8 |
| | | | 1.0 | 0.3 | 0.8 | 0.6 |

Also as shown in Table 9, there was a greater than 15-fold decrease in the accumulation of O-acetylhomoserine by OM403 relative to M2014. The most likely explanation for this result is that most of the O-acetylhomoserine that accumulates in M2014 is being converted to methionine, homocysteine, and isoleucine in OM403. Cultures were grown for 48 hours in standard molasses medium.

TABLE 9
Amino acid production by two isolates of OM403 in shake flask
cultures inoculated with freshly grown cells.

| | Deletion | Met | OAc-Hse | Ile |
|---|---|---|---|---|
| Strain | ΔmcbR | (g/l) | (g/l) | (g/l) |
| M2014 | None | 0.4 | 3.4 | 0.1 |
| | | 0.4 | 3.2 | 0.1 |
| OM403-4 | ΔRXA0655 | 1.7 | 0.2 | 0.3 |
| | | 1.5 | 0.1 | 0.3 |
| OM403-8 | ΔRXA0655 | 2.2 | <0.05 | 0.6 |
| | | 2.5 | <0.05 | 0.6 |

### Experiment 3

### Decreasing metQ Expression

In order to decrease the import of methionine in OM403-8, the promoter and 5′ portion of the metQ gene were deleted. The metQ gene encodes a subunit of a methionine import complex that is required for the complex to function. This was accomplished using the standard Campbelling in and Campbelling out technique with plasmid pH449 (SEQ ID NO: 29). OM403-8 and OM456-2 were assayed for methionine production in shake flask assays. The results (Table 10) show that OM456-2 produced more methionine than OM403-8. Cultures were grown for 48 hours in standard molasses medium.

TABLE 10
Shake flask assays of OM456-2

| | | [Met] | [Lys] | [Gly/Hse] | [OAcHS] | [Ile] |
|---|---|---|---|---|---|---|
| Strain | vector | (g/l) | (g/l) | (g/l) | (g/l) | (g/l) |
| OM403-8 | none | 4.0 | 0.8 | 2.2 | 0.4 | 1.9 |
| | | 3.9 | 0.6 | 2.2 | 0.4 | 1.9 |
| OM456-2 | none | 4.2 | 0.4 | 2.3 | 0.4 | 2.3 |
| | | 4.3 | 0.5 | 2.4 | 0.4 | 2.3 |

### Experiment 4

### Construction of OM469

A strain referred to as OM469 was constructed which included both deletion of metQ and overexpression of metF by replacing the metF promoter with the phage λP_{R} promoter in OM456-2. This was accomplished using the standard Campbelling in and Campbelling out technique with plasmid pOM427 (SEQ ID NO 30). Four isolates of OM469 were assayed for methionine production in shake flask culture assays where they all produced more methionine than OM456-2, as shown in Table 11. Cultures were grown for 48 hours in standard molasses medium containing 2 mM threonine.

TABLE 11
Shake flask assays of OM469, a derivative of OM456-2 containing the
phage lambda P_{R} promoter in place of the metF promoter.

| | | metF | | | | [Gly/ | | |
|---|---|---|---|---|---|---|---|---|
| | | pro- | | [Met] | [Lys] | Hse] | [OAcHS] | [Ile] |
| Strain | | moter | MetQ | (g/l) | (g/l) | (g/l) | (g/l) | (g/l) |
| OM428-2 | | λP_{R} | native | 4.5 | 0.5 | 2.6 | 0.4 | 2.6 |
| | | | | 4.6 | 0.4 | 2.6 | 0.3 | 2.5 |
| OM456-2 | | native | ΔmetQ | 4.2 | 0.4 | 2.4 | 0.3 | 2.5 |
| | | | | 4.2 | 0.5 | 2.4 | 0.3 | 2.5 |
| OM469 | -1 | λP_{R} | ΔmetQ | 5.0 | 0.5 | 2.7 | 0.4 | 3.1 |
| | -2 | | | 4.9 | 0.5 | 2.7 | 0.4 | 2.8 |
| | -3 | | | 4.8 | 0.4 | 2.6 | 0.4 | 2.7 |
| | -4 | | | 4.7 | 0.5 | 2.6 | 0.4 | 2.8 |

### Experiment 5

### Construction of M 2543

The strain OM469-2 was transformed by electroporation with the plasmid pCLIK5A P_{SOD} TKT as depicted in SEQ ID NO. 31. This was accomplished using the standard Campbelling in and Campbelling out technique.

Isolates of OM 469 P_{SOD} TKT which are labelled M2543 were assayed for methionine production in shake flask culture assays, where they produced more methionine than OM469-2. The results of strain M2543 Are shown in Table 12.

TABLE 12
Shake flask assays of OM469 and M2543

| | | met | | | | | | |
|---|---|---|---|---|---|---|---|---|
| | | genes | | | | | | |
| | plas- | on | [Met] | [Lys] | [Gly] | [Hse] | [AHs] | [Ile] |
| Strain | mid | plasmid | (mm | (mm) | (mm) | (mm) | (mm) | (mm) |
| OM469-2 | | None | 14 | 3.4 | 16 | 1.7 | 0.3 | 11.8 |
| M2543# | | None | 20.4 | 1.9 | 21.8 | 0.8 | <0.1 | 12.4 |

### Experiment 6

### Construction of GK1259

In order to decrease expression of serine deaminase (sda), a portion of the sda gene was deleted. This was accomplished using the standard Campbelling in and Campbelling out technique with plasmid pH626 int SacB delta sdaA (SEQ ID No. 32). To this end, strain M2543 was transformed by electroporation with the plasmid pH626 int SacB delta sdaA. The resulting strain was named GK1259.

### Experiment 7

### Construction of OM264C

Plasmid pOM253 (SEQ ID No. 33) was used to delete serA and substitute it with spectinomycin resistance in C. glutamicum strain M2014. The resulting “Campbelled out” strain, M2014 ΔserA::spec, was named OM264C. OM264C is a serine auxotroph. Since it also lacks a functional GCS, OM264C cannot grow on a minimal medium lacking serine but containing glycine. If, however, a functional GCS system is installed in OM264C, then it will gain the ability to grow on minimal medium containing glycine.

The recipe for the minimal (chemically defined) plates was as follows:

| | Concentration | | Volume of | |
|---|---|---|---|---|
| Stock solution name | of Stock | | stock for 1 liter | |
| 10 X Spizizen's Salts | See below | | 100 | ml |
| Glucose | 50% | w/v | 10 | ml |
| 4B's Solution | See below | | 4 | ml |
| Threonine | 400 | mM | 5 | ml |
| Cysteine HCl | 4 | g/l | 10 | ml |
| CaCl₂•2H₂O | 5% | w/v | 5 | ml |
| Sodium citrate | 1.0 | M | 20 | ml |
| Thymidine | 1% | w/v | 10 | ml |
| Phenylalanine | 1% | w/v | 10 | ml |
| Isoleucine | 1% | w/v | 10 | ml |
| Thiamine HCl | 0.1% | w/v | 5 | ml |
| Methionine | 1% | w/v | 5 | ml |
| Sodium succinate | 1.0 | M | 3 | ml |
| Potassium acetate | 5.0 | M | 1.2 | ml |
| Glycine (when added) | 10% | w/v | 5 | ml |
| Serine (when added) | 10% | w/v | 2 | ml |

| | | | |
|---|---|---|---|
| Lipoic acid | 1 g/l in 20 mM | 1 | ml |
| (when added) | potassium phosphate, | | |
| | pH 7.0 | | |

All stocks were filter sterilized. For agar Petri plates, 15 g agar were suspended in 800 ml distilled water and autoclaved.

10× Spizizen's salts
20 g Ammonium sulfate
174 g Potassium phosphate dibasic (trihydrate)
60 g Potassium phosphate monobasic (anhydrous)
10 g Sodium citrate (dihydrate)
2 g Magnesium sulfate (heptahydrate)
Distilled water to one liter
Add 3.5 ml 0.4% FeCl₃.6H₂O filter sterilized and
1 ml Micronutrient solution (see below). Final pH should be about 7.2.
Filter sterilize.
Micronutrient solution: amount for 1 liter

### 0.15 g Na₂MoO₄.2H₂O

### 2.5 g H₃BO3

### 0.7 g CoCl₂.6H₂O

### 0.25 g CuSO₄.5H₂O

### 1.6 g MnCl₂.4H₂O

### 0.3 g ZnSO₄.7H₂O

Distilled water to one liter.
Filter sterilize.
4B's solution: amount for one liter

### 0.25 g Thiamine HCl (Vitamin B₁)

### 50 mg Cyanocobalamin (Vitamin B₁₂)

### 25 to 28 mg Biotin

### 1.25 g Pyridoxine HCl (Vitamin B₆)

Dissolve in 50 mM potassium phosphate, pH 7.0
Filter sterilize. Store in the dark.

### Experiment 8

### Construction of Strains Expressing the C. jeikeium gcvPTH Genes in C. glutamicum.

Unlike C. glutamicum, a close relative named C. jeikeium does contain a GCS. In the C. jeikeium chromosome, the gcvP, T, and H genes are clustered together in an operon (Tauch et al., 2005, J. Bacteriol., vol 187, pp 4671-4682). This cluster was cloned in four overlapping subset pieces by polymerase chain reaction (PCR) using C. jeikeium strain K411 chromosomal DNA as the template.

The necessary DNA was obtained by dividing the sequence into four smaller regions and obtaining four independent smaller PCR fragments. The four pieces were amplified with four sets of primers. An artificial XbaI site and an artificial ribosome binding site were engineered just upstream from the gcvP start codon in the respective sense primer, and an artificial BamHI site was engineered just downstream from the gcvH stop codon in the respective antisense primer. This allowed the coding sequences of the gcvPTH cluster to be reconstituted and carried on an XbaI to BamHI fragment.

The resulting XbaI to BamHI fragment containing a reconstituted gcvPTH cluster was next cloned into C. glutamicum replicating plasmids designed to express genes from the C. glutamicum groESL promoter, herein named P₄₉₇, or a B. subtilis phage SPO1 promoter, herein named P₁₅ (SEQ ID No. 42). The resulting plasmids were named pOM615 (SEQ ID No. 34) and pOM616 (SEQ ID No. 35), respectively. The P₄₉₇ and P₁₅ promoters were used because they promote constitutive expression of genes situated downstream. In particular, these promoters are not significantly regulated by any glycine related metabolite, such as glycine, serine, methionine, thymidine, purine etc.

### Experiment 9

### The C. jeikeium gcvPTH Genes Function in C. glutamicum

Plasmids pOM615, pOM616, and empty vector pCLIK were each separately transformed into the tester strain C. glutamicum strain OM264C and the methionine producer C. glutamicum strain GK1259 using selection on Brain Heart Infusion (formerly Difco, now Becton Dickenson) agar plates containing kanamycin sulfate (25 mg/l). The OM264C transformants were streaked on minimal plates lacking serine but containing glycine. Lipoic acid was added to the medium to give a final concentration of 1 mg/l to ensure that a sufficient amounts of this cofactor of GcvH was present.

Both, the pOM615 and pOM616 derived strains, OM264C(pOM615) and OM264C(pOM616) as well as GK1259(pOM615) and GK1259(pOM616) grew, while the pCLIK transformant (OM264C(pCLIK) did not.

### Experiment 10

### The lipBA Genes from C. jeikeium Function in C. glutamicum.

C. glutamicum is a lipoic acid prototroph, and C. glutamicum presumably has a lipoyl synthetase, since pyruvate dehydrogenase and α-ketoglutarate dehydrogenase are active.

In E. coli, there are two different pathways for attachment (ligation) of lipoic acid to target proteins, the LipB dependent pathway for endogenously synthesized lipoic acid, and the LplA pathway for fed lipoic acid (Morris et al., 1995, J. Bacteriol. Vol 177, pp 1-10).

By sequence comparison, C. glutamicum seems to have good homologs for both LipB and LplA. Thus, the lipB gene and its native promoter, together with the lipA gene, was cloned by PCR from C. jeikeium, strain K411 chromosomal DNA as a template. The resulting blunt PCR fragment was ligated into the unique SwaI site of pOM615 or pOM616 to give pOM620AF (SEQ ID No 36) and pOM621AR (SEQ ID No 37), respectively.

Plasmids pOM620AF and pOM621AR were each transformed into strain OM264C resulting in OM264C(pOM620AF) and OM264C(pOM621AR) respectively and into methionine producing strain GK1259 resulting in GK1259(pOM620AF) and GK1259(pOM621AR), respectively. The OM264C transformants were streaked on the minimal glycine plates described above (but without lipoic acid), and both transformants grew, while OM264C(pCLIK) did not, demonstrating that the C. jeikeium LipB pathway could lipoylate the C. jeikeium GcvH protein when the two were expressed together in C. glutamicum.

The GK1259 transformants of pOM615, pOM616, pOM620AF, pOM621AR, and pCLIK were tested for methionine and glycine production in shake flasks using molasses medium, without lipoic acid or with lipoic acid added to a final concentration of about 10 mg/ml. The results are shown in Tables 13 and 14 below.

TABLE 13
Methionine and glycine production by GK1259 transformed
with various plasmids designed to express C. jeikeium gcvPTH
with lipBA and grown in shake flasks in molasses medium.

| | Promoter | | Lipoic | Glycine | Methionine |
|---|---|---|---|---|---|
| Plasmid | for gcvPTH | lipBA | acid | g/l | g/l |
| pCLIK | − | − | − | 2.5 | 4.3 |
| pOM615 | P₄₉₇ | − | + | 0.1 | 4.7 |
| pOM620AF | P₄₉₇ | + | − | 0.0 | 5.0 |

TABLE 14
Methionine and glycine production by O264C transformed
with various plasmids designed to express C. jeikeium gcvPTH
with lipBA and grown in shake flasks in molasses medium.

| | Promoter | | | | |
|---|---|---|---|---|---|
| | for | | added | Glycine* | Methionine* |
| Plasmid | gcvPTH | lipBA | lipoic acid | g/l | g/l |
| pCLIK | − | − | − | 2.1 | 3.8 |
| pOM616 | P₁₅ | − | + | 0.0 | 4.1 |
| POM621AR | P₁₅ | + | − | 0.0 | 3.9 |

*Methionine and glycine titers are averages of duplicate samples, except for the pOM616 plus lipoic acid sample, which is a single sample.

From these results, it is clear that the excess glycine by-product is consumed and the methionine titer is improved by expressing the C. jeikeium gcvPTH operon. Further improvement is achieved by expressing the C. jeikeium gcvPTH operon and the C. jeikeium lipBA operon from the same plasmid.

### Experiment 11

### Expression of gcvPTH and lipBA from Integrated Cassettes

In the examples given above, the gcv and lip genes were installed on plasmids that replicate in the C. glutamicum. However, these genes can also be installed through an integrating vector. For example, the C. jeikeium gcvPTH operon expressed from promoter P₁₅ has been ligated into an integrating vector to give pOM627 (SEQ ID No. 38), which is designed to integrate at the bioB locus of C. glutamicum.

pOM627 can be “Campbelled in” and “Campbelled out” of strains OM469 and GK1259. As in the above example, one observes improved methionine production and reduced glycine accumulation as is shown in Table 15.

TABLE 15
Methionine and glycine production by transformants of
OM469 and GK1259 using pOM627 designed to
express C. jeikeium gcvPTH from P₁₅ after
integration at bioB, and grown in shake flasks in molasses
medium with or without 10 mg/l lipoic acid added.

| | Lipoic | Glycine | Methionine |
|---|---|---|---|
| Strain | acid | g/l | g/l |
| OM469(pOM627)K | − | 2.7 | 3.8 |
| OM469(pOM627)K | + | 2.0 | 4.0 |
| GK1259(pOM627)K | − | 2.8 | 3.9 |
| GK1259(pOM627)K | + | 2.0 | 4.1 |

The effect from the integrating plasmid pOM627 on glycine and methionine production can be improved by installing multiple copies or by increasing the promoter strength.

The C. jeikeium lipBA operon can also be added (either on replicating or integrating vectors). An example of an integrating plasmid that expresses lipBA from promoter P₄₉₇ after integrating at the bioAD locus of C. glutamicum is pOM180 (SEQ ID No 39).

### Experiment 12

### The E. coli GCS System Also Functions in C. glutamicum

The E. coli lpd gene was amplified by PCR and installed in an integrating plasmid to give pOM331 (SEQ ID No 40). The integration site is a gene herein named metE2, a gene that is homologous to a portion of metE, but which does not appear to be essential for growth or methionine production in C. glutamicum.

In pOM331, the E. coli lpd gene is expressed from the P₄₉₇ promoter. pOM331 was transformed into and Campbelled out of OM264C to give new strain OM197, which was confirmed by an appropriate diagnostic PCR to contain the integrated P₄₉₇ lpd cassette. In addition, the E. coli gcvTHP operon was amplified by PCR and ligated just downstream from the P₄₉₇ promoter in a replicating vector to give pOM344 (SEQ ID No. 41). pOM344 and pCLIK were each separately transformed into strain OM197, and the resulting strains were streaked on minimal glycine plates containing glycine and lipoic acid, but lacking serine (see above).

After several days at 30° C., OM197/pOM344 had grown, but OM197/pCLIK had not, demonstrating that the E. coli GCS system was functioning in C. glutamicum.

In the above examples, it is shown that genes encoding GCS subunits P, T and H, and genes encoding enzymes that catalyze lipoic acid ligation or synthesis, cloned from either of two “donor” organisms (which are relatively unrelated to each other) are each capable of functioning to give measurable GCS activity in C. glutamicum, either by showing complementation of a serine auxotrophy on plates containing glycine but no serine or by showing a decrease in glycine production by a methionine producing strain compared to the glycine production of the relevant parent or precursor strain transformed with an empty vector as a control.

By extension, it seems reasonable to assume that one skilled in the art will be able to follow the examples disclosed herein and reconstitute other GCS systems in C. glutamicum and that GCS activity can be established and/or increased in other microorganisms by cloning the relevant genes from the same (i.e. E. coli and C. jeikeium) or other donor organisms. An improvement may also be achieved by feeding lipoic acid e.g. at about 0.1 to 10 mg/l.

### Experiment 13

### Construction of M2616

C. glutamicum M2616 was constructed. This strain which shows deleted serA activity allows for testing formate THF synthetase function.

Plasmid pHF96 (SEQ ID 43) is an integrating plasmid designed to create a deletion-substitution in the serA gene of C. glutamicum strains related to C. glutamicum ATCC 13032. Plasmid pHF96 int sacB delta serA was used to delete sera in M2543. The plasmid was transformed in to C. glutamicum M2543 cby electroporation and kanamycin resistant clones were isolated. After determining these kanamycin-resistant as successful “Campbelled in” strains by PCR the strains grown overnight in liquid CM medium and were plated on sucrose (10% concentration) containing CM Medium. The resulting “Campbelled out” strain, M2543 delta serA, was named M2616. As expected, M2616 is a serine auxotroph when assayed on minimal medium. Also as expected, since it lacks a functional formate THF synthetase, M2616 cannot grow on a minimal medium lacking serine but containing glycine and formate (see Example for recipe for minimal (chemically defined) medium). If a functional formate THF synthetase system is installed in M2616, then it will gain the ability to grow on minimal medium containing glycine.

### Experiment 14

### Cloning of Two Formyl-THF Synthetase Genes from Two Sources of Corynebacterium jeikeium

Unlike C. glutamicum, a close relative named Corynebacterium jeikeium does contain a formyl-THF synthetase protein with the accession number NP_{—}939608 and a corresponding gene with the accession number GeneID: 2649808.

Two sources of templates were utilized for the cloning of the formyl-THF synthetase from Corynebacterium jeikeium. DNA derived from the strain of the NCTC National Collection of Type Cultures London, UK, number 11915 with the strain designation K411 and from the DSMZ strain 7171 were used. Chromosomal DNA was prepared using the Quiagen DNA Kit as described by the manufacturer. Oligonucleotides HS1304 (SEQ ID No: 44) and HS1305 (SEQ ID No. 45) were used to amplify the genomic sequences of the formate-THF synthetase gene from the two sources of genomic DNA (NCTC 11915 and DZMZ 7171). Pwo polymerase from Roche Mannheim was used at the following conditions: Annealing at 52° C. for 30″ and elongation at 72° C. for 120″ yielded a PCR fragment of about 1700 Bp.

In addition primers HS1302 (SEQ ID No. 46) and HS1303 (SEQ ID No. 47) were used to amplify the promotor expression unit with the sequence as described from chromosomal DNA derived from the strain ATCC13032. Pwo polymerase from Roche Mannheim was used at the following conditions: Annealing at 53° C. for 30″ and elongation at 72° C. for 30″ yielded a PCR fragment of about 200 Bp.

Both fragments were added in a third PCR in which the primers HS13032 and HS1305 were added. The third PCR was performed with limiting amounts of the PCR fragments I and II at sufficient amounts of the end to end primers and was a fused using the end- to end primers. HS1302+HS1305 were used to amplify a fusion construct of the PCR fragments resulting from primers HS1302+HS1303 and HS1304+HS1305. Pwo polymerase from Roche Mannheim was used at the following conditions: Annealing at 55° C. for 30″ and elongation at 72° C. for 120″ yielded a PCR fragment of about 1900 Bp.

The fragment was purified with the GFX PCR purification kit and was digested using the restriction enzymes MluI and XbaI. Positive plasmids containing the insert of the formyl-THF synthetase gene were sequenced.

The gene derived from C. jeikeium NCTC K11915 was sequenced. Sequencing of this gene revealed the sequence to be as expected. The gene derived from C. jeikeium DSMZ 7171 was sequenced. Sequencing of this gene revealed a sequence as described in the plasmid sequence pH657.

Plasmid pH655 (SEQ ID No. 48) comprising formate-THF-synthetase from NCTC K11915 and pH657 (SEQ ID No. 49) comprising formate-THF-synthetase from DSMZ7171 were transformed into the strain M2616 lacking the functional serA gene by electroporation.

The resulting strains M2616(pH655) and M2616(pH657) as well as the strain lacking a plasmid were streaked on minimal medium containing 10 mM threonine, 20 mM Na-formate and 20 mM glycine+−10 mM serine. Strains containing the plasmids pH655 and pH657 but not pCLIK5a formed colonies on minimal medium containing formate and glycine, while even after prolonged incubation the strain M2616 did not produce colonies on this medium lacking serine. On the same minimal medium with added threonine, Na-formate and glycine but with added serine (20 mM) all strains formed colonies including M2616.

This result showed the successful functional expression of the formate THF synthetase derived from Corynebacterium jeikeium NCTC and DSMZ 7171 in Corynebacterium glutamicum to provide a strain which utilizes formate in the synthesis of serine.

### Experiment 15

### Construction of a Methionine Overproducing Strain which Expresses Formate-THF Synthetase Genes

Plasmids pH655 and pH657 were transformed into the strain GK1259 by electroporation. The resulting strains GK1259(pH655) and GK1259(pH657) as well as the strain containing no plasmid were incubated in shake flask assays as previously described. In addition 20 mM formate was added to the growth medium. It was observed that expression of the formate THF synthetase gene improved the methionine productivity of the strain over the strain lacking the formate THF synthetase gene. The data is found in table 16.

TABLE 16
Shake flask assays of GK1259, and GK1259(pH655)
overexpressing formate-THF synthetase

| | | Gene | [Met] |
|---|---|---|---|
| Strain | Plasmid | overexpressed | (mM/l) |
| GK1259 | None | none | 23.3 |
| GK1259 | pH655 | Formate THF | 24.6 |
| | | synthetase | |

### Experiment 16

### Deletion of Formyl-THF-Deformylase in Strain M2543

It was detected by sequence comparisons that C. glutamicum contains a gene, which has been annotated as a formyl-THF deformylase (Accession Ncg10371) This gene has been annotated as coding for an enzyme with the enzymatic activity of formyl-THF deformylase, cleaving formate from the metabolite formyl-tetrahydrofolate (Annual review of plant physiology and plant molecular biology (2001) 52: 119-137).

Knockout of the formyl-THF deformylase (accession number Ncg 10371) was performed by cloning a DNA fragment in which the upstream region of the gene coding for Ncg10371 (approximately 500 Bp long) and its downstream region (approximately 500 Bp long) of the same gene were amplified, fused by fusion-PCR and cloned into a vector resulting in pH670 int sacB delta deformylase (SEQ ID No. 50). The plasmid is transformed into the strain M2543 to yield first recombinants (“Campbell in step”). After successful screening of correct first recombinants by PCR the strain is grown overnight in liquid culture and is plated on growth medium that contains 10% sucrose. This treatment (“Campbell-out-step”) leads to a strain called GK1546 in which the kanamycin resistance marker and the levan sucrose gene encoded on the plasmid pH670 are successfully crossed-out of the chromosome and subsequently lost from the chromosome and the cell. The successive strains from the Campbell out step are identified as deletions of the formyl-THF deformylase by PCR screening utilizing primers which code for sequences within the 5′ and 3′ regions of the described formyl-THF deformylase. Positive clones show a PCR Fragment, which is approximately 900 bp shorter than the PCR product from a formyl-THF deformylase wildtype strain. The resulting strain was named GK1546.

### Experiment 17

### Construction of a Strain Deleted for Formyl-THF-Deformylase and Overexpressing Formate-THF-Synthetase

The strain GK1546 is transformed with the plasmids pH655 and pH657. Resulting strains GK1546(pH655) and GK1546(pH657) show significantly improved growth behaviour when they are grown on a minimal medium containing formate, glycine and threonine but no serine over the strain M2543.

### Experiment 18

### Expression of Functional Formate-THF-Synthetase for the Production of Methionine

Strain M2616 was transformed with pH655 (formate-THF-synthetase NCTC 11915) and pH657 (formate-THF-synthetase DSMZ 7171). The resulting strains M2616(pH655) and M2616(pH657) were analyzed in shake flask assays as described previously.

The medium was supplied with 20 mM glycine as well as 20 mM Na-formate in addition to the normal composition described above. Shake flasks were incubated at 30° C. with a shaking rate of 200 RPM. After 48 h methionine was determined. It was found that the parental strain M2616 without the formate-THF synthetase expressing plasmid did not grow to measurable OD and it did not utilize the given carbon source. Supernatants were assayed for formate in the supernatant by HPLC. Strains containing the plasmids pH655 and pH657 show an utilisation of all formate added to the medium. In addition it is observed that strains grown on formate, glycine and that expressing the formate THF-synthase gene produced measurable amounts of methionine while the strain M2616 did not produce methionine at all. The results are shown in table 17.

TABLE 17
methionine production in M2616 and
plasmid transformants of M2616

| | | Gene | [Met] |
|---|---|---|---|
| Strain | plasmid | overexpressed | (mM/l) |
| M2616 | None | none | 0 |
| M2616 | pH655 | formate-THF- | 4.1 |
| | | synthetase NCTC | |
| M2616 | pH657 | formate THF- | 5.5 |
| | | synthetase DSMZ | |

In another experiment 20 mM serine was added to the culture medium described in Experiment 18. M2616 and M2616 expressing the formate-THF-synthetase DSMZ 7171 gene were grown in the presence of serine and assayed for methionine produced. The resulting methionine titers in the case of overexpression of the were found to be higher in the case of the strains M2616, that expressed the formate-THF-synthetase DSMZ gene, compared to the strain which does not contain a formate-THF-synthetase gene. The data is found in table 18.

TABLE 18
overproduction of methionine in strains overexpressing
formate-THF synthetase

| | Serine | Gene | [Met] |
|---|---|---|---|
| Strain | added | overexpressed | (mM/l) |
| M2616 | + | none | 6.6 |
| M2616 | + | formate-THF- | 8.9 |
| pH657 | | synthetase DSMZ | |

1-45. (canceled)

46. A microorganism,
wherein said microorganism is derived by genetic modification from a starting microorganism such that said microorganism produces more N⁵,N¹⁰-methylene-THF compared to the starting organism.

47. The microorganism according to claim 46,
wherein the microorganism is selected from the group comprising microorganisms of the genera Enterobacteria, Escherichia, Klebsiella, Corynebacterium, Bacillus, Saccharomyces, Schizosaccharomyces, Pichia, Kluyveromyces, Ashbya, Aspergillus, Brevibacterium and Streptomyces.

48. The microorganism according to claim 47,
wherein the microorganism is preferably selected from the group comprising the species Corynebacterium glutamicum, Corynebacterium acetoglutamicum, Corynebacterium acetoacidophilum, Corynebacterium thermoaminogenes, Corynebacterium jeiekium, Corynebacterium melassecola and Corynebacterium effiziens.

49. The microorganism according to claim 48,
wherein the microorganism is derived from a strain of C. glutamicum.

50. The microorganism according to claim 46,
wherein the microorganism is derived by genetic modification from a starting organism such that the amount and/or activity of formate-THF-synthetase is increased in said microorganism compared to the starting organism.

51. The microorganism according to claim 50,
wherein the microorganism is derived by genetic modification from a starting organism such that the amount and/or activity of formyl-THF-deformylase is decreased in said microorganism compared to the starting organism.

52. The microorganism according to claim 50,
wherein the microorganism is derived by genetic modification from a starting organism such that the amount and/or activity of N⁵,N¹⁰-methenyl-THF-cyclosynthetase, N⁵,N¹⁰-methenyl-THF-reductase and/or N⁵,N¹⁰-methylene-THF-reductase is increased in said microorganism compared to the starting organism.

53. The microorganism according to claim 46,
wherein the enzymatic activity of a glycine cleavage system (GCS) is increased in said microorganism compared to the starting organism.

54. The microorganism according to claim 53,
wherein the amount and/or activity of gcvP, gcvT and gcvH are increased in said microorganism compared to the starting organism.

55. The microorganism according to claim 53,
wherein the amount and/or activity of lipA, lipB or lipA and lipB is increased in said microorganism compared to the starting organism.

56. The microorganism according to claim 53,
wherein the amount and/or activity of lplA is increased in said microorganism compared to the starting organism.

57. The microorganism according to claim 53,
wherein the amount and/or activity of lpd is increased in said microorganism compared to the starting organism.

58. The microorganism according to claim 53,
wherein the coding sequences for gcvP, gcvT, gcvH, IplA, lipA and lipB are derived from C. jeikeium or E. coli.

59. The microorganism according to claim 53,
wherein the amount and/or activity of one or more of the proteins chosen from the group consisting of formate-THF-Synthetase, gcvP, gcvT, gcvH, lpd, lplA, lipA or lipB are increased by increasing the copy number of one or more of nucleic acid sequences chosen from the group of sequences encoding formate-THF-Synthetase, gcvP, gcvT, gcvH, lpd, IplA, lipA or lipB, increasing transcription and/or translation of the nucleic acid sequences chosen from the group of sequences encoding formate-THF-Synthetase, gcvP, gcvT, gcvH, lpd, IplA, lipA or lipB or a combination thereof.

60. The microorganism according to claim 59,
wherein the gene copy number is increased by using autonomously replicating vectors comprising nucleic acid sequences chosen from the group of sequences consisting of sequences encoding formate-THF-Synthetase, gcvP, gcvT, gcvH, lpd, IplA, lipA or lipB and/or by chromosomal integration of additional copies of said nucleic acid sequences encoding formate-THF-Synthetase, gcvP, gcvT, gcvH, lpd, IplA, lipA or lipB into the genome of the starting organism.

61. The microorganism according to claim 60,
wherein transcription is increased by using a strong promoter which is preferably selected from the group comprising P_{EFTu}, P_{groES}, P_{SOD}, P₁₅, and λP_{R}.

62. A method of producing methionine in a microorganism comprising the step of:
cultivating a microorganism wherein the microorganism is according to claim 46.

63. The method according to claim 62,
wherein the microorganism is cultivated in the presence of lipoic acid and/or lipoamide.
